SEQUENCE LISTING (131 sequences)

SEQ ID NO: 1 - DNA, 900 bp, Human
tgcccccgcgccccaagcataaaccctggcgcgctcgcggcccggcactcttctggtccccacagactcagagagaacccaccatggtgctgtctcctgccgacaagaccaacgtcaaggccgcctggggtaaggtcggcgcgcacgctggcgagtatggtgcggaggccctggagaggtgaggctccctcccctgctccgacccgggctcctcgcccgcccggacccacaggccaccctcaaccgtcctggccccggacccaaaccccacccctcactctgcttctccccgcaggatgttcctgtccttccccaccaccaagacctacttcccgcacttcgacctgagccacggctctgcccaggttaagggccacggcaagaaggtggccgacgcgctgaccaacgccgtggcgcacgtggacgacatgcccaacgcgctgtccgccctgagcgacctgcacgcgcacaagcttcgggtggacccggtcaacttcaaggtgagcggcgggccgggagcgatctgggtcgaggggcgagatggcgccttcctcgcagggcagaggatcacgcgggttgcgggaggtgtagcgcaggcggcggctgcggacctgggccctcggccccactgaccctcttctctgcacagctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtggccatgcttcttgccccttgggcctccccccagcccctcctccccttcctgcacccgtacccccgtggtctttgaataaagtctgagtgggcggcagcctgtgtgtg

SEQ ID NO: 2 - DNA, 4314 bp, Human
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac

SEQ ID NO: 3 - DNA, 3007 bp, Human
ggagtttctggagggctgaacacgtggaggcaaacaggaaggtgaagaagaacttatcctatcaggacggaaggtcctgtgctcgggatcttccagacgtcgcgactctaaattgccccctctgaggtcaaggaacacaagatggttttggaaatgctgaacccgatacattataacatcaccagcatcgtgcctgaagccatgcctgctgccaccatgccagtcctgctcctcactggcctttttctcttggtgtggaattatgagggcacatcctcaataccaggtcctggctactgcatgggaattggacccctcatctcccacggcagattcctgtggatggggatcggcagtgcctgcaactactacaaccgggtatatggagaattcatgcgagtctggatctctggagaggaaacactcattatcagcaagtcctcaagtatgttccacataatgaagcacaatcattacagctctcgattcggcagcaaacttgggctgcagtgcatcggtatgcatgagaaaggcatcatatttaacaacaatccagagctctggaaaacaactcgacccttctttatgaaagctctgtcaggccccggccttgttcgtatggtcacagtctgtgctgaatccctcaaaacacatctggacaggttggaggaggtgaccaatgaatcgggctatgtggacgtgttgacccttctgcgtcgtgtcatgctggacacctctaacacgctcttcttgaggatccctttggacgaaagtgctatcgtggttaaaatccaaggttattttgatgcatggcaagctctcctcatcaaaccagacatcttctttaagatttcttggctatacaaaaagtatgagaagtctgtcaaggatttgaaagatgccatagaagttctgatagcagaaaaaagacgcaggatttccacagaagagaaactggaagaatgtatggactttgccactgagttgattttagcagagaaacgtggtgacctgacaagagagaatgtgaaccagtgcatattggaaatgctgatcgcagctcctgacaccatgtctgtctctttgttcttcatgctatttctcattgcaaagcaccctaatgttgaagaggcaataataaaggaaatccagactgttattggtgagagagacataaagattgatgatatacaaaaattaaaagtgatggaaaacttcatttatgagagcatgcggtaccagcctgtcgtggacttggtcatgcgcaaagccttagaagatgatgtaatcgatggctacccagtgaaaaaggggacaaacattatcctgaatattggaaggatgcacagactcgagtttttccccaaacccaatgaatttactcttgaaaattttgcaaagaatgttccttataggtactttcagccatttggctttgggccccgtggctgtgcaggaaagtacatcgccatggtgatgatgaaagccatcctcgttacacttctgagacgattccacgtgaagacattgcaaggacagtgtgttgagagcatacagaagatacacgacttgtccttgcacccagatgagactaaaaacatgctggaaatgatctttaccccaagaaactcagacaggtgtctggaacactagagaaggctggtcagtacctactctggagcatttctcatcagtagttcacatacaaatcatccatccttgccaatagtgtcatcctcacagtgaacactcagtggcccatggcattttataggcatacctcctatgggttgtcaccaagctaggtgctattggtcatctgctcctgttcacaccagagaaccaggctacaagagaaaaagcagaggccaagagtttgagggagaaatagtcggtgaagaaaccgtatccataaagacccgattccaccaaatgtgctttgagaaggataggccttcattaacaaaatgtatgtctggttccccagtagagctctactgcctcaacccaaggggatttttatgtctggggcagaaacactcaagttgattagaaagaccaggccaatgtcagggtacctggggccaaacccacctgctagtgtgaattaaagtactttaattttgttttctgtggaggtggaaaagcaacattcatagtctttggagaaatgcttagaaattcagcatttgacccttgctgtgaattaagcccaattaattcctgtttgtctacatatgatctgtctgtggcaaaagtttaatcagaggaaattctttcccagtctgtcgatttatgcctcagccacttgcctgtgctacaattcattgtgttacctgtagattcaggtaatacaaactatatataatcatcaagtaatacaaactaatttagtaatagcctgggttaagtattattagggccctgtgtctgctgtagaaaaaaaaattcacatgatgcacttcaaattcaaataaaaatccttttggcatgttcccatttttgcttagctcaattagtgtggctaaccaagagataactgtaaatgtgacattgatttgctcttactacagcttcagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctgaagtattttttaatcctacctcaaaatttaacagttacctgagagatttaacattatctagttcattgaatcattgtatgtggtcatggataaattgcacaccttggaattcgctttctaaaggaaatcaaatgaatggaggaactttccaaacaccactttacttgtgttatatagccaatataactatctctactgaatgtcattgaaaaactaaaaaattaaacttatttacaaataggtaaaaaaaaaaaaaaaaaaaaa

SEQ ID NO: 4 - DNA, 6030 bp, Human
gttggcccccgttacttttcctctgggaaatatggcgcacgctgggagaacagggtacgataaccgggagatagtgatgaagtacatccattataagctgtcgcagaggggctacgagtgggatgcgggagatgtgggcgccgcgcccccgggggccgcccccgcgccgggcatcttctcctcgcagcccgggcacacgccccatacagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccaggcagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtgaagtcaacatgcctgccccaaacaaatatgcaaaaggttcactaaagcagtagaaataatatgcattgtcagtgatgttccatgaaacaaagctgcaggctgtttaagaaaaaataacacacatataaacatcacacacacagacagacacacacacacacaacaattaacagtcttcaggcaaaacgtcgaatcagctatttactgccaaagggaaatatcatttattttttacattattaagaaaaaaagatttatttatttaagacagtcccatcaaaactcctgtctttggaaatccgaccactaattgccaagcaccgcttcgtgtggctccacctggatgttctgtgcctgtaaacatagattcgctttccatgttgttggccggatcaccatctgaagagcagacggatggaaaaaggacctgatcattggggaagctggctttctggctgctggaggctggggagaaggtgttcattcacttgcatttctttgccctgggggctgtgatattaacagagggagggttcctgtggggggaagtccatgcctccctggcctgaagaagagactctttgcatatgactcacatgatgcatacctggtgggaggaaaagagttgggaacttcagatggacctagtacccactgagatttccacgccgaaggacagcgatgggaaaaatgcccttaaatcataggaaagtatttttttaagctaccaattgtgccgagaaaagcattttagcaatttatacaatatcatccagtaccttaagccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctggtcctggaactgagccggggccctcactggcctcctccagggatgatcaacagggcagtgtggtctccgaatgtctggaagctgatggagctcagaattccactgtcaagaaagagcagtagaggggtgtggctgggcctgtcaccctggggccctccaggtaggcccgttttcacgtggagcatgggagccacgacccttcttaagacatgtatcactgtagagggaaggaacagaggccctgggcccttcctatcagaaggacatggtgaaggctgggaacgtgaggagaggcaatggccacggcccattttggctgtagcacatggcacgttggctgtgtggccttggcccacctgtgagtttaaagcaaggctttaaatgactttggagagggtcacaaatcctaaaagaagcattgaagtgaggtgtcatggattaattgacccctgtctatggaattacatgtaaaacattatcttgtcactgtagtttggttttatttgaaaacctgacaaaaaaaaagttccaggtgtggaatatgggggttatctgtacatcctggggcattaaaaaaaaaatcaatggtggggaactataaagaagtaacaaaagaagtgacatcttcagcaaataaactaggaaatttttttttcttccagtttagaatcagccttgaaacattgatggaataactctgtggcattattgcattatataccatttatctgtattaactttggaatgtactctgttcaatgtttaatgctgtggttgatatttcgaaagctgctttaaaaaaatacatgcatctcagcgtttttttgtttttaattgtatttagttatggcctatacactatttgtgagcaaaggtgatcgttttctgtttgagatttttatctcttgattcttcaaaagcattctgagaaggtgagataagccctgagtctcagctacctaagaaaaacctggatgtcactggccactgaggagctttgtttcaaccaagtcatgtgcatttccacgtcaacagaattgtttattgtgacagttatatctgttgtccctttgaccttgtttcttgaaggtttcctcgtccctgggcaattccgcatttaattcatggtattcaggattacatgcatgtttggttaaacccatgagattcattcagttaaaaatccagatggcaaatgaccagcagattcaaatctatggtggtttgacctttagagagttgctttacgtggcctgtttcaacacagacccacccagagccctcctgccctccttccgcgggggctttctcatggctgtccttcagggtcttcctgaaatgcagtggtgcttacgctccaccaagaaagcaggaaacctgtggtatgaagccagacctccccggcgggcctcagggaacagaatgatcagacctttgaatgattctaatttttaagcaaaatattattttatgaaaggtttacattgtcaaagtgatgaatatggaatatccaatcctgtgctgctatcctgccaaaatcattttaatggagtcagtttgcagtatgctccacgtggtaagatcctccaagctgctttagaagtaacaatgaagaacgtggacgcttttaatataaagcctgttttgtcttctgttgttgttcaaacgggattcacagagtatttgaaaaatgtatatatattaagaggtcacgggggctaattgctggctggctgccttttgctgtggggttttgttacctggttttaataacagtaaatgtgcccagcctcttggccccagaactgtacagtattgtggctgcacttgctctaagagtagttgatgttgcattttccttattgttaaaaacatgttagaagcaatgaatgtatataaaagcctcaactagtcatttttttctcctcttcttttttttcattatatctaattattttgcagttgggcaacagagaaccatccctattttgtattgaagagggattcacatctgcatcttaactgctctttatgaatgaaaaaacagtcctctgtatgtactcctctttacactggccagggtcagagttaaatagagtatatgcactttccaaattggggacaagggctctaaaaaaagccccaaaaggagaagaacatctgagaacctcctcggccctcccagtccctcgctgcacaaatactccgcaagagaggccagaatgacagctgacagggtctatggccatcgggtcgtctccgaagatttggcaggggcagaaaactctggcaggcttaagatttggaataaagtcacagaatcaaggaagcacctcaatttagttcaaacaagacgccaacattctctccacagctcacttacctctctgtgttcagatgtggccttccatttatatgtgatctttgttttattagtaaatgcttatcatctaaagatgtagctctggcccagtgggaaaaattaggaagtgattataaatcgagaggagttataataatcaagattaaatgtaaataatcagggcaatcccaacacatgtctagctttcacctccaggatctattgagtgaacagaattgcaaatagtctctatttgtaattgaacttatcctaaaacaaatagtttataaatgtgaacttaaactctaattaattccaactgtacttttaaggcagtggctgtttttagactttcttatcacttatagttagtaatgtacacctactctatcagagaaaaacaggaaaggctcgaaatacaagccattctaaggaaattagggagtcagttgaaattctattctgatcttattctgtggtgtcttttgcagcccagacaaatgtggttacacactttttaagaaatacaattctacattgtcaagcttatgaaggttccaatcagatctttattgttattcaatttggatctttcagggattttttttttaaattattatgggacaaaggacatttgttggaggggtgggagggaggaacaatttttaaatataaaacattcccaagtttggatcagggagttggaagttttcagaataaccagaactaagggtatgaaggacctgtattggggtcgatgtgatgcctctgcgaagaaccttgtgtgacaaatgagaaacattttgaagtttgtggtacgacctttagattccagagacatcagcatggctcaaagtgcagctccgtttggcagtgcaatggtataaatttcaagctggatatgtctaatgggtatttaaacaataaatgtgcagttttaactaacaggatatttaatgacaaccttctggttggtagggacatctgtttctaaatgtttattatgtacaatacagaaaaaaattttataaaattaagcaatgtgaaactgaattggagagtgataatacaagtcctttagtcttacccagtgaatcattctgttccatgtctttggacaaccatgaccttggacaatcatgaaatatgcatctcactggatgcaaagaaaatcagatggagcatgaatggtactgtaccggttcatctggactgccccagaaaaataacttcaagcaaacatcctatcaacaacaaggttgttctgcataccaagctgagcacagaagatgggaacactggtggaggatggaaaggctcgctcaatcaagaaaattctgagactattaataaataagactgtagtgtagatactgagtaaatccatgcacctaaaccttttggaaaatctgccgtgggccctccagatagctcatttcattaagtttttccctccaaggtagaatttgcaagagtgacagtggattgcatttcttttggggaagctttcttttggtggttttgtttattataccttcttaagttttcaaccaaggtttgcttttgttttgagttactggggttatttttgttttaaataaaaataagtgtacaataagtgtttttgtattgaaagcttttgttatcaagattttcatacttttaccttccatggctctttttaagattgatacttttaagaggtggctgatattctgcaacactgtacacataaaaaatacggtaaggatactttacatggttaaggtaaagtaagtctccagttggccaccattagctataatggcactttgtttgtgttgttggaaaaagtcacattgccattaaactttccttgtctgtctagttaatattgtgaagaaaaataaagtacagtgtgagatactg

SEQ ID NO: 5 - DNA, 2974 bp, Human
ctcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaaaaa

SEQ ID NO: 6 - DNA, 1977 bp, Human
cactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagaacctcatctcaagccaggcctcgagctacttcattgacgctgccacagtcgacgacagtggagagtacaggtgccagacaaacctctccaccctcagtgacccggtgcagctagaagtccatgtcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggagtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagtaaaacaacaatacaggctagggatggtaatcctttaaacatacaaaaattgctcgtattataaattacccagtttagaggggaaaaaagaaaataattattcctaaacaaatggataagtagaattaatgattgaggcaggaccctacagagtgtgggaactgctggggatctagagaattcagtgggaccaatgaaagcatggctgagaaatagcagggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcagtcagttccaatgaggtggggatggagaagacaattgttgcttatgaaaaaaagctttagctgtctcctgttttgtaagctttcagtgcaacatttcttggttccaataaagcattttac

SEQ ID NO: 7 - DNA, 3189 bp, Human
tttccagccatggctgccattacctgaccagcgccacagccggtctctctgcaggcgccgggagaagtgaccagagcaatttctgcttttcacagggcgggtttctcaacggtgacttgtgggcagtgccttctgctgagcgagtcatggcccgaaggcagaactaactgtgcctgcagtcttcactctcaggatgcagccgaggtgggcccaaggggccacgatgtggcttggagtcctgctgacccttctgctctgttcaagccttgagggtcaagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccaggtgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccacgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctaacaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcggaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaacttagacagcaaggccagatgcacatccctggaaggacatccatgttccgagaagaacagatgatccctgtatttcaagacctctgtgcacttatttatgaacctgccctgctcccacagaacacagcaattcctcaggctaagctgccggttcttaaatccatcctgctaagttaatgttgggtagaaagagatacagaggggctgttgaatttcccacatacactccttccaccaagttggaacatccttggaaattggaagagcacaagaggagatccagggcaaggccattgggatattctgaaacttgaatattttgttttgtgcagagataaagaccttttccatgcaccctcatacacagaaaccaattttcttttttatactcaatcatttctagcgcatggcctggttagaggctggttttttctcttttcctttggtccttcaaaggcttgtagttttgggtagtccttgttctttggaaatacacagtgctgaccagacagcctccccctgtcccctctatgacctcgccctccacaaatgggaaaaccagactacttgggagcaccgcctgtgaaataccaacctgaagacacggttcattcaggcaacgcacaaaacagaaaatgaaggtggaacaagcacagatgttcttcaactgtttttgtctacactctttctcttttcctctaccatgctgaaggctgaaagacaggaagatggtgccatcagcaaatattattcttaattgaaaacttgaaaaaaaaaaaaaaaa

SEQ ID NO: 8 - DNA, 1494 bp, Human
agtgaaacagaaggggaggtgcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtaaa

SEQ ID NO: 9 - DNA, 5026 bp, Human
agaggaggaaattgttcctcgtctgataagacaacagtggagaaaggacgcatgctgtttcttagggacacggctgacttccagatatgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatacttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttc

SEQ ID NO: 10 - DNA, 2301 bp, Human
tcgacagctctctcgcccagcccagttctggaagggataaaaagggggcatcaccgttcctgggtaacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagaggaggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatcacagagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatat

SEQ ID NO: 11 - DNA, 1752 bp, Human
ctgctccttctaggatctccgcctggttcggcccgcctgcctccactcctgcctccaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttttggtccaaaataaaacctcagctagctctgccaaaccc

SEQ ID NO: 12 - DNA, 2145 bp, Human
cagcatcaccatgtctgttcgatacagctcaagcaagcactactcttcctcccgcagtggaggaggaggaggaggaggaggatgtggaggaggaggaggagtgtcatccctaagaatttctagcagcaaaggctcccttggtggaggatttagctcaggggggttcagtggtggctcttttagccgtgggagctctggtgggggctgctttgggggctcatcaggtggctatggaggattaggaggttttggtggaggtagctttcgtggaagctatggaagtagcagctttggtgggagttatggaggcagctttggagggggcagtttcggaggtggcagctttggtgggggcagctttggtggaggcggctttggtggaggcggctttggaggaggctttggtggtggatttggaggagatggtggccttctctctggaaatgaaaaagtaaccatgcagaatctgaatgaccgcctggcttcctacttggacaaagttcgggctctggaagaatcaaactatgagctggaaggcaaaatcaaggagtggtatgaaaagcatggcaactcacatcagggggagcctcgtgactacagcaaatactacaaaaccatcgatgaccttaaaaatcagattctcaacctaacaactgataatgccaacatcctgcttcagatcgacaatgccaggctggcagctgatgacttcaggctgaagtatgagaatgaggtagctctgcgccagagcgtggaggctgacatcaacggcctgcgtagggtgctggatgagctgaccctgaccaaggctgacctggagatgcaaattgagagcctgactgaagagctggcctatctgaagaagaaccacgaggaggaaatgaaagaccttcgaaatgtgtccactggtgatgtgaatgtggaaatgaatgctgccccgggtgttgatctgactcaacttctgaataacatgagaagccaatatgaacaacttgctgaacaaaaccgcaaagatgctgaagcctggttcaatgaaaagagcaaggaactgactacagaaattgataataacattgaacagatatccagctataaatctgagattactgaattgagacgtaatgtacaagctctggagatagaactacagtcccaactggccttgaaacaatccctggaagcctccttggcagaaacagaaggtcgctactgtgtgcagctctcacagattcaggcccagatatccgctctggaagaacagttgcaacagattcgagctgaaaccgagtgccagaatactgaataccaacaactcctggatattaagatccgactggagaatgaaattcaaacctaccgcagcctgctagaaggagagggaagttccggaggcggcggacgcggcggcggaagtttcggcggcggctacggcggcggaagctccggcggcggaagctccggcggcggctacggcggcggccacggcggcagttccggcggcggctacggaggcggaagctccggcggcggaagctccggcggcggctacgggggcggaagctccagcggcggccacggcggcagttccagcggcggctacggtggtggcagttccggcggcggcggcggcggctacgggggcggcagctccggcggcggcagcagctccggcggcggatacggcggcggcagctccagcggaggccacaagtcctcctcttccgggtccgtgggcgagtcttcatctaagggaccaagatactaacaaaaccagagtaatcaagacaattattgaagaggtggcgcccgacggtagagttctttcatctatggttgaatcagaaaccaagaaacactactattaaactgcatcaagaggaaagagtctcccttcacacagaccattatttacagatgcatggaaaacaaagtctccaagaaaacacttctgtcttgatggtctatggaaatagaccttgaaaataaggtgtctacaaggtgttttgtggtttctgtatttcttcttttcactttaccagaaagtgttctttaatggaaagaaaaacaactttctgttctcatttactaatgaatttcaataaactttcttactgatgcaaactaaaaaaaaaaaaaaaaaaaaaa

SEQ ID NO: 13 - DNA, 1485 bp, Human
tccggggcgggggcggggcctcactctgcgatataactcgggtcgcgcggctcgcgcaggccgccaccgtcgtccgcaaagcctgagtcctgtcctttctctctccccggacagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaagccagcagaagcagggtaccctttggggagcaggaggccaataaaaagttcagagttcaaaaaaaaaaaaaaaaaaa

SEQ ID NO: 14 - DNA, 1513 bp, Human
cgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaaaaaaaaaaaaaaaaaaaaaaaaaaaaaaaa

SEQ ID NO: 15 - DNA, 1817 bp, Human
caaccatcctgaagctacaggtgctccctcctggaatctccaatggatttcagtcgcagaagcttccacagaagcctgagctcctccttgcaggcccctgtagtcagtacagtgggcatgcagcgcctcgggacgacacccagcgtttatgggggtgctggaggccggggcatccgcatctccaactccagacacacggtgaactatgggagcgatctcacaggcggcggggacctgtttgttggcaatgagaaaatggccatgcagaacctaaatgaccgtctagcgagctacctagaaaaggtgcggaccctggagcagtccaactccaaacttgaagtgcaaatcaagcagtggtacgaaaccaacgccccgagggctggtcgcgactacagtgcatattacagacaaattgaagagctgcgaagtcagattaaggatgctcaactgcaaaatgctcggtgtgtcctgcaaattgataatgctaaactggctgctgaggacttcagactgaagtatgagactgagagaggaatacgtctaacagtggaagctgatctccaaggcctgaataaggtctttgatgacctaaccctacataaaacagatttggagattcaaattgaagaactgaataaagacctagctctcctcaaaaaggagcatcaggaggaagtcgatggcctacacaagcatctgggcaacactgtcaatgtggaggttgatgctgctccaggcctgaaccttggcgtcatcatgaatgaaatgaggcagaagtatgaagtcatggcccagaagaaccttcaagaggccaaagaacagtttgagagacagactgcagttctgcagcaacaggtcacagtgaatactgaagaattaaaaggaactgaggttcaactaacggagctgagacgcacctcccagagccttgagatagaactccagtcccatctcagcatgaaagagtctttggagcacactctagaggagaccaaggcccgttacagcagccagttagccaacctccagtcgctgttgagctctctggaggcccaactgatgcagattcggagtaacatggaacgccagaacaacgaataccatatccttcttgacataaagactcgacttgaacaggaaattgctacttaccgccgccttctggaaggagaagacgtaaaaactacagaatatcagttaagcaccctggaagagagagatataaagaaaaccaggaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatcaatttgtaggtaatgttcctgaaaattgcaatacatttcaattatactaaacctcacaaagtagaggaatccatgtaaattgcaaataaaccactttctaattttttcctgtttctgaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 5435 bp, Human
  misc_feature (9)..(9): n is a, c, g, or t
cccgggagntggagcttgcagtgagccaagatggtgccactgmmctccagcctgggtgacaaagcgagactccatctcaaaaaaagaaaaaaaagaaagcattgggaaagcctaaatggtctacaggccacttggaagagaagtgacacaactctcagaaagggaaagaatgaatgaatgaaagaatgtgctttctggggaggaacagtcagcatttttgataatttctgagaggcaggtcagtggagtagttaagaccatgaacgctagggccagattctgcttcttcatatttttacagccttgggaaagttattctctctgtgccacactttgtttatctgaaacatgaagacamctacagcatcttctttacagagtctttctaaaaattaaatgagttaatttttgtaaaatccttcaaagagtcccagccatatgttaagttccatgtaaatactttgctaataaaaataaaattttggagttataagcgcagagtttcttggataatactgttctatgtcctaaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagttacatggtagaacgaagaatagaccagaagccagaagtgctggcctttaaccatatacctgctattcgctagctgtgtaccctctgcaaatgtcctgggtcttagattggtgtctatgaattagacaatctttaagatctcttctagcctcccccatgttacgtgtcattttccttaggtttcttttaaagtgcccaaaacagtccacatagtaggtattgtggtgattaaacagaaaggcactgaaccacaaagcagacacttgattaacatcagttgtatctgactatgcctttttttgtttgtttgtttttttggaaacagagttccactctgtcgcccaggttcgagtgcaatggcmmaattacagctcactgcaacctccgcctcccatgttccagcaattctcctgcctcagcctcccaagtagctgggattacaggcatgcaccagcacgcccagctaatttttgtatttttagtagagatggggtttcaccatattggccaggctggtcttgaactcctgacctcagntgacccacctgcctcagcctcccaaagtgctgggattacaggtgtgagccactgctccccaagaagtcatatgacctgtgggrtctctagaggggacctcaagagaatggtgtgtgtcaggcactgcactagtgactttatgtagattgcttcatttgttcctgccaacagcccactgaaataagtattaaaattctgtgcccagtgaagaaacagagagggtgtttgtgatgctcggaaaccaataatattgtgtgtagctctcaactcaagttcagggwwwgctgacaccttgccatttgtctcctcaaatggtcgtatgtatttccaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagaggtaagtgctttataaagcactcaatttctactcagaaatttttgatggccttaagttcctctactcgtttctatccttcctactcactgtcctcccggaatccactaccgattttctatttcttgcctcgtattgtctgactggctcacttggatttatctcacggagtctggattttctacccgggctcacctccgtccctccatatttgtcctccactttcacagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 17 - DNA, 3582 bp, unknown
  no Organism listed
acagaagaaatagcaagtgccgagaagctggcatcagaaaaacagaggggagatttgtgtggctgcagccgagggagaccaggaagatctgcatggtgggaaggacctgatgatacagaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagcacagcctctccccatccctccagccttatctgtcatcaccatcaacccctcccataccacctaaacaaaatctaacttgtaattccttgaacatgtcaggacatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgtgagtgtacatgccaaagtgtgcctctctctcttgacccattatttcagacttaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaattgcctagacccttatttttattaatttccaatagatgctgcctatgggctaatattgctttagatgaacattagatatttaaagtctaagaggttcaaaatccaactcattatcttctctttctttcacctcccctgctcctctccctatattactgattgactgaacaggatggtccccaagatgccagtcaaatgagaaacccagtggctccttgtggatcatgcatgcaagactgctgaagccagaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttt

SEQ ID NO: 18 - DNA, 4407 bp, Human
tttcgactcgcgctccggctgctgtcacttggctctctggctggagcttgaggacgcaaggagggtttgtcactggcagactcgagactgtaggcactgccatggcccctgtgctcagtaaggactcggcggacatcgagagtatcctggctttaaatcctcgaacacaaactcatgcaactctgtgttccacttcggccaagaaattagacaagaaacattggaaaagaaatcctgataagaactgctttaattgtgagaagctggagaataattttgatgacatcaagcacacgactcttggtgagcgaggagctctccgagaagcaatgagatgcctgaaatgtgcagatgccccgtgtcagaagagctgtccaactaatcttgatattaaatcattcatcacaagtattgcaaacaagaactattatggagctgctaagatgatattttctgacaacccacttggtctgacttgtggaatggtatgtccaacctctgatctatgtgtaggtggatgcaatttatatgccactgaagagggacccattaatattggtggattgcagcaatttgctactgaggtattcaaagcaatgagtatcccacagatcagaaatccttcgctgcctcccccagaaaaaatgtctgaagcctattctgcaaagattgctctttttggtgctgggcctgcaagtataagttgtgcttcctttttggctcgattggggtactctgacatcactatatttgaaaaacaagaatatgttggtggtttaagtacttctgaaattcctcagttccggctgccgtatgatgtagtgaattttgagattgagctaatgaaggaccttggtgtaaagataatttgcggtaaaagcctttcagtgaatgaaatgactcttagcactttgaaagaaaaaggctacaaagctgctttcattggaataggtttgccagaacccaataaagatgccatcttccaaggcctgacgcaggaccaggggttttatacatccaaagactttttgccacttgtagccaaaggcagtaaagcaggaatgtgcgcctgtcactctccattgccatcgatacggggagtcgtgattgtacttggagctggagacactgccttcgactgtgcaacatctgctctacgttgtggagctcgccgagtgttcatcgtcttcagaaaaggctttgttaatataagagctgtccctgaggagatggagcttgctaaggaagaaaagtgtgaatttctgccattcctgtccccacggaaggttatagtaaaaggtgggagaattgttgctatgcagtttgttcggacagagcaagatgaaactggaaaatggaatgaagatgaagatcagatggtccatctgaaagccgatgtggtcatcagtgcctttggttcagttctgagtgatcctaaagtaaaagaagccttgagccctataaaatttaacagatggggtctcccagaagtagatccagaaactatgcaaactagtgaagcatgggtatttgcaggtggtgatgtcgttggtttggctaacactacagtggaatcggtgaatgatggaaagcaagcttcttggtacattcacaaatacgtacagtcacaatatggagcttccgtttctgccaagcctgaactacccctcttttacactcctattgatctggtggacattagtgtagaaatggccggattgaagtttataaatccttttggtcttgctagcgcaactccagccaccagcacatcaatgattcgaagagcttttgaagctggatggggttttgccctcaccaaaactttctctcttgataaggacattgtgacaaatgtttcccccagaatcatccggggaaccacctctggccccatgtatggccctggacaaagctcctttctgaatattgagctcatcagtgagaaaacggctgcatattggtgtcaaagtgtcactgaactaaaggctgacttcccagacaacattgtgattgctagcattatgtgcagttacaataaaaatgactggacggaacttgccaagaagtctgaggattctggagcagatgccctggagttaaatttatcatgtccacatggcatgggagaaagaggaatgggcctggcctgtgggcaggatccagagctggtgcggaacatctgccgctgggttaggcaagctgttcagattcctttttttgccaagctgaccccaaatgtcactgatattgtgagcatcgcaagagctgcaaaggaaggtggtgccaatggcgttacagccaccaacactgtctcaggtctgatgggattaaaatctgatggcacaccttggccagcagtggggattgcaaagcgaactacatatggaggagtgtctgggacagcaatcagacctattgctttgagagctgtgacctccattgctcgtgctctgcctggatttcccattttggctactggtggaattgactctgctgaaagtggtcttcagtttctccatagtggtgcttccgtcctccaggtatgcagtgccattcagaatcaggatttcactgtgatcgaagactactgcactggcctcaaagccctgctttatctgaaaagcattgaagaactacaagactgggatggacagagtccagctactgtgagtcaccagaaagggaaaccagttccacgtatagctgaactcatggacaagaaactgccaagttttggaccttatctggaacagcgcaagaaaatcatagcagaaaacaagattagactgaaagaacaaaatgtagctttttcaccacttaagagaagctgttttatccccaaaaggcctattcctaccatcaaggatgtaataggaaaagcactgcagtaccttggaacatttggtgaattgagcaacgtagagcaagttgtggctatgattgatgaagaaatgtgtatcaactgtggtaaatgctacatgacctgtaatgattctggctaccaggctatacagtttgatccagaaacccacctgcccaccataaccgacacttgtacaggctgtactctgtgtctcagtgtttgccctattgtcgactgcatcaaaatggtttccaggacaacaccttatgaaccaaagagaggcgtacccttatctgtgaatccggtgtgttaaggtgatttgtgaaacagttgctgtgaactttcatgtcacctacatatgctgatctcttaaaatcatgatccttgtgttcagctctttccaaattaaaacaaatatacattttctaaataaaaatatgtaatttcaaaatacatttgtaagtgtaaaaaatgtctcatgtcaatgaccattcaattagtggcataaaatagaataattcttttctgaggatagtagttaaataactgtgtggcagttaattggatgttcactgccagttgtcttatgtgaaaaattaactttttgtgtggcaattagtgtgacagtttccaaattgccctatgctgtgctccatatttgatttctaattgtaagtgaaattaagcattttgaaacaaagtactctttaacatacaagaaaatgtatccaaggaaacattttatcaataaaaattacctttaattttaatgctgtttctaagaaaatgtagttagctccataaagtacaaatgaagaaagtcaaaaattatttgctatggcaggataagaaagcctaaaattgagtttgtggactttattaagtaaaatccccttcgctgaaattgcttatttttggtgttggatagaggatagggagaatatttactaactaaataccattcactactcatgcgtgagatgggtgtacaaactcatcctcttttaatggcatttctctttaaactatgttcctaaccaaatgagatgataggatagatcctggttaccactcttttactgtgcacatatgggccccggaattctttaatagtcaccttcatgattatagcaactaatgtttgaacaaagctcaaagtatgcaatgcttcattattcaagaatgaaaaatataatgttgataatatatattaagtgtgccaaatcagtttgactactctctgttttagtgtttatgtttaaaagaaatatattttttgttattattagataatatttttgtatttctctattttcataatcagtaaatagtgtcatataaactcatttatctcctcttcatggcatcttcaatatgaatctataagtagtaaatcagaaagtaacaatctatggcttatttctatgacaaattcaagagctagaaaaataaaatgtttcattatgcacttttagaaatgcatatttgccacaaaacctgtattactgaataatatcaaataaaatatcataaagcatttt

SEQ ID NO: 19 - DNA, 5616 bp, Human
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 20 - DNA, 2805 bp, Human
cgggtctgatagtccctacctgtcaggactggtgttaggatgagataatgtttgtgaactgtaaacatatataaacgtgtgctactgtgagaactggaacaaagaagagagggagtgagagaaatcaagggagggctggggctgggaaagaacgaaaagggagtcgcgtatagaggagaggcgacagtcgcgagccacactttgcaatgaaactctttagactttctgccgggagagcggcccagacgcgccaggtctgtagcaggaggccgcgagggcgggtccccagaagcctacaggtgagtatcggttctccccttcccggctttcggtccggaggaggcgggagcagcttccctgttctgatcctatcgcgggcggcgcagggccggcttggccttccgtgggacggggaggggggcgggatgtgtcacccaaataccagtggggacggtcggtggtggaaccagccgggcaggtcgggtagagtataagagccggagggagcggccggggcgcagacgcctgcagaccatcccagacgccggagcccgagccccgacgagtccccgcgcctcatccgcccgcgtccggtccgcgttcctccgccccaccatggctcggggccccggcctcgcgccgccaccgctgcggctgccgctgctgctgctggtgctggcggcggtgaccggccacacggccgcgcaggacaactgcacgtgtcccaccaacaagatgaccgtgtgcagccccgacggccccggcggccgctgccagtgccgcgcgctgggctcgggcatggcggtcgactgctccacgctgacctccaagtgtctgctgctcaaggcgcgcatgagcgcccccaagaacgcccgcacgctggtgcggccgagtgagcacgcgctcgtggacaacgatggcctctacgaccccgactgcgaccccgagggccgcttcaaggcgcgccagtgcaaccagacgtcggtgtgctggtgcgtgaactcggtgggcgtgcgccgcacggacaagggcgacctgagcctacgctgcgatgagctggtgcgcacccaccacatcctcattgacctgcgccaccgccccaccgccggcgccttcaaccactcagacctggacgccgagctgaggcggctcttccgcgagcgctatcggctgcaccccaagttcgtggcggccgtgcactacgagcagcccaccatccagatcgagctgcggcagaacacgtctcagaaggccgccggtgaagtggatatcggcgatgccgcctactacttcgagagggacatcaagggcgagtctctattccagggccgcggcggcctggacttgcgcgtgcgcggagaacccctgcaggtggagcgcacgctcatctattacctggacgagattcccccgaagttctccatgaagcgcctcaccgccggcctcatcgccgtcatcgtggtggtcgtggtggccctcgtcgccggcatggccgtcctggtgatcaccaaccggagaaagtcggggaagtacaagaaggtggagatcaaggaactgggggagttgagaaaggaaccgagcttgtaggtacccggcggggcaggggatggggtggggtaccggatttcggtatcgtcccagacccaagtgagtcacgcttcctgattcctcggcgcaaaggagacgtttatcctttcaaattcctgccttccccctcccttttgcgcacacaccaggtttaatagatcctggcctcagggtctcctttctttctcacttctgtcttgagggaagcatttctaaaatgtatcccctttcggtccaacaacaggaaacctgactggggcagtgaaggaagggatggcacagcgttatgtgtaaaaaacaagtatctgtatgacaacccgggatcgtttgcaagtaactgaatccattgcgacattgtgaaggcttaaatgagtttagatgggaaatagcgttgttatcgccttgggtttaaattatttgatgagttccacttgtatcatggcctacccgaggagaagaggagtttgttaactgggcctatgtagtagcctcatttaccatcgtttgtattactgaccacatatgcttgtcactgggaaagaagcctgtttcagctgcctgaacgcagtttggatgtctttgaggacagacattgcccggaaactcagtctatttattcttcagcttgcccttactgccactgatattggtaatgttcttttttgtaaaatgtttgtacatatgttgtctttgataatgttgctgtaattttttaaaataaaacacgaatttaataaaatatgggaaaggcacaaaccagaagtcggcatttgtgaaaagtccctccagatttctatcactttggtctctaatttcccaagacttgtattttttttttatttcaaattataacacttttttttcccccagaagtgggtgtttcatgttgctactctggtgtgtcccaagatatcctaactggccagtgtaaatgctattctttctaaataagattatttggaaacttccttcaaactgcaggagggcgagctctgagggcacgagaagctaaaactagctgcttttgatgaaaaagagtgccagtctttggtcatctctaaacaaggcttatcaccaatggagacagaaaactctagttcaagagctgtacctcctttgaatcccagccctactcgaaataagtggtactatttccatttagcctttgagcaaatcacttaactcaaaggcgttgtggctctaagattaaacgacttt

SEQ ID NO: 21 - DNA, 6450 bp, Human
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 22 - DNA, 2745 bp, Human
ctcggtctttaaaaggaagaaggggcttatcgttaagtcgcttgtgatcttttttcagtttctccagctgctggctttttggacacccactcccccgccaggaggcagttgcaagcgcggaggctgcgagaaataactgcctcttgaaacttgcagggcgaagagcaggcggcgagcgctgggccggggagggaccacccgagctgcgacgggctctggggctgcggggcagggctggcgcccggagcctgagctgcaggaggtgcgctcgctttcctcaacaggtggcggcggggcgcgcgccgggagaccccccctaatgcgggaaaagcacgtgtccgcattttagagaaggcaaggccggtgtgtttatctgcaagccattatacttgcccacgaatctttgagaacattataatgacctttgtgcctcttcttgcaaggtgttttctcagctgttatctcaagacatggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagctctctctcactctcttggagattgtttatgctgagggaagccagctgccatggtgtgaggcagactcctggaggagcccacatgtctgtaagtagaagcagatcttttgaggcctgtcaacagccacgggaatgagcttggaagcagatcccacctcctccctcacacaagtcgagccttcagatgagcctgcagcctttgtcgacaccttgactgcattctcatgagagaccttgagccagagatacttagctaagccatgcccatggactcctgacccacagaaactgtgataataagtttgttgtttcaagctgctaacttatggagtaatatgttacacaaaaatagctaatatatagctcaaaactggaagcaacccaaatatctattaactggtagataaacaaactactcatttccaaacttatttccaaaactggaacactacttggcaatcaaataattaactatgcattaagtgtaacaacctggatgaatctcaaaggcattatgttaagtgaaacaagtgagccacgtaagactacatactgtttgattccctctatatgatattctagaaaaggcaaaactatagtaataggaaacagtgagtgatcacctagggttgaagacaggtgaaaggggattgactgcaaagaggcaggaggaaacgtcttgggagatggagatgttccttatattgatggcggtggtggttacacaactgcacttttatcaaaacttacctaactgctacttaaaataggtgtattaatatttttactgtatgtaaattatacctcaataaatttgatttaaaaaaaaaaaaaaaaaaaaaaaaaaaaaaaaaaaaaaaaaaaaaaaa

SEQ ID NO: 23 - DNA, 1101 bp, Human
ccggaagtgctgcgagccctgggccacgctggccgtgctggcagtgggccgcctcgatccctctgcagtctttcccttgaggctccaagaccagcaggtgaggcctcgcggcgctgaaaccgtgaggcccggaccacaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagcccggaggctgtttgatgtcctgcacgagcccttcttgaaagtaccctgatgaccccagctgccaaggaaacccccagtgtaataataaatcgtcctcccaggccaggctc

SEQ ID NO: 24 - DNA, 2651 bp, Human
tctttttcttttttttttaagaaaaactagtgacattgcagagaaggacgcttcctctctatcttttggcgcattagtgaagggggtattctattttgttaaagcgcccaagggggcgcagggaccttggagagaagagtggggaggaaagaggaagggtgggtggggggcagagggcgagtcggcggcggcgagggcaagctctttcttgcggcacgatgccgtctctgctggtgctcactttctccccgtgcgtactactcggctgggcgttgctggccggcggcaccggtggcggtggcgttggcggcggcggcggtggcgcgggcataggcggcggacgccaggagagagaggcgctgccgccacagaagatcgaggtgctggtgttactgccccaggatgactcgtacttgttttcactcacccgggtgcggccggccatcgagtatgctctgcgcagcgtggagggcaacgggactgggaggcggcttctgccgccgggcactcgcttccaggtggcttacgaggattcagactgtgggaaccgtgcgctcttcagcttggtggaccgcgtggcggcggcgcggggcgccaagccagaccttatcctggggccagtgtgcgagtatgcagcagcgccagtggcccggcttgcatcgcactgggacctgcccatgctgtcggctggggcgctggccgctggcttccagcacaaggactctgagtactcgcacctcacgcgcgtggcgcccgcctacgccaagatgggcgagatgatgctcgccctgttccgccaccaccactggagccgcgctgcactggtctacagcgacgacaagctggagcggaactgctacttcaccctcgagggggtccacgaggtcttccaggaggagggtttgcacacgtccatctacagtttcgacgagaccaaagacttggatctggaagacatcgtgcgcaatatccaggccagtgagagagtggtgatcatgtgtgcgagcagtgacaccatccggagcatcatgctggtggcgcacaggcatggcatgaccagtggagactacgccttcttcaacattgagctcttcaacagctcttcctatggagatggctcatggaagagaggagacaaacacgactttgaagctaagcaagcatactcgtccctccagacagtcactctactgaggacagtgaaacctgagtttgagaagttttccatggaggtgaaaagttcagttgagaaacaagggctcaatatggaggattacgttaacatgtttgttgaaggattccacgatgccatcctcctctacgtcttggctctacatgaagtactcagagctggttacagcaaaaaggatggagggaaaattatacagcagacttggaacagaacatttgaaggtatcgccgggcaggtgtccatagatgccaacggagaccgatatggggatttctctgtgattgccatgactgatgtggaggcgggcacccaggaggttattggtgattattttggaaaagaaggtcgttttgaaatgcggccgaatgtcaaatatccttggggccctttaaaactgagaatagatgaaaaccgaattgtagagcatacaaacagctctccctgcaaatcatgtggcctagaagaatcggcagtgacaggaattgtcgtgggggctttactaggagctggcttgctaatggccttctactttttcaggaagaaatacagaataaccattgagaggcgaacccagcaagaagaaagtaaccttggaaaacatcgggaattacgggaagattccatcagatcccatttttcagtagcttaaaggaagccccccactttttttttttctgcctgagattctttaaggagatagacgggttgaaagacatcaatgaaacagaaggggcgttcttgaagaattcataattttaagcagttagtaatttcattttaaaatttctgtagaagctcaggaattatgattaatcaccatctgcctccaggcctttcatctcatgacaaacaaatataataatgatatcgtgtcactctgttaaatgttcatactgtttcaagcccatatgattagatttatgtttttaaaatctgttgtctccatatcttgatggcttttgggagcatttcacacaaggatataaaatgcggttttcttaaatgaaatgttttgtagctagaataaaatcatttttacaagtacagcattcttggaaagaatttaacacccaaaaaggggaaaatgtaatgaaaaatctcaaggttggaaatacagccttactctctctagagctggaggacaggtttgtggttgaggacttctctgtccgatgtctacattcaggttctgacttcatatcttgaaaaaggatttcctccctgtctttttcagtgtctcataaacgctactctggattgttgtaaatattagtgagatgggaggatttacagaagaaaagcaagtcaaaaatatttcctttttgatgtaaaaaaaaaaagccctatttcgcactaacattttattttacaagtattttaatcttatattttggtattagaaaaatttgtctattttttcattttgaagattaaatgttgcttacattttaaaaaaaaa

SEQ ID NO: 25 - DNA, 2363 bp, Human
tcgagcccgctttccagggaccctacctgagggcccacaggtgaggcagcctggcctagcaggccccacgccaccgcctctgcctccaggccgcccgctgctgcggggccaccatgctcctgcccaggcctggagactgacccgaccccggcactacctcgaggctccgcccccacctgctggaccccagggtaaggacaagggcccccagactcacagttccagccctgaggacaggggttccctcatccccccacccagcctaatgcccacctcctaatagaggggttcctggggacctgaagagggggcactatgacgtctccccaagcacctaggtgttctgtcctgctcttccttcagactcagccgttggaccccagtcctttcctccccagacccaggagttccagccctcaggcccctcctccctcatactagggagtcctggcccccaaattcctcctttcccaagacttatgatttcaggtcctcagctgtctcctccctcaaaccgggatcctcagtcccctgctccaccaggctcaggcatgggggtccccatccctgcaaatccaggcgtccccccgctgctggtcagacactgaccccatccttgaacccagcccaatctgcgtccgtgatcacggcgtgctctggccaaggcccagtccctacagcctgcctggatggacgcctgggactgggggcgccaggactgggctgggctgggctcccccaggccctgcctccccgtccatctcctcacaggtcccaccctggcccaggaggtcagccagggaatcattaacaagaggcagtgacatggcgcagaaggagggtggccggactgtgccatgctgctccagacccaaggtggcagctctcactgcggggaccctgctacttctgacagccatcggggcggcatcctgggccattgtggctgttctcctcaggagtgaccaggagccgctgtacccagtgcaggtcagctctgcggacgctcggctcatggtctttgacaagacggaagggacgtggcggctgctgtgctcctcgcgctccaacgccagggtagccggactcagctgcgaggagatgggcttcctcagggcactgacccactccgagctggacgtgcgaacggcgggcgccaatggcacgtcgggcttcttctgtgtggacgaggggaggctgccccacacccagaggctgctggaggtcatctccgtgtgtgattgccccagaggccgtttcttggccgccatctgccaagactgtggccgcaggaagctgcccgtggaccgcatcgtgggaggccgggacaccagcttgggccggtggccgtggcaagtcagccttcgctatgatggagcacacctctgtgggggatccctgctctccggggactgggtgctgacagccgcccactgcttcccggagcggaaccgggtcctgtcccgatggcgagtgtttgccggtgccgtggcccaggcctctccccacggtctgcagctgggggtgcaggctgtggtctaccacgggggctatcttccctttcgggaccccaacagcgaggagaacagcaacgatattgccctggtccacctctccagtcccctgcccctcacagaatacatccagcctgtgtgcctcccagctgccggccaggccctggtggatggcaagatctgtaccgtgacgggctggggcaacacgcagtactatggccaacaggccggggtactccaggaggctcgagtccccataatcagcaatgatgtctgcaatggcgctgacttctatggaaaccagatcaagcccaagatgttctgtgctggctaccccgagggtggcattgatgcctgccagggcgacagcggtggtccctttgtgtgtgaggacagcatctctcggacgccacgttggcggctgtgtggcattgtgagttggggcactggctgtgccctggcccagaagccaggcgtctacaccaaagtcagtgacttccgggagtggatcttccaggccataaagactcactccgaagccagcggcatggtgacccagctctgaccggtggcttctcgctgcgcagcctccagggcccgaggtgatcccggtggtgggatccacgctgggccgaggatgggacgtttttcttcttgggcccggtccacaggtccaaggacaccctccctccagggtcctctcttccacagtggcgggcccactcagccccgagaccacccaacctcaccctcctgacccccatgtaaatattgttctgctgtctgggactcctgtctaggtgcccctgatgatgggatgctctttaaataataaagatggttttgatt

SEQ ID NO: 26 - DNA, 2855 bp, Human
agccccaaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagcaaggatgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatgatttcctagcaggacttacagaaataaagagctatcatgctgtggtttattatggtttgttacattgatgggatacatactgaaatcagcaaacaaaacagatgtatagattagagtgtggagaaaacagaggaaaacttgcagttacgaagactggcaacttggctttactaagttttcagactggcaggaagtcaaacctattaggctgaggaccttgtggagtgtagctgatccagctgatagaggaactagccaggtgggggcctttccctttggatggggggcatatctgacagttattctctccaagtggagacttacggacagcatataattctccctgcaaggatgtatgataatatgtacaaagtaattccaactgaggaagctcacctgatccttagtgtccaaggtttttactgggggtctgtaggacgagtatggagtacttgaataattgacctgaagtcctcagacctgaggttccctagagttcaaacagatacagcatggtccagagtcccagatgtacaaaaacagggattcatcacaaatcccatctttagcatgaagggtctggcatggcccaaggccccaagtatatcaaggcacttgggcagaacatgccaaggaatcaaatgtcatctcccaggagttattcaagggtgagccctttacttgggatgtacaggctttgagcagtgcagggctgctgagtcaaccttttattgtacaggggatgagggaaagggagaggatgaggaagcccccctggggatttggtttggtcttgtgatcaggtggtctatggggctatccctacaaagaagaatccagaaataggggcacattgaggaatgatactgagcccaaagagcattcaatcattgttttatttgccttcttttcacaccattggtgagggagggattaccaccctggggttatgaagatggttgaacaccccacacatagcaccggagatatgagatcaacagtttcttagccatagagattcacagcccagagcaggaggacgctgcacaccatgcaggatgacatgggggatgcgctcgggattggtgtgaagaagcaaggactgttagaggcaggctttatagtaacaagacggtggggcaaactctgatttccgtgggggaatgtcatggtcttgctttactaagttttgagactggcaggtagtgaaactcattaggctgagaaccttgtggaatgcagctgacccagctgatagaggaagtagccaggtgggagcctttcccagtgggtgtgggacatatctggcaagattttgtggcactcctggttacagatactggggcagcaaataaaactgaatcttgttttcagaccttaaaaaaaaaaaaaaaaaaaaa

SEQ ID NO: 27 - DNA, 4530 bp, Human
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 28 - DNA, 1356 bp, Human
ttctcccgcaaccttcccttcgctccctcccgtcccccccagctcctagcctccgactccctccccccctcacgcccgccctctcgccttcgccgaaccaaagtggattaattacacgctttctgtttctctccgtgctgttctctcccgctgtgcgcctgcccgcctctcgctgtcctctctccccctcgccctctcttcggcccccccctttcacgttcactctgtctctcccactatctctgcccccctctatccttgatacaacagctgacctcatttcccgataccttttcccccccgaaaagtacaacatctggcccgccccagcccgaagacagcccgtcctccctggacaatcagacgaattctccccccccccccaaaaaaaaaagccatccccccgctctgccccgtcgcacattcggcccccgcgactcggccagagcggcgctggcagaggagtgtccggcaggagggccaacgcccgctgttcggtttgcgacacgcagcagggaggtgggcggcagcgtcgccggcttccagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtgagcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacataccctcccccc

SEQ ID NO: 29 - DNA, 2830 bp, Human
ggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgctccgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcacgttctggcatcacttggcctttctggatgctggacacctgaagcttggaactcacctggccgaagctcgagcctcctgagtcctactgacctgtgctttctggtgtggagtccagggctgctatgaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagcaaggatgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatgatttcctagcaggacttacagaaataaagagctatcatgctgtggtttattatggtttgttacattgatgggatacatactgaaatcagcaaacaaaacagatgtatagattagagtgtggagaaaacagaggaaaacttgcagttacgaagactggcaacttggctttactaagttttcagactggcaggaagtcaaacctattaggctgaggaccttgtggagtgtagctgatccagctgatagaggaactagccaggtgggggcctttccctttggatggggggcatatctgacagttattctctccaagtggagacttacggacagcatataattctccctgcaaggatgtatgataatatgtacaaagtaattccaactgaggaagctcacctgatccttagtgtccaaggtttttactgggggtctgtaggacgagtatggagtacttgaataattgacctgaagtcctcagacctgaggttccctagagttcaaacagatacagcatggtccagagtcccagatgtacaaaaacagggattcatcacaaatcccatctttagcatgaagggtctggcatggcccaaggccccaagtatatcaaggcacttgggcagaacatgccaaggaatcaaatgtcatctcccaggagttattcaagggtgagccctttacttgggatgtacaggctttgagcagtgcagggctgctgagtcaaccttttattgtacaggggatgagggaaagggagaggatgaggaagcccccctggggatttggtttggtcttgtgatcaggtggtctatggggctatccctacaaagaagaatccagaaataggggcacattgaggaatgatactgagcccaaagagcattcaatcattgttttatttgccttcttttcacaccattggtgagggagggattaccaccctggggttatgaagatggttgaacaccccacacatagcaccggagatatgagatcaacagtttcttagccatagagattcacagcccagagcaggaggacgctgcacaccatgcaggatgacatgggggatgcgctcgggattggtgtgaagaagcaaggactgttagaggcaggctttatagtaacaagacggtggggcaaactctgatttccgtgggggaatgtcatggtcttgctttactaagttttgagactggcaggtagtgaaactcattaggctgagaaccttgtggaatgcagctgacccagctgatagaggaagtagccaggtgggagcctttcccagtgggtgtgggacatatctggcaagattttgtggcactcctggttacagatactggggcagcaaataaaactgaatcttgttttcagaccttaaaaaaaaaaaaaaaaaaaaa

SEQ ID NO: 30 - DNA, 1202 bp, Human
gcggccgctgcacagccatgcccgggcaagaactcaggacgctgaatggctctcagatgctcctggtgttgctggtgctctcgtggctgccgcatgggggcgccctgtctctggccgaggcgagccgcgcaagtttcccgggaccctcagagttgcacaccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgacctctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggaggcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttaaaaaaaaaaaaaaaaa

SEQ ID NO: 31 - DNA, 502 bp, Human
acagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 32 - DNA, 517 bp, Human
cctccacagcaacttccttgatccctgccacgcacgactgaacacagacagcagccgcctcgccatgaagctgctgatggtcctcatgctggcggccctcctcctgcactgctatgcagattctggctgcaaactcctggaggacatggttgaaaagaccatcaattccgacatatctatacctgaatacaaagagcttcttcaagagttcatagacagtgatgccgctgcagaggctatggggaaattcaagcagtgtttcctcaaccagtcacatagaactctgaaaaactttggactgatgatgcatacagtgtacgacagcatttggtgtaatatgaagagtaattaactttacccaaggcgtttggctcagagggctacagactatggccagaactcatctgttgattgctagaaaccacttttctttcttgtgttgtctttttatgtggaaactgctagacaactgttgaaacctcaaattcatttccatttcaataactaactgcaaatc

SEQ ID NO: 33 - DNA, 3069 bp, Human
tgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcggagcgcagccctgcgccgcggaccaggctccaaccaggcggcgaggcggccacacgcaccgagccagcgacccccgggcgacgcgcggggccagggagcgctacgatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatggcaacgccgatggggagtactgcaagttccccttcttgttcaatggcaaggagtacaacagctgcactgatactggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgacgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacactgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggctttcccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatcctttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggc

SEQ ID NO: 34 - DNA, 2334 bp, Human
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 35 - DNA, 5011 bp, Human
ccaggcggcgttgcggccccggccccggctccctgcgccgccgccgccgccgccgccgccgccgccgccgccgccgccagcgctagcgccagcagccgggcccgatcacccgccgcccggtgcccgccgccgcccgcgccagcaaccgggcccgatcacccgccgcccggtgcccgccgccgcccgcgccaccggcatggcgctccggggcttctgcagcgccgatggctccgacccgctctgggactggaatgtcacgtggaataccagcaaccccgacttcaccaagtgctttcagaacacggtcctcgtgtgggtgccttgtttttacctctgggcctgtttccccttctacttcctctatctctcccgacatgaccgaggctacattcagatgacacctctcaacaaaaccaaaactgccttgggatttttgctgtggatcgtctgctgggcagacctcttctactctttctgggaaagaagtcggggcatattcctggccccagtgtttctggtcagcccaactctcttgggcatcaccacgctgcttgctacctttttaattcagctggagaggaggaagggagttcagtcttcagggatcatgctcactttctggctggtagccctagtgtgtgccctagccatcctgagatccaaaattatgacagccttaaaagaggatgcccaggtggacctgtttcgtgacatcactttctacgtctacttttccctcttactcattcagctcgtcttgtcctgtttctcagatcgctcacccctgttctcggaaaccatccacgaccctaatccctgcccagagtccagcgcttccttcctgtcgaggatcaccttctggtggatcacagggttgattgtccggggctaccgccagcccctggagggcagtgacctctggtccttaaacaaggaggacacgtcggaacaagtcgtgcctgttttggtaaagaactggaagaaggaatgcgccaagactaggaagcagccggtgaaggttgtgtactcctccaaggatcctgcccagccgaaagagagttccaaggtggatgcgaatgaggaggtggaggctttgatcgtcaagtccccacagaaggagtggaacccctctctgtttaaggtgttatacaagacctttgggccctacttcctcatgagcttcttcttcaaggccatccacgacctgatgatgttttccgggccgcagatcttaaagttgctcatcaagttcgtgaatgacacgaaggccccagactggcagggctacttctacaccgtgctgctgtttgtcactgcctgcctgcagaccctcgtgctgcaccagtacttccacatctgcttcgtcagtggcatgaggatcaagaccgctgtcattggggctgtctatcggaaggccctggtgatcaccaattcagccagaaaatcctccacggtcggggagattgtcaacctcatgtctgtggacgctcagaggttcatggacttggccacgtacattaacatgatctggtcagcccccctgcaagtcatccttgctctctacctcctgtggctgaatctgggcccttccgtcctggctggagtggcggtgatggtcctcatggtgcccgtcaatgctgtgatggcgatgaagaccaagacgtatcaggtggcccacatgaagagcaaagacaatcggatcaagctgatgaacgaaattctcaatgggatcaaagtgctaaagctttatgcctgggagctggcattcaaggacaaggtgctggccatcaggcaggaggagctgaaggtgctgaagaagtctgcctacctgtcagccgtgggcaccttcacctgggtctgcacgccctttctggtggccttgtgcacatttgccgtctacgtgaccattgacgagaacaacatcctggatgcccagacagccttcgtgtctttggccttgttcaacatcctccggtttcccctgaacattctccccatggtcatcagcagcatcgtgcaggcgagtgtctccctcaaacgcctgaggatctttctctcccatgaggagctggaacctgacagcatcgagcgacggcctgtcaaagacggcgggggcacgaacagcatcaccgtgaggaatgccacattcacctgggccaggagcgaccctcccacactgaatggcatcaccttctccatccccgaaggtgctttggtggccgtggtgggccaggtgggctgcggaaagtcgtccctgctctcagccctcttggctgagatggacaaagtggaggggcacgtggctatcaagggctccgtggcctatgtgccacagcaggcctggattcagaatgattctctccgagaaaacatcctttttggatgtcagctggaggaaccatattacaggtccgtgatacaggcctgtgccctcctcccagacctggaaatcctgcccagtggggatcggacagagattggcgagaagggcgtgaacctgtctgggggccagaagcagcgcgtgagcctggcccgggccgtgtactccaacgctgacatttacctcttcgatgatcccctctcagcagtggatgcccatgtgggaaaacacatctttgaaaatgtgattggccccaaggggatgctgaagaacaagacgcggatcttggtcacgcacagcatgagctacttgccgcaggtggacgtcatcatcgtcatgagtggcggcaagatctctgagatgggctcctaccaggagctgctggctcgagacggcgccttcgctgagttcctgcgtacctatgccagcacagagcaggagcaggatgcagaggagaacggggtcacgggcgtcagcggtccagggaaggaagcaaagcaaatggagaatggcatgctggtgacggacagtgcagggaagcaactgcagagacagctcagcagctcctcctcctatagtggggacatcagcaggcaccacaacagcaccgcagaactgcagaaagctgaggccaagaaggaggagacctggaagctgatggaggctgacaaggcgcagacagggcaggtcaagctttccgtgtactgggactacatgaaggccatcggactcttcatctccttcctcagcatcttccttttcatgtgtaaccatgtgtccgcgctggcttccaactattggctcagcctctggactgatgaccccatcgtcaacgggactcaggagcacacgaaagtccggctgagcgtctatggagccctgggcatttcacaagggatcgccgtgtttggctactccatggccgtgtccatcggggggatcttggcttcccgctgtctgcacgtggacctgctgcacagcatcctgcggtcacccatgagcttctttgagcggacccccagtgggaacctggtgaaccgcttctccaaggagctggacacagtggactccatgatcccggaggtcatcaagatgttcatgggctccctgttcaacgtcattggtgcctgcatcgttatcctgctggccacgcccatcgccgccatcatcatcccgccccttggcctcatctacttcttcgtccagaggttctacgtggcttcctcccggcagctgaagcgcctcgagtcggtcagccgctccccggtctattcccatttcaacgagaccttgctgggggtcagcgtcattcgagccttcgaggagcaggagcgcttcatccaccagagtgacctgaaggtggacgagaaccagaaggcctattaccccagcatcgtggccaacaggtggctggccgtgcggctggagtgtgtgggcaactgcatcgttctgtttgctgccctgtttgcggtgatctccaggcacagcctcagtgctggcttggtgggcctctcagtgtcttactcattgcaggtcaccacgtacttgaactggctggttcggatgtcatctgaaatggaaaccaacatcgtggccgtggagaggctcaaggagtattcagagactgagaaggaggcgccctggcaaatccaggagacagctccgcccagcagctggccccaggtgggccgagtggaattccggaactactgcctgcgctaccgagaggacctggacttcgttctcaggcacatcaatgtcacgatcaatgggggagaaaaggtcggcatcgtggggcggacgggagctgggaagtcgtccctgaccctgggcttatttcggatcaacgagtctgccgaaggagagatcatcatcgatggcatcaacatcgccaagatcggcctgcacgacctccgcttcaagatcaccatcatcccccaggaccctgttttgttttcgggttccctccgaatgaacctggacccattcagccagtactcggatgaagaagtctggacgtccctggagctggcccacctgaaggacttcgtgtcagcccttcctgacaagctagaccatgaatgtgcagaaggcggggagaacctcagtgtcgggcagcgccagcttgtgtgcctagcccgggccctgctgaggaagacgaagatccttgtgttggatgaggccacggcagccgtggacctggaaacggacgacctcatccagtccaccatccggacacagttcgaggactgcaccgtcctcaccatcgcccaccggctcaacaccatcatggactacacaagggtgatcgtcttggacaaaggagaaatccaggagtacggcgccccatcggacctcctgcagcagagaggtcttttctacagcatggccaaagacgccggcttggtgtgagccccagagctggcatatctggtcagaactgcagggcctatatgccagcgcccagggaggagtcagtacccctggtaaaccaagcctcccacactgaaaccaaaacataaaaaccaaacccagacaaccaaaacatattcaaagcagcagccaccgccatccggtcccctgcctggaactggctgtgaagacccaggagagacagagatgcgaaccacc

SEQ ID NO: 36 - DNA, 4192 bp, Human
tccaagctcaaagaagcagaggccgctgttcgtttcctttaggtctttccactaaagtcggagtatcttcttccaagatttcacgtcttggtggccgttccaaggagcgcgaggtcgggatggatcttgaaggggaccgcaatggaggagcaaagaagaagaacttttttaaactgaacaataaaagtgaaaaagataagaaggaaaagaaaccaactgtcagtgtattttcaatgtttcgctattcaaattggcttgacaagttgtatatggtggtgggaactttggctgccatcatccatggggctggacttcctctcatgatgctggtgtttggagaaatgacagatatctttgcaaatgcaggaaatttagaagatctgatgtcaaacatcactaatagaagtgatatcaatgatacagggttcttcatgaatctggaggaagacatgaccaggtatgcctattattacagtggaattggtgctggggtgctggttgctgcttacattcaggtttcattttggtgcctggcagctggaagacaaatacacaaaattagaaaacagttttttcatgctataatgcgacaggagataggctggtttgatgtgcacgatgttggggagcttaacacccgacttacagatgatgtctccaagattaatgaaggaattggtgacaaaattggaatgttctttcagtcaatggcaacatttttcactgggtttatagtaggatttacacgtggttggaagctaacccttgtgattttggccatcagtcctgttcttggactgtcagctgctgtctgggcaaagatactatcttcatttactgataaagaactcttagcgtatgcaaaagctggagcagtagctgaagaggtcttggcagcaattagaactgtgattgcatttggaggacaaaagaaagaacttgaaaggtacaacaaaaatttagaagaagctaaaagaattgggataaagaaagctattacagccaatatttctataggtgctgctttcctgctgatctatgcatcttatgctctggccttctggtatgggaccaccttggtcctctcaggggaatattctattggacaagtactcactgtattttctgtattaattggggcttttagtgttggacaggcatctccaagcattgaagcatttgcaaatgcaagaggagcagcttatgaaatcttcaagataattgataataagccaagtattgacagctattcgaagagtgggcacaaaccagataatattaagggaaatttggaattcagaaatgttcacttcagttacccatctcgaaaagaagttaagatcttgaagggtctgaacctgaaggtgcagagtgggcagacggtggccctggttggaaacagtggctgtgggaagagcacaacagtccagctgatgcagaggctctatgaccccacagaggggatggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagccaatgcctatgactttatcatgaaactgcctcataaatttgacaccctggttggagagagaggggcccagttgagtggtgggcagaagcagaggatcgccattgcacgtgccctggttcgcaaccccaagatcctcctgctggatgaggccacgtcagccttggacacagaaagcgaagcagtggttcaggtggctctggataaggccagaaaaggtcggaccaccattgtgatagctcatcgtttgtctacagttcgtaatgctgacgtcatcgctggtttcgatgatggagtcattgtggagaaaggaaatcatgatgaactcatgaaagagaaaggcatttacttcaaacttgtcacaatgcagacagcaggaaatgaagttgaattagaaaatgcagctgatgaatccaaaagtgaaattgatgccttggaaatgtcttcaaatgattcaagatccagtctaataagaaaaagatcaactcgtaggagtgtccgtggatcacaagcccaagacagaaagcttagtaccaaagaggctctggatgaaagtatacctccagtttccttttggaggattatgaagctaaatttaactgaatggccttattttgttgttggtgtattttgtgccattataaatggaggcctgcaaccagcatttgcaataatattttcaaagattataggggtttttacaagaattgatgatcctgaaacaaaacgacagaatagtaacttgttttcactattgtttctagcccttggaattatttcttttattacatttttccttcagggtttcacatttggcaaagctggagagatcctcaccaagcggctccgatacatggttttccgatccatgctcagacaggatgtgagttggtttgatgaccctaaaaacaccactggagcattgactaccaggctcgccaatgatgctgctcaagttaaaggggctataggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtgctgggaagatcgctactgaagcaatagaaaacttccgaaccgttgtttctttgactcaggagcagaagtttgaacatatgtatgctcagagtttgcaggtaccatacagaaactctttgaggaaagcacacatctttggaattacattttccttcacccaggcaatgatgtatttttcctatgctggatgtttccggtttggagcctacttggtggcacataaactcatgagctttgaggatgttctgttagtattttcagctgttgtctttggtgccatggccgtggggcaagtcagttcatttgctcctgactatgccaaagccaaaatatcagcagcccacatcatcatgatcattgaaaaaacccctttgattgacagctacagcacggaaggcctaatgccgaacacattggaaggaaatgtcacatttggtgaagttgtattcaactatcccacccgaccggacatcccagtgcttcagggactgagcctggaggtgaagaagggccagacgctggctctggtgggcagcagtggctgtgggaagagcacagtggtccagctcctggagcggttctacgaccccttggcagggaaagtgctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagattgtgagggcagcaaaggaggccaacatacatgccttcatcgagtcactgcctaataaatatagcactaaagtaggagacaaaggaactcagctctctggtggccagaaacaacgcattgccatagctcgtgcccttgttagacagcctcatattttgcttttggatgaagccacgtcagctctggatacagaaagtgaaaaggttgtccaagaagccctggacaaagccagagaaggccgcacctgcattgtgattgctcaccgcctgtccaccatccagaatgcagacttaatagtggtgtttcagaatggcagagtcaaggagcatggcacgcatcagcagctgctggcacagaaaggcatctatttttcaatggtcagtgtccaggctggaacaaagcgccagtgaactctgactgtatgagatgttaaatactttttaatatttgtttagatatgacatttattcaaagttaaaagcaaacacttacagaattatgaagaggtatctgtttaacatttcctcagtcaagttcagagtcttcagagacttcgtaattaaaggaacagagtgagagacatcatcaagtggagagaaatcatagtttaaactgcattataaattttataacagaattaaag

SEQ ID NO: 37 - DNA, 3266 bp, Human
gcggtgcgggccgggcgggtgcattcaggccaaggcggggccgccgggatgctcagggttccggagccgcggcccggggaggcgaaagcggagggggccgcgccgccgaccccgtccaagccgctcacgtccttcctcatccaggacatcctgcgggacggcgcgcagcggcaaggcggccgcacgagcagccagagacagcgcgacccggagccggagccagagccagagccagagggaggacgcagccgcgccggggcgcagaacgaccagctgagcaccgggccccgcgccgcgccggaggaggccgagacgctggcagagaccgagccagaaaggcacttggggtcttatctgttggactctgaaaacacttcaggcgcccttccaaggcttccccaaacccctaagcagccgcagaagcgctcccgagctgccttctcccacactcaggtgatcgagttggagaggaagttcagccatcagaagtacctgtcagcccctgaacgggcccacctggccaagaacctcaagctcacggagacccaagtgaagatatggttccagaacagacgctataagactaagcgaaagcagctctcctcggagctgggagacttggagaagcactcctctttgccggccctgaaagaggaggccttctcccgggcctccctggtctccgtgtataacagctatccttactacccatacctgtactgcgtgggcagctggagcccagctttttggtaatgccagctcaggtgacaaccattatgatcaaaaactgccttccccagggtgtctcatatgaaaagcacaaggggccaaggtcagggagcaagaggtgtgcacaccaaaactattggagaattgcgtggaaatcttcagattcttcactggtgagacaatgaaacaacagagacagtgaaagttttaatacctaagtcattcccccagtgcatactgtagcgtcaagtttttgcttctggctacctgtttgaaggggagagagggaaaatcaagtggtattttccagcactttgtatgattttggatgagctgtacacccaaggattctgttctgcaactccatcctcctgtgtcactgaatatcaactctgaaagagcaaacctaacaggagaaaggacaaccaggatgaggatgtcaccaactgaattaaacttaagtccagaagcctcctgttggccttggaatatggccaaggctctctctgtccctgtaaaagagaggggcaaatagtctccaaagagaacgccctcatgctcagcacatatttgcatggaagggggagatgggtgggaggagatgaaaatatcagcttttcttattcctttttattccttttaaaatggtatgccaacttaagtatttacagggtggcccaaatagaacaagatgcactcgctgtgattttaagacaagctgtataaacagaactccactgcaagagggagggccgggccaggagaatctccgcttgtccaagacaggggcctaaggagggtctccacactgctgctaggggctgttgcatttttttattagtagaaagtggaaaggcctcttctcaacttttttcccttgggctggagaatttagaatcagaagtttcctggagttttcaggctatcatatatactgtatcctgaaaggcaacataattcttccttccctccttttaaaattttgtgttcctttttgcagcaattactcactaaagggcttcattttagtccagatttttagtctggctgcacctaacttatgcctcgcttatttagcccgagatccggtctttttttttttttttttttccgtctccccaaagctttatctgtcttgactttttaaaaaagtttgggggcagattctgaattggctaaaagacatgcatttttaaaactagcaactcttatttctttcctttaaaaatacatagcattaaatcccaaatcctatttaaagacctgacagcttgagaagggtcactactgcatttataggaccttctggtggttctgctgttacgtttgaagtctgacaatccttgagaatctttgcatgcagaggaggtaagaggtattggattttcacagagggaagaacacagcgcagaatgaaggcgcaggcttactgagctgtccagtggagggctcatgggtgggacatggaaaagaaggcagcctaggccctggggagcccagtccactgagcaagcaagggactgagtgagccttttgcaggaaaaggctaagaaaaaggaaaaccattctaaaacacaacaagaaactgtccaaatgctttgggaactgtgtttattgcctataatggggtccccaaaatgggtaacctagacttcagagagaatgagcagagagcaaaggagaaatctgggctgtccttccattttcattctgttatctcaggtgagctggtagaggggagacattagaaaaaaatgaaacaacaaaacaattactaatgaggtacgctgaggcctgggagtctcttgagctccacgacttcaaaattaaaatgagccatgagtcaaaccactgcaatccagcctgggcaacgagcaagacccagtctctactgttggtggcaaaattgccaacataagttaatagaaagttggccaatttcaccccattttctgtggtttgggctccacattgcaatgttcaatgccacgtgctgctgacaccgaccggagtactagccagcacaaaaggcagggtagcctgaattgctttctgctctttacatttcttttaaaataagcatttagtgctcagtccctactgagtactctttctctcccctcctctgaatttaattctttcaacttgcaatttgcaagggttacacatttcactgtgatgtatattgtgttgcaaaaaaaaaaaagtgtctttgtttaaaattacttggtttgtgaatccatcttgctttccccattggaactagtcattaacccatctctgaactggtagaaaaacatctgaagagctagtctatcagcatctgacaggtgaattggatggttctcagaaccatttcacccagacagcctgtttctatcctgtttaataaattagtttgggttctctacatgcataacaaaccctgctccaatctgtcacataaaagtctgtgacttgaagtttagtcagcacccccaccaaactttatttttctatgtgttttttgcaacatatgagtgttttgaaaataaagt

SEQ ID NO: 38 - DNA, 4530 bp, Human; identical to SEQ ID NO: 27

SEQ ID NO: 39 - DNA, 4139 bp, Human
ccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 40 - DNA, 3860 bp, Human
agggagccccagaaaagcggaagaatttagacgcacactgggtcgcatagggcatggagctggagaactataaacagcccgtggtgctgagagaggacaactgccgaaggcgccggaggatgaagccgcgcagtgctgcggccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccgctggtggacgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcagcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagtgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctaatactttaggctagaatcaaaaacaagttagtgttctatggtttaaattagcatagcaatcatcgaacttggatttcaaatgcaatagacattgtgaaagctggcatttcagaagtatagctcttttcctacctgaactcttccctggagaaaagatgttggcattgctgattgtttggttaagcaatgtccagtgctaggattatttgcaggtttggttttttctcatttgtctgtggcattggagaatattcttggtttaaacagactaatgacttccttattgtccctgatattttgactatcttactattgagtgcttctggaaattctttggaataattgatgacatctattttcatctgggtttagtctcaattttggttatctttgtgttcctcaagctctttaaagaaaaagatgtaatcgttgtaaaaaaaaaaaaaaaaaaaaaaaaaaaaaaaaaaaaaaaaaaaaaaaaaaaaaaaaaaaaaaaaaa

SEQ ID NO: 41 - DNA, 1446 bp, Human
attccgccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaactccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggtaccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatacgtg

SEQ ID NO: 42 - DNA, 2653 bp, Human
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 43 - DNA, 2365 bp, Human
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggttttcgaaacttacatattgatgaccagataactctcattcagtattcttggatgagcttaatggtgtttggtctaggatggagatcctacaaacacgtcagtgggcagatgctgtattttgcacctgatctaatactaaatgattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtga

SEQ ID NO: 44 - DNA, 591 bp, Human
cttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaaccggttttatactattgaaatcctaaaggtagaataatggaagccctgtctgtttgccacacccaggtgatttcctctaaagaaacttggctggaatttctgctgtggtctataaaataaacttcttaacatgcttaaaaaaaaaaaaaaa

SEQ ID NO: 45 - DNA, 1547 bp, Human
gggagtcatcatgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatatgatgaggatggtatggactgcatggacaatgagaggcggccgcatttccctcaattttcctactctgcaagtggacgagaataagtctctttcattctgctacttcactgtcatcttcaatttattactgaaaatgattcctggacatcaccagtcctagctcttacacatagcaggggca

SEQ ID NO: 46 - DNA, 2799 bp, Human
ctcacacaccctgaagacacagtgagttagcaccaccaccaggaattggccttttagctctgtgcctgtctccagtcaggctggaataagtctcctcatatttgcaagctcggccctcccctggaatctaaagcctcctcagccttctgagtcagcctgaaaggaacaggccgaactgctgtatgggctctactgccagtgtgacctcaccctctccagtcacccctcctcagttccagctatgagttcctgcaacttcacacatgccacctttgtgcttattggtatcccaggattagagaaagcccatttctgggttggcttccccctcctttccatgtatgtagtggcaatgtttggaaactgcatcgtggtcttcatcgtaaggacggaacgcagcctgcacgctccgatgtacctctttctctgcatgcttgcagccattgacctggccttatccacatccaccatgcctaagatccttgcccttttctggtttgattcccgagagattagctttgaggcctgtcttacccagatgttctttattcatgccctctcagccattgaatccaccatcctgctggccatggcctttgaccgttatgtggccatctgccacccactgcgccatgctgcagtgctcaacaatacagtaacagcccagattggcatcgtggctgtggtccgcggatccctcttttttttcccactgcctctgctgatcaagcggctggccttctgccactccaatgtcctctcgcactcctattgtgtccaccaggatgtaatgaagttggcctatgcagacactttgcccaatgtggtatatggtcttactgccattctgctggtcatgggcgtggacgtaatgttcatctccttgtcctattttctgataatacgaacggttctgcaactgccttccaagtcagagcgggccaaggcctttggaacctgtgtgtcacacattggtgtggtactcgccttctatgtgccacttattggcctctcagttgtacaccgctttggaaacagccttcatcccattgtgcgtgttgtcatgggtgacatctacctgctgctgcctcctgtcatcaatcccatcatctatggtgccaaaaccaaacagatcagaacacgggtgctggctatgttcaagatcagctgtgacaaggacttgcaggctgtgggaggcaagtgacccttaacactacacttctccttatctttattggcttgataaacataattatttctaacactagcttatttccagttgcccataagcacatcagtacttttctctggctggaatagtaaactaaagtatggtacatctacctaaaggactattatgtggaataatacatactaatgaagtattacatgatttaaagactacaataaaaccaaacatgcttataacattaagaaaaacaataaagatacatgattgaaaccaagttgaaaaatagcatatgccttggaggaaatgtgctcaaattactaatgatttagtgttgtccctactttctctctcttttttctttcttttttttttattatggttagctgtcacatacaacttttttttttttgagatggggtctcgctctgtcaccaggctggagtgcagtggcgcgatctcggctcactgcaacctccacatcccatgttgaagtaattcttctgcctcagcctcccgagtagctgggactagaggaacgtgccaccatgactggctaattttctgtattttttagtagagacagagtttcaccatgttggccaggatggtctcgatctcctgaccttgtgatccacccgcctcagcctcccaaagtgttgggattacaggtgtgaaccactgtgcccggcctgtgtacaactttttaaatagggaatatgatagcttcgcatggtggtgtgcacctatagcccccactgcctggaaagctgaggtgggagaatcgcttgagtccaggagtttgaggttacagtgatccacgatcgtaccactacactccagcctgggcaacggagcaagaccctgtctcaaagcataaaatggaataacatatcaaatgaaacagggaaaatgaagctgacaatttatggaagccagggcttgtcacagtctctactgttattatgcattacctgggaatttatataagcccttaataataatgccaatgaacatctcatgtgtgctcacaatgttctggcactattataagtgcttcacaggttttatgtgttcttcgtaactttatggagtaggtaccatttgtgtctctttattataagtgagagaaatgaagtttatattatcaaggggactaaagtcacacggcttgtgggcactgtgccaagatttaaaattaaatttgatggttgaatacagttacttaatgaccatgttatattgcttcctgtgtaacatctgccatttatttcctcagctgtacaaatcctctgttttctctctgttacacactaacatcaatggctttgtacttgtgatgagagataaccttgccctagttgtgggcaacacatgcagaataatcctgttttacagctgcctttcgtgatcttattgcttgcttttttccagattcagggagaatgttgttgtctatttgtctcttacatctccttgatcatgtcttcattttttaatgtgctctgtacctgtcaaaaattttgaatgtacaccacatgctattgtctgaacctgagtataagataaaataaaattttattttaaattttaaaaaaaaaaaaaaaaaa

SEQ ID NO: 47 - DNA, 2629 bp, Human
acttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgcca

SEQ ID NO: 48 - DNA, 4015 bp, Human
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 49 - DNA, 782 bp, Human
aggggccttagcgtgccgcatcgccgagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagtttcgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaactgaagcctgcacagtgtccaccctgttcccactcccatctttcttccggacaatgaaataaagagttaccacccagc

SEQ ID NO: 50 - DNA, 1075 bp, Human
cgcagcaaacacatccgtagaaggcagcgcggccgccgagagccgcagcgccgctcgcccgccgccccccaccccgccgccccgcccggcgaattgcgccccgcgcccctcccctcgcgcccccgagacaaagaggagagaaagtttgcgcggccgagcggggcaggtgaggagggtgagccgcgcgggaggggcccgcctcggccccggctcagcccccgcccgcgcccccagcccgccgccgcgagcagcgcccggaccccccagcggcggcccccgcccgcccagccccccggcccgccatgggcgccgcggcccgcaccctgcggctggcgctcggcctcctgctgctggcgacgctgcttcgcccggccgacgcctgcagctgctccccggtgcacccgcaacaggcgttttgcaatgcagatgtagtgatcagggccaaagcggtcagtgagaaggaagtggactctggaaacgacatttatggcaaccctatcaagaggatccagtatgagatcaagcagataaagatgttcaaagggcctgagaaggatatagagtttatctacacggccccctcctcggcagtgtgtggggtctcgctggacgttggaggaaagaaggaatatctcattgcaggaaaggccgagggggacggcaagatgcacatcaccctctgtgacttcatcgtgccctgggacaccctgagcaccacccagaagaagagcctgaaccacaggtaccagatgggctgcgagtgcaagatcacgcgctgccccatgatcccgtgctacatctcctccccggacgagtgcctctggatggactgggtcacagagaagaacatcaacgggcaccaggccaagttcttcgcctgcatcaagagaagtgacggctcctgtgcgtggtaccgcggcgcggcgccccccaagcaggagtttctcgacatcgaggacccataagcaggcctccaacgcccctgtggccaactgcaaaaaaagcctccaagggtttcgactggtccagctctgacatcccttcctggaaacagcatgaataaaacactcatccc

SEQ ID NO: 51 - DNA, 5510 bp, Human
  misc_feature (2780)..(2780): n is a, c, g, or t
tagagacggggtctcgctatgttgcccaggctggtctccaactcctaggctccagcgatcctcccgcctcggcctcccaatgtgctgcgaatacagactccagccaccgcacacagcctacttttatttctttgaaaaatgaattcgagggtaaaggggcggggttgaggcagatgccagaatctgttcgcttcaaccaagcagccaggctgcctgtccagaaagccggcactcagtttcctcaggaaaacgaagctaaggctcccattcccctcgctaacaacgtcagaacagaggacagtttttagatttcagggatcttaaatagattggcagttcctggagaataaacatcctttgcttttctcctgcacacttttgcctcaggccaccccttcccgcttccaaagcccatctcttccaagctttccgcacgagaaaacaagtgagcccttctcattggccagattccctgtcaatctctccgctatgacgccgagtggtgccttttgaagcctctctagtcccgcctccctaacctgattggtttattcaaacaaaccccggccaactcagccgttcataggtggatataaaaggcaagctacgattggttcttctggacggagacggtgagagcgagtcagggattggctggtctgcttcgggcgggctaaaggaaggttcaagtggagctctcctaaccgacgcgcgtctgtggagaagcggcttggtcgggggtggtctcgtggggtcctgcctgtttagtcgctttcagggttcttgagccccttcacgaccgtcaccatggaagtgtcaccattgcaggtacggctcgcgggggggactggcggtggagcctccgcgcggcccgggcatctctctggccgcccgtgacgggtgaagctctggggctgcggtcaggccggcgaccggcttgggagcccatattctccattcccggttccggggtgatcgtggagaggcggaagccccttctggtgctagtagtgaagtatgacccggcttccagggtgtcgtgcgttgttgccctgtttctaggggcaggagtccgttggtcccgtagtggatccatgttacagcggcccgggtgcgacgttattgagtcgcgcgtacagatgctttcccctcctgcccgctgcttgaaaacggtcttgaatgtccccgatcttggaaagggcagagcctagcacagtgtttctctggagcagctggccagcttacaaaagcgcccccttcctgagttcacaacgctcaggtggccaagggattgaacggatagccgcccaaactgctgctgccgaactagaattatttacggtgtcgtgtgctcgggacaccgtaaataatttataataataggcactcggtaaataatgtatgcaggaactgaaagaagtgggtgtattttattgatgacatctaccatgttgctcaagaaaagaggtttccagacagacccacttggtgagataatatgtgtctgtttaacctgacacctggggacctggccgcagcgaatttaaattgtcctatttgtacaaaactggcaactccaccactgcctactaacagattgcaatgcatgttgacatctcactatggtaggcattttgttcctagtttagggtacacttatgattaaaaattcccttctttctctctttttttttaattgctagcctgtaaatgaaaatatgcaagtcaacaaaataaagaaaaatgaagatgctaagaaaagactgtctgttgaaagaatctatcaaaagaaaacacaattggaacatattttgctccgcccagacacctacattggttctgtggaattagtgacccaggtaaaatcaagctcatcaaaggcagttaacctagtagttgaaatggatactcttctttctgtacctttcatgtactgagtgtgacctttctgtatctctcatgtactgagtgtcactggaactggcctaaatatggtaatacttaatatgaacactttatggtaatcatgataacagaacaatgaatatagaataaaaataacggtatttctttgccttatttctagcaaatgtgggtttacgatgaagatgttggcattaactatagggaagtcacttttgttcctggtttgtacaaaatctttgatgagattctaggtgagtaaatcctttaatataaagatctggtcaaatctttcataagaagtatcattccaaataaaaatttaaaaaagataaaagtaaaaacaaaaagtatcatttttaccaaacctgctgtagccagggctcctgtcatttcaatcagttgataaaggtgagctggtttcctggagcccaggaggttgagcttctcgtagaaacagtactatggacaaaaattgctaaattaacaccttgttaatgccactcctgtgctgtttgggaacttttttttctatatcttacttagagttacagaatgttttttaaaacactaatttcttacaaatgtatattttattttctttcagttaatgctgcggacaacaaacaaagggacccaaaaatgtcttgtattagagtcacaattgatccgtacgtcttttgcttagtttttgttatgtagctcgttgctattgttagctgcatgtattttgattactggtgaagttggcttttcctaaacagaagttgagaaaagtacacaaaagcagaatgctaaaatgaaaaacctatggatacccaccacctaaatccaacagttgccaacgttttgtccanattttctccanctttttttgaattatttaatttttttaaaggaaggggtcttgctattttgcctgggctggtcttaaaactcctggcctgaagcaatcctccagtgttggcattatgggtgtgtgccatggcacgcggcctatttgctctggatcatttcaaaataaattacaggactcacgacatctaatccctaaatatttgagcaagcaccttcagtaattgctttaagtattctggcatctggagttttaactttttctcatgatgctattcaaacagtaaaccagtaggtggtggtagagaataatttgatctgacatttctgcttataaatgcggggtgtcccttagtgggtgatcagggtgcttatttcacttttttgttagtctgattgattatgacaaagtatacctggattttcctaaggactcaatatcatagtctttaaaaaatgttgagctagggccaggcacggtggctcatgcctgtgatcccagcactttgggaggccgatgcgggcggatcacaaggtcaggagatcaagaccatcctggctaacacggtgaaaccctgtctccactaaaaatacaaaaaattagcccggtgtggtgatggacacctntagtcccagctattcaggaggctgaagtgggaggattgcttgagcccaggaggtcaagactgcantaagctgtgatcctgccattacagtgcagcctgggtgacagagagagatgctgtctcaaaaaaggagtgggagtggtgtataataatgtttccagattgtccttttctttccattgggcataaatcatttccctctgtttatcctaacattttaaaattttgtattttttcaacattatcagtgtctattcaggtgttggaaaaggtttttttttcctcttgactcctatttcaggaatgtcaggaactaagcatggtgggaaatgttcctgctgaatttcagagggtatatatgaaaagtgccaaaaaaattgagggctgggtatggtggctcacatctataaccccaacactttgagcagttctcctgcttcagcctcccaagtagctggaatcacaggcatgcgctaccatgcctggctaattttttttatttttagtagagacggtttcaacatgttggccagaactcctggcctcaagtgatttgccctcctcagcctcccaaagtgctgggattacaggcacaggccaccgcgcccagcctgaatatacatattttagaaataatagtatttaaatatttttaaatgttaagcattcgatttgatgtgttgtaagatacaattttcaatcttttttttcttagggaaaacaatttaattagtatatggaataatggaaaaggtattcctgttgttgaacacaaagttgaaaagatgtatgtcccagctctcatatttggacagctcctaacttctagtaactatgatgatgatgaaaagaaagtgacaggtagagtattgaggggaaataacatatttgttgctaaaaatatatatatttaaatgactgtctgtggcatgagggttaaagatatggaaataaatctctataattgaatagctctgccagtgattaagaaataaagctgtcaatgagatagtaacaataaaatagtgtttcatatttatttgcccaggtggtcgaaatggctatggagccaaattgtgtaacatattcagtaccaaatttactgtggaaacagccagtagagaatacaagaaaatgttcaaacaggcaagtaaataagtgtcttgtaccttaatgataaatggtagtagtatagccatttataatggcattaatgattggtttaatttaacataatttataagctattgaagtatggaaaattataagcatatatattaggttattaggactcataaatttatgttatttacttccagtttgtgagatgacttgaatttttcatgtttccttattctttacttccatagacatggatggataatatgggaagagctggtgagatggaactcaagcccttcaatggagaagattatacatgtatcacctttcagcctgatttgtctaagtttaaaatgcaaagcctggacaaagatattgttgcactaatggtcagaagagcatatgatattgctggatccaccaaagatgtcaaagtctttcttaatggaaataaactgccagtgagtattttcctggatgttaaggataataagggattttgtaatcattgtcaagtgcaaaattgaattttttcccctcccatatgtttttgtttgtttgtttgtttgtttgtttgagacagagtctcacactgttgcccgggctggagtgcagtggcacgatctcggctcaccgcaacctccacctcccaggttcacgcaattctcctgcctcagcctcccaagtagctgggattacaggtgcctgccaccacacctggctaattttttgtattttcagtagagacaggttcactatgttggccaggctggtctcgaacaccagacctcatgatccacccgtcttggcctcccaaagtgctgggattacaggcatgagccactgcacctggccaaccatgtgtatttcttaccacttctcacatatgttcttgaaaagagaatggtatgccacattttttaatccgctcattttaaacttaccgatggatttccttctcaaagaaacaccaaaaataaatat

SEQ ID NO: 52 - DNA, 5189 bp, Human
atggccaagtcgggtggctgcggcgcgggagccggcgtgggcggcggcaacggggcactgacctgggtgaacaatgctgcaaaaaaagaagagtcagaaactgccaacaaaaatgattcttcaaagaagttgtctgttgagagagtgtatcagaagaagacacaacttgaacacattcttcttcgtcctgatacatatattgggtcagtggagccattgacgcagttcatgtgggtgtatgatgaagatgtaggaatgaattgcagggaggttacctttgtgccaggtttatacaagatctttgatgaaattttggttaatgctgctgacaataaacagagggataagaacatgacttgtattaaagtttctattgatcctgaatctaacattataagcatttggaataatgggaaaggcattccagtagtagaacacaaggtagagaaagtttatgttcctgctttaatttttggacagcttttaacatccagtaactatgatgatgatgagaaaaaagttacaggtggtcgtaatggttatggtgcaaaactttgtaatattttcagtacaaagtttacagtagaaacagcttgcaaagaatacaaacacagttttaagcagacatggatgaataatatgatgaagacttctgaagccaaaattaaacattttgatggtgaagattacacatgcataacattccaaccagatctgtccaaatttaagatggaaaaacttgacaaggatattgtggccctcatgactagaagggcatatgatttggctggttcgtgtagaggggtcaaggtcatgtttaatggaaagaaattgcctgtaaatggatttcgcagttatgtagatctttatgtgaaagacaaattggatgaaactggggtggccctgaaagttattcatgagcttgcaaatgaaagatgggatgtttgtctcacattgagtgaaaaaggattccagcaaatcagctttgtaaatagtattgcaactacaaaaggtggacggcacgtggattatgtggtagatcaagttgttggtaaactgattgaagtagttaagaaaaagaacaaagctggtgtatcagtgaaaccatttcaagtaaaaaaccatatatgggtttttattaattgccttattgaaaatccaacttttgattctcagactaaggaaaacatgactctgcagcccaaaagttttgggtctaaatgccagctgtcagaaaaattttttaaagcagcctctaattgtggcattgtagaaagtatcctgaactgggtgaaatttaaggctcagactcagctgaataagaagtgttcatcagtaaaatacagtaaaatcaaaggtattcccaaactggatgatgctaatgatgctggtggtaaacattccctggagtgtacactgatattaacagagggagactctgccaaatcactggctgtgtctggattaggtgtgattggacgagacagatacggagtttttccactcaggggcaaaattcttaatgtacgggaagcttctcataaacagatcatggaaaatgctgaaataaataatattattaaaatagttggtctacaatataagaaaagttacgatgatgcagaatctctgaaaaccttacgctatggaaagattatgattatgaccgatcaggatcaagatggttctcacataaaaggcctgcttattaatttcatccatcacaattggccatcacttttgaagcatggttttcttgaagagttcattactcctattgtaaaggcaagcaaaaataagcaggaactttccttctacagtattcctgaatttgacgaatggaaaaaacatatagaaaaccagaaagcctggaaaataaagtactataaaggattgggtactagtacagctaaagaagcaaaggaatattttgctgatatggaaaggcatcgcatcttgtttagatatgctggtcctgaagatgatgctgccattaccttggcatttagtaagaagaagattgatgacagaaaagaatggttaacaaattttatggaagaccggagacagcgtaggctacatggcttaccagagcaatttttatatggtactgcaacaaagcatttgacttataatgatttcatcaacaaggaattgattctcttctcaaactcagacaatgaaagatctataccatctcttgttgatggctttaaacctggccagcggaaagttttatttacctgtttcaagaggaatgataaacgtgaagtaaaagttgcccagttggctggctctgttgctgagatgtcggcttatcatcatggagaacaagcattgatgatgactattgtgaatttggctcagaactttgtgggaagtaacaacattaacttgcttcagcctattggtcagtttggaactcggcttcatggtggcaaagatgctgcaagccctcgttatattttcacaatgttaagcactttagcaaggctactttttcctgctgtggatgacaacctccttaagttcctttatgatgataatcaacgtgtagagcctgagtggtatattcctataattcccatggttttaataaatggtgctgagggcattggtactggatgggcttgtaaactacccaactatgatgctagggaaattgtgaacaatgtcagacgaatgctagatggcctggatcctcatcccatgcttccaaactacaaaaactttaaaggcacgattcaagaacttggtcaaaaccagtatgcagtcagtggtgaaatatttgtagtggacagaaacacagtagaaattacagagcttccagttagaacttggacacaggtatataaagaacaggttttagaacctatgctaaatggaacagataaaacaccagcattaatttctgattataaagaatatcatactgacacaactgtgaaatttgtggtgaaaatgactgaagagaaactagcacaagcagaagctgctggactgcataaagtttttaaacttcaaactactcttacttgtaattccatggtactttttgatcatatgggatgtctgaagaaatatgaaactgtgcaagacattctgaaagaattctttgatttacgattaagttattacggtttacgtaaggagtggcttgtgggaatgttgggagcagaatctacaaagcttaacaatcaagcccgtttcattttagagaagatacaagggaaaattactatagagaataggtcaaagaaagatttgattcaaatgttagtccagagaggttatgaatctgacccagtgaaagcctggaaagaagcacaagaaaaggcagcagaagaggatgaaacacaaaaccagcatgatgatagttcctccgattcaggaactccttcaggcccagattttaattatattttaaatatgtctctgtggtctcttactaaagaaaaagttgaagaactgattaaacagagagatgcaaaagggcgagaggtcaatgatcttaaaagaaaatctccttcagatctttggaaagaggatttagcggcatttgttgaagaactggataaagtggaatctcaagaacgagaagatgttctggctggaatgtctggaaaagcaattaaaggtaaagttggcaaacctaaggtgaagaaactccagttggaagagacaatgccctcaccttatggcagaagaataattcctgaaattacagctatgaaggcagatgccagcaaaaagttgctgaagaagaagaagggtgatcttgatactgcagcagtaaaagtggaatttgatgaagaattcagtggagcaccagtagaaggtgcaggagaagaggcattgactccatcagttcctataaataaaggtcccaaacctaagagggagaagaaggagcctggtaccagagtgagaaaaacacctacatcatctggtaaacctagtgcaaagaaagtgaagaaacggaatccttggtcagatgatgaatccaagtcagaaagtgatttggaagaaacagaacctgtggttattccaagagattctttgcttaggagagcagcagccgaaagacctaaatacacatttgatttctcagaagaagaggatgatgatgctgatgatgatgatgatgacaataatgatttagaggaattgaaagttaaagcatctcccataacaaatgatggggaagatgaatttgttccttcagatgggttagataaagatgaatatacattttcaccaggcaaatcaaaagccactccagaaaaatctttgcatgacaaaaaaagtcaggattttggaaatctcttctcatttccttcatattctcagaagtcagaagatgattcagctaaatttgacagtaatgaagaagattctgcttctgttttttcaccatcatttggtctgaaacagacagataaagttccaagtaaaacggtagctgctaaaaagggaaaaccgtcttcagatacagtccctaagcccaagagagccccaaaacagaagaaagtagtagaggctgtaaactctgactcggattcagaatttggcattccaaagaagactacaacaccaaaaggtaaaggccgaggggcaaagaaaaggaaagcatctggctctgaaaatgaaggcgattataaccctggcaggaaaacatccaaaacaacaagcaagaaaccgaagaagacatcttttgatcaggattcagatgtggacatcttcccctcagacttccctactgagccaccttctctgccacgaaccggtcgggctaggaaagaagtaaaatattttgcagagtctgatgaagaagaagatgatgttgattttgcaatgtttaattaagtgcccaaagagcacaaacatttttcaacaaatatcttgtgttgtccttttgtcttctctgtctcagacttttgtacatctggcttattttaatgtgatgatgtaattgacggttttttattattgtggtaggccttttaacattttgttcttacacatacagttttatgctcttttttactcattgaaatgtcacgtactgtctgattggcttgtagaattgttatagactgccgtgcattagcacagattttaattgtcatggttacaaactacagacctgctttttgaaatgaaatttaaacattaaaaatggaactgtg

SEQ ID NO: 53 - DNA, 2805 bp, Human; identical to SEQ ID NO: 20

SEQ ID NO: 54 - DNA, 1536 bp, Human
ggggggggggggaccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagcttgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttatagttgttttatatgttgctataataaagaagtgttctgc

SEQ ID NO: 55 - DNA, 1723 bp, Human
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgg

SEQ ID NO: 56 - DNA, 2304 bp, Human
gtccccgcagcgccgtcgcgccctcctgccgcaggccaccgaggccgccgccgtctagcgccccgacctcgccaccatgagagccctgctggcgcgcctgcttctctgcgtcctggtcgtgagcgactccaaaggcagcaatgaacttcatcaagttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctatgtgctgctgacccccaatggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtggcaccaccagggtgaacgacaatagctttaccctcacggataggcctgggtgctggctgcccagaccctctggccaggatggaggggtggtcctgactcaacatgttactgaccagcaacttgtctttttctggactgaagcctgcaggagttaaaaagggcagggcatctcctgtgcatgggctcgaagggagagccagctcccccgaccggtgggcatttgtgaggcccatggttgagaaatgaataatttcccaattaggaagtgtaagcagctgaggtctcttgagggagcttagccaatgtgggagcagcggtttggggagcagagacactaacgacttcagggcagggctctgatattccatgaatgtatcaggaaatatatatgtgtgtgtatgtttgcacacttgttgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtacttattctgcagcatgacctgtgaccagcactgtctcagtttcactttcacatagatgtccctttcttggccagttatcccttccttttagcctagttcatccaatcctcactgggtggggtgaggaccactccttacactgaatatttatatttcactatttttatttatatttttgtaattttaaataaaagtgatcaataaaatgtgatttttctga

SEQ ID NO: 57 - DNA, 1743 bp, Human
cagtatccctcctgacaaaactaacaaaaatcctgttagccaaataatcagccacattcatatttaccgtcaaagtttttatcctcattttacagcagtggagagcgattgccccgggtcccacgttaggaagagagagaactgggatttgcacccaggcaatctggggacagagctgtgatcacaactccatgagtcagggccgagccagccccttcaccaccagccggccgcgccccgggaaggaagtttgtggcggaggaggttcgtacgggaggagggggaggcgcccacgcatctggggctgactcgctctttcgcaaaacgtctgggaggagtccctggggccacaaaactgcctccttcctgaggccagaaggagagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggcaatgggagagctcttgttattattaatattgttgccgctgttgtgttgttgttattaattaatattcatattatttattttatacttacataaagattttgtaccagtgg

SEQ ID NO: 58 - DNA, 45 bp, Human
tctagtcgacggccagtgaattgtaatacgactcactatagggcg

SEQ ID NO: 59 - DNA, 22 bp, Human
tctagtcgacggccagtgaatt

SEQ ID NO: 60 - DNA, 20 bp, Human
aagacctacttcccgcactt

SEQ ID NO: 61 - DNA, 20 bp, Human
tatttggaggtcagcacggt

SEQ ID NO: 62 - DNA, 23 bp, Human
atctctgtgcaagtgcccaagat

SEQ ID NO: 63 - DNA, 25 bp, Human
caggaacatgttcatgacagactgt

SEQ ID NO: 64 - DNA, 23 bp, Human
gcaagagtgacagtggattgcat

SEQ ID NO: 65 - DNA, 23 bp, Human
ctaatggtggccaactggagact

SEQ ID NO: 66 - DNA, 25 bp, Human
agtcactgccttccaagtgcagcaa

SEQ ID NO: 67 - DNA, 27 bp, Human
ggaaacctgaaggctgatttgaagcag

SEQ ID NO: 68 - DNA, 22 bp, Human
gtggtttgagctcggcctatgg

SEQ ID NO: 69 - DNA, 21 bp, Human
ccagtgctaccctgcatagcg

SEQ ID NO: 70 - DNA, 28 bp, Human
gacggtagagttctttcatctacggttg

SEQ ID NO: 71 - DNA, 28 bp, Human
ggaaaccacaaaacaccttgtagacacc

SEQ ID NO: 72 - DNA, 24 bp, Human
accttgagtcagagctggcacaga

SEQ ID NO: 73 - DNA, 24 bp, Human
gcttctgctggcttaatgcctcag

SEQ ID NO: 74 - DNA, 22 bp, Human
catatccaggcgctgatcagcg

SEQ ID NO: 75 - DNA, 22 bp, Human
caaaggacagcagaagccccag

SEQ ID NO: 76 - DNA, 25 bp, Human
gtaagcctgggatgtgaagcaaagg

SEQ ID NO: 77 - DNA, 26 bp, Human
gaaccctaaagtggctcacaagagtg

SEQ ID NO: 78 - DNA, 26 bp, Human
cctgtaacctgactggttaacagcag

SEQ ID NO: 79 - DNA, 23 bp, Human
ggctctgactgatctgggagtca

SEQ ID NO: 80 - DNA, 23 bp, Human
gagatgcatagggaactcaatgc

SEQ ID NO: 81 - DNA, 23 bp, Human
acgatggagtccaagttctggat

SEQ ID NO: 82 - DNA, 21 bp, Human
agcaggtgcctgagacacaga

SEQ ID NO: 83 - DNA, 22 bp, Human
tcgagcatcccgctggattctt

SEQ ID NO: 84 - DNA, 22 bp, Human
ggaagctggctcacttgctgaa

SEQ ID NO: 85 - DNA, 22 bp, Human
gaagcacgtgggcattcagcat

SEQ ID NO: 86 - DNA, 23 bp, Human
tcgttggaagaggaacagcactg

SEQ ID NO: 87 - DNA, 23 bp, Human
agcctggatactgacaccattgc

SEQ ID NO: 88 - DNA, 20 bp, Human
cacaccatgcaggatgacat

SEQ ID NO: 89 - DNA, 20 bp, Human
gcattccacaaggttctcag

SEQ ID NO: 90 - DNA, 21 bp, Human
agtgtccaggctggaacaaag

SEQ ID NO: 91 - DNA, 23 bp, Human
ctccacttgatgatgtctctcac

SEQ ID NO: 92 - DNA, 22 bp, Human
ctcccagcactgctacgcaggc

SEQ ID NO: 93 - DNA, 24 bp, Human
gacataagaaagagaaggtgtggt

SEQ ID NO: 94 - DNA, 24 bp, Human
ctcctcctgcactgctatgcagat

SEQ ID NO: 95 - DNA, 29 bp, Human
acaccaaatgctgtcgtacactgtatgca

SEQ ID NO: 96 - DNA, 20 bp, Human
ctacaatcccatggtgctca

SEQ ID NO: 97 - DNA, 20 bp, Human
acacagttccatcagaccag

SEQ ID NO: 98 - DNA, 23 bp, Human
actgctggctgccttagaacctt

SEQ ID NO: 99 - DNA, 23 bp, Human
gagaagagactcggtagggacat

SEQ ID NO: 100 - DNA, 23 bp, Human
cgtcttccagtaccgagagaaag

SEQ ID NO: 101 - DNA, 23 bp, Human
tgtatccggcaaactggctcctt

SEQ ID NO: 102 - DNA, 23 bp, Human
ggtgatcgtcttggacaaaggag

SEQ ID NO: 103 - DNA, 23 bp, Human
tcttcacagccagttccaggcag

SEQ ID NO: 104 - DNA, 23 bp, Human
aacggtggcagcagcctctctta

SEQ ID NO: 105 - DNA, 25 bp, Human
gcttccacacactgagaagtgtccg

SEQ ID NO: 106 - DNA, 23 bp, Human
ggaagttcagccatcagaagtac

SEQ ID NO: 107 - DNA, 20 bp, Human
ggtatgggtagtaaggatag

SEQ ID NO: 108 - DNA, 23 bp, Human
ctgcctcagcctccggagtagct

SEQ ID NO: 109 - DNA, 23 bp, Human
gtggggtgaaaatgcagatgtgc

SEQ ID NO: 110 - DNA, 25 bp, Human
cagaactgtgcaaattgcagccgtc

SEQ ID NO: 111 - DNA, 26 bp, Human
agaccacagcagaaattccagccaag

SEQ ID NO: 112 - DNA, 23 bp, Human
tgaagcactgagcagaagctgga

SEQ ID NO: 113 - DNA, 23 bp, Human
gagggttgtctggaggacttcaa

SEQ ID NO: 114 - DNA, 26 bp, Human
acaccgctttggaaacagccttcatc

SEQ ID NO: 115 - DNA, 26 bp, Human
gtactgatgtgcttatgggcaactgg

SEQ ID NO: 116 - DNA, 23 bp, Human
agttcagtgagagactccaggac

SEQ ID NO: 117 - DNA, 23 bp, Human
ctgcactgtgaaggctgcaacat

SEQ ID NO: 118 - DNA, 22 bp, Human
agcacacctgccgtcttcactt

SEQ ID NO: 119 - DNA, 22 bp, Human
ggcacacctttggtcactccaa

SEQ ID NO: 120 - DNA, 23 bp, Human
ccaagacctacactgttggctgt

SEQ ID NO: 121 - DNA, 23 bp, Human
actgtgcaggcttcagttccact

SEQ ID NO: 122 - DNA, 22 bp, Human
tgggctgcgagtgcaagatcac

SEQ ID NO: 123 - DNA, 22 bp, Human
ctgcttatgggtcctcgatgtc

SEQ ID NO: 124 - DNA, 23 bp, Human
gccatccacttctgatgattctg

SEQ ID NO: 125 - DNA, 22 bp, Human
accagtcttgggcttggtaaga

SEQ ID NO: 126 - DNA, 22 bp, Human
aagcccaagagagccccaaaac

SEQ ID NO: 127 - DNA, 22 bp, Human
tggcagagaaggtggctcagta

SEQ ID NO: 128 - DNA, 25 bp, Human
tgttgctactctggtgtgtcccaag

SEQ ID NO: 129 - DNA, 25 bp, Human
ctgggattcaaaggaggtacagctc

SEQ ID NO: 130 - DNA, 23 bp, Human
tgggctgtgagtgtaagtgtgag

SEQ ID NO: 131 - DNA, 23 bp, Human
cacccagtgaggattggatgaac

(19) United States
(12) Patent Application Publication
(10) Pub. No.: US 2006/0008807 A1
(43) Pub. Date: Jan. 12, 2006

(54) Multiparameter analysis of comprehensive nucleic acids and morphological features on the same sample

(76) Inventors: Shawn Mark O'Hara, Ambler, PA (US); Daniel Zweitzig, Warminster, PA (US); Brad Foulk, Royersford, PA (US)

Correspondence Address: IMMUNICON CORPORATION, 3401 MASONS MILL ROAD, SUITE 100, HUNTINGDON VALLEY, PA, 19006, US

(21) Appl. No.: 10/826,585

(22) Filed: Apr. 16, 2004

(30) Foreign Application Priority Data
Aug. 23, 2002 (WO) PCT/US02/26867

(51) Int. Cl. C12Q 1/68

(52) U.S. Cl. 435/6

(57) ABSTRACT

A highly sensitive assay is disclosed which utilizes a method for gene specific primed amplification of mRNA libraries from rare cells and rare transcripts found in blood. The assay allows detection of rare mRNA (10 copies/cell) found in 1 to 10 cells isolated through immunomagnetic enrichment. The assay is an improvement over multiplex PCR and allows efficient detection of rare coding sequences for circulating carcinoma cells in the blood. The methods are useful in profiling of cells isolated from tissues or body fluids and serves as an adjunct to clinical diagnosis of diverse carcinomas including early stage detection and classification of circulating tumor cells. Monitoring of nucleic acid and protein profiles of cells either in conventional or microarray formats, facilitates management of therapeutic intervention including staging, monitoring response to therapy, confirmation of remission and detection of regression.

### PRIORITY INFORMATION

This application claims priority under 35 U.S.C. §119(e) to U.S. Provisional Applications, No. 60/369,945 (filed Apr. 4, 2002) and 60/330,669 (filed Oct. 26, 2001), and PCT/US02/26867 (filed Aug. 23, 2002).

### BACKGROUND OF THE INVENTION

1. Field of the Invention

This invention relates generally to gene specific amplification, analysis and profiling of cytosolic biomolecules useful in the fields of oncology and diagnostic testing. The invention is particularly useful in such fields as cancer screening, selecting and monitoring for chemotherapy treatment, or cancer recurrence. More specifically, the present invention provides methods, apparatus, and kits to facilitate comprehensive analysis of mRNA and DNA from tumor cells, or other rare cells from biological samples while simultaneously maintaining cell integrity for enumeration and morphological image analysis. To accomplish this, the invention also provides methods that permit the analysis of soluble cytosolic biomolecules releasable from a cell, such as a tumor cell, by means of permeabilizing reagents for determining expression profiles of the released nucleic acids, while still maintaining the morphological and antigenic characteristics of cells for subsequent or parallel multiparameter flowcytometric, image, and immunocytochemical analyses (see U.S. Pat. No. 6,365,362). The invention also provides methods that enable the same comprehensive analyses using stabilized samples from aldehyde and aldehyde-urea derivative based fixatives.

2. Description of Related Art

Any given cell will express only a fraction of the total number of genes present in its genome. A portion of the total number of genes that are expressed determine aspects of cell function such as development and differentiation, homeostasis, cell cycle regulation, aging, apoptosis, etc. Alterations in gene expression decide the course of normal cell development and the appearance of disease states, such as cancer. The expression of specific genes will have a profound effect on the nature of any given cell. Accordingly, the methods of analyzing gene expression, as such as those provided by the present invention, are important in basic molecular biological research and in tumor biology. Identification of specific genes, especially rare genes, can provide a key to diagnosis, prognosis and treatment for a variety of diseases that reflect these expression levels (Levsky, et al., Single-Cell Gene Expression Profiling, Science, 297:836-840, (2002)).

Differential gene expression is a commonly used method of assessing gene expression in a cell. In particular, cDNA microarray analysis compares cDNA target sequence levels obtained from cells or organs from healthy and diseased individuals. These targets are then hybridized to a set of probe fragments immobilized on a membrane. Differences in the resultant hybridization pattern are then detected and related to differences in gene expression of the two sources (U.S. Pat. No. 6,383,749). This procedure requires slow and time-consuming analysis of several hundred thousand gene-specific probes. In addition, competing events such as interactions between non-complementary target sequences nonspecific binding between target and probe and secondary structures in target sequences may interfere with hybridization resulting in a decline in the signal-to-noise.

Gene specific primer sets have been described in assaying differential expression (U.S. Pat. No. 5,994,076 and U.S. Pat. No. 6,352,829). Here, gene specific primer sets were used to specifically amplify mRNA library subsets in complex libraries achieving a cDNA array signal improvement when compared to whole library labeling amplification. The focus of this type of analysis was to compare sample array expression profiles as part of gene discovery research, not development of methods for practical cellular RNA analysis with utility in diagnostics.

Hence while gene specific primer sets have been used to selectively amplify a specific subset of mRNA from an mRNA library, there exists a clear need to reduce the signal-to-noise ratio in an amplification process which is especially applicable in rare cell detection for diagnostic therapy to encompass both quantitative and qualitative analysis.

It is now generally accepted that the presence of circulating tumor cells (CTC) in a patient's blood provides an early detection system in assessing the need for therapeutic intervention.

Highly sensitive assays to allow accurate enumeration of circulating carcinoma cells have shown that the peripheral blood tumor cell load correlate with disease state (Terstappen et al., Peripheral Blood Tumor Cell Load Reflects the Clinical Activity of the Disease in Patients with Carcinoma of the Breast, International J. of Oncology., 17:573-578, 2000).

Additionally, classification of cell type and origin would provide a more comprehensive platform for treatment. Emerging treatment for several cancers such as Diffuse Large B Cell Lymphoma (DLBCL) is based upon two different disease types correlating to a clinical prognosis (Rosenwald, et al., Use of Molecular Profiling to Predict Survival After Chemotherapy after Diffuse Large-B Cell Lymphoma, New England Journal of Medicine, 346:1937-1947, (2002)). In DLBCL, tumors originating from the germinal center B-cells are sensitive to chemotherapy and have a much higher chance of survival, while those from activated B cells tend to be more difficult to treat. These cell subtypes are thus dependent on the origin of the tumor cell.

Stratification of these subtypes is dependent upon the tumor's cell of origin. While in a few cases differences in subtypes can be determined by analysis of a single gene, entire arrays of combinations of genes are more determinative. Charting gene expression patterns through microarray analysis of gene expression levels would be a desirable indicator of tumor properties in other diseases such as lymphomas, acute leukemia, breast cancer, lung cancer and liver cancer. However, to adapt this genetic information for diagnostic use requires resolution of inherent significant signal-to-noise issues in present state-of-the-art technology.

Thus, there is great interest in the development of new methods for analyzing gene expression, especially where such methods provide for fast hybridization, highly specific binding of targets to complementary probes, and substantially improved signal-to-noise ratios. In addition, these methods have additional importance when assessing gene expression as it relates to cancer and disease related states (see U.S. App. 10/079,939 and U.S. App. 09/904,472 both of which are fully incorporated by reference herein).

### SUMMARY OF THE INVENTION

The present invention provided methods, apparatus, and kits for assessing gene expression in amplified mRNA isolated from circulating rare cells (see FIG. 1) which overcome the disadvantages of the prior art techniques which are described above. The present invention provides methods of isolating soluble or releasable cytoplasmic biomolecules from a single target cell or a cell population while maintaining structural cell integrity or phenotypic characteristics. Accordingly, cell(s) are either fresh or stabilized and fixed with a cross-linking agent, contacted with a pore-forming permeabilization composition, and the nucleic acids recovered. RNA from stabilized cells (PCT/US02/26867) is recovered via combinations of proteinase and nucleophiles reversal agents either for amplification and subsequent qualitative and quantitative PCR analysis or for quantitative analysis via gene specific subsets of reverse transcription (RT) primers fused with and followed by universal primer PCR amplification. Thus, one focus of the present invention prepares the cells for both cytoplasmic biomolecule analysis and phenotypic cell analysis by stabilizing and fixing cells prior to permeabilization and then releasing nucleic acids from the same stabilized cells.

The present invention is also directed to separating nuclear and/or mitochondrial DNA, RNA, proteins and other soluble components within a targeted cell by contacting a cell or cell population with a permeabilization composition and separately analyzing the released and/or unreleased fraction for one or more constituents such as the nuclear and/or mitochondrial DNA, total RNA, mRNA, soluble proteins, and other target substances (U.S. App. 60/330,669).

The present invention incorporates the analysis of both cytoplasmic biomolecules and membrane or surface biomolecules from the same cell(s) or cell population by contacting the cell(s) with a permeabilization composition and separately analyzing the cytoplasmic biomolecules and the surface biomolecules to generate functional cell profiles encompassing characteristics derived from genotypic and phenotypic cell characteristics for differentiating normal from transformed cells.

The isolation and rare cell analysis of the present invention are combined to provide the methods and reagents enabling comprehensive profiling mRNA acquired from rare cells. For example, those populations of cells defining circulating tumor cells (CTC) would be a type of rare cell found in peripheral blood and bone marrow of cancer patients. The mRNA is obtained through the cell preparation described in the present invention, but could also incorporate any protocol commonly used in the art.

After isolation and purification of mRNA from a sample containing the cells of interest, detection of extremely rare cell events with low mRNA copy numbers is achieved through gene specific RT-PCR panels with or without T7 RNA polymerase (T7RNAP) based pre-amplification procedure (U.S. App 60/369,945). Pre-amplification is completed by linear amplification of the entire mRNA library using modifications of the Eberwine aRNA method (Van Gelder et al. 1990). In a preferred embodiment, generation of an anti-sense mRNA library (aRNA) library preamplificaiton results in at least a thousand-fold increase of all messages present in the original mRNA isolated from ferrofluid enriched circulating cells. Gene specific primers are then used to amplify only the gene panel of interest. These primers are designed to amplify transcripts indicative of known rare events like circulating tumor cells. The number of target sequences can be as small as two or as large as necessary to allow correlation with some indicative characteristic of the rare event. This can occur as separate individual reactions or within a single reaction vial. Subsequent analysis yields at least a qualitative assessment of the target sequences and is achieved with methods such as, but not limited to, one of two types of multigene analysis methods we present here as gene specific primed (GSP) arrays and/or GSP sets-RT (universal PCR).

Universal PCR achieves multigene analysis from sample recovered mRNA in a single reaction tube with or without mRNA library preamplification. No preamplification allows only one panel of genes to be analyzed at one time. Preamplification adds the advantage of analyzing a single sample in up to 1000 different reactions, thus many different panels of genes can be interrogated at different times. While it will be noted that other methods are available, analysis of universal PCR cocktail panels is accomplished by array or capillary gel electrophoresis (CGE). The system allows, therefore, for both a quantitative and qualitative determination of 1 to thousands of separate mRNA types simultaneously when measured in cDNA microarray format.

Thus, the present invention includes a combination of the above mentioned isolation and profiling analysis directed to protocols and kits comprising some or all necessary reagents including a permeabilization composition, RNA recovery after cross-linking, magnetic microspheres with oligo(dT) probes covalently bound to the surface, and other gene specific magnetic microsphere-bound probes for capture and analysis of comprehensive RNA analysis using a small or large microarray, capillary gel electrophoresis (CGE), HPLC, electrophoresis and other analytical platforms.

### BRIEF DESCRIPTION OF THE DRAWINGS

FIG. 1 shows a flowchart depicting the variety of capabilities and options enabled by the inventions described in this application for multiparameter analysis on a single sample. Phenotypic and genotypic analysis is obtained on fixed or non-fixed cells.

FIG. 2 shows the reverse image (negative) of denatured total RNA analyzed by 2% agarose gel electrophoresis after SYBR Gold staining of about 1600 SKBR3 breast cancer cells that were Immuniperm-treated for various times before total RNA was isolated from the resultant supernatant via Trizol plus pellet paint co-precipitate.

FIG. 3 shows a 1% denaturing total RNA agarose gel stained with ethidium bromide comprising whole cells, Immuniperm (saponin)-permeabilized cells from the cell pellet fraction, and cells from the supernatant fraction of Immuniperm®-permeabilized SKBR3 breast cancer cells.

FIG. 4 shows a phosphor image of a Northern blot of the gel shown in FIG. 3 hybridized by a polynucleotide kinase treated ³²P-labeled oligo(dT) (25 mer) probe. The radioactive signals correspond to all the poly(A)+ mRNA transcripts of the total RNA which was derived from whole cells and the two Immuniperm® treated cell fractions from the gel shown in FIG. 3.

FIG. 5 shows a Northern blot from FIG. 4 stripped through conventional dissociation and removal of the labeled oligo(dT) probe and re-probed with a nuclear-specific precursor rRNA probe.

FIG. 6 shows the Northern blot, from FIG. 5, that was stripped and re-probed with mitochondrial-specific 12s rRNA probe.

FIG. 7 shows a cDNA array dot blot hybridization pattern comparison when the corresponding mRNA, used to generate the gel images in FIG. 3, is alpha-³²P-nucleotide labeled during first strand oligo(dT) primed cDNA synthesis. Labeled first strand cDNA was then used as the hybridization probe. Pattern comparison shows the same relative abundance of mRNA exists in all three RNA cell fractions.

FIG. 8 shows the gel image of the relative cytosolic total RNA, both quantitatively and qualitatively, obtained after separately treating multiple aliquots containing about 770 PC-3 cells each with Immuniperm®.

FIG. 9 shows the preservation, recovery and RNA integrity analysis of 90-100% of the total RNA library using CytoChex™ and other aldehyde based fixatives followed by enzyme digestion. In this experiment, mass normalized portions of 300,000 SKBR3 cell line cells which were first spiked into freshly drawn 7.5 ml peripheral blood (EDTA Vacutainer tube) in both control lanes without (phosphate buffered saline, PBS) and with three different fixatives being Cyto-Chex™, Stabilcyte™ and Transfix™. After mixing these were allowed to incubate at room temperature (20-25° C.) for 24 hours. After which the SKBR3 cells were enriched from the blood using VU-1D9 (EpCAM)-ferrofluid immunomagnetic selection. The enriched cells from each treatment were split into 3 equal aliquots and treated with proteinase K (lane labeled “Post”) or without proteinase K digestion (lanes labeled “Pre” for immediate RNA isolation, and, lane labeled “No” meaning the only difference to Post is that No proteinase K component was added and all other manipulations are equal to Post). The resultant normalized RNA isolations were separated with a 1% denaturing agarose, stained with SYBR Gold, alpha imager densitometry imaged and then Northern Blotted and finally oligo(dT) probed to show relative quality and quantity of respective total RNA and mRNA libraries recovered.

FIGS. 10A & 10B shows relative rate of Cyto-Chex™, Stabilcyte™, Transfix™, paraformaldehyde, formaldehyde, glutaraldehyde and glyoxal fixation over a 1, 2, and 4 hour time course. In this experiment, the relative-rate time course of Cyto-Chex™, Stabilcyte™, Transfix™, paraformaldehyde, formaldehyde, glutaraldehyde and glyoxal fixation were evaluated at 1, 2, and 4 hours. Samples of 7.5 ml of blood were prepared form a single donor by the same method as described in FIG. 9. The only difference is that they were selected and processed for RNA isolation at 1, 2, and 4 hour time end points. The resultant normalized RNA isolations were separated with a 1% denaturing agarose, stained with SYBR Gold, alpha imager densitometry imaged and then Northern Blotted and finally oligo(dT) probed to show relative quality and quantity of respective total RNA and mRNA libraries recovered.

FIG. 10C shows relative rate of fixation of Cyto-Chex™ vs. paraformaldehyde over a 15, 30, and 45 min time course. In this experiment, the relative-rate time course of fixation Cyto-Chex vs. paraformaldehyde were evaluated at 15, 30, and 45 min. Samples of 7.5 ml of blood were prepared from a single donor by the same method as described in FIG. 9. The only difference is that they were selected at 15, 30, and 45 min time end points. Phosphoimaging quantitation of the oligo(dT) probed blots showing relative mRNA library quality and quantity showed the relative rate of: Formaldehyde=Paraformaldehyde (˜4 fold)>Transfix™ (˜2 fold)>Stabilcyte™ (˜1.5 fold)>Cyto-Chex™ (˜1×)=glutaraldehyde and glyoxal too slow to rank.

The resultant normalized RNA isolations were separated with a 1% denaturing agarose, stained with SYBR Gold, alpha imager densitometry imaged and then Northern Blotted and finally oligo(dT) probed to show relative quality and quantity of respective total RNA and mRNA libraries recovered.

FIG. 11 shows the effects of variations on nucleophile and enzyme on the quality and quantity of RNA recovery from Cyto-Chex™ preservation supporting that when used in combined treatments for improved sequence analysis quality is likely. The resultant normalized RNA isolations were separated with a 1% denaturing agarose, stained with SYBR Gold, alpha imager densitometry imaged and then Northern Blotted and finally dT probed to show relative quality and quantity of respective total RNA and mRNA libraries recovered.

FIG. 12A shows the feasibility of diagnostic applications demonstrated by detection of specific mRNA From 10 SKBR3 cells/7.5 ml Blood in 24 hr Cyto-Chex™ stabilized blood with proteinase recovery and aRNA preamplification. Feasibility of diagnostic applications are here demonstrated by sensitive and reproducible detection of specific mRNA from triplicate 10 or 20 SKBR3 cells spiked into 7.5 ml peripheral blood. The spiked blood was stabilized immediately treated with Cyto-Chex to stabilize the cellular RNA. After incubating for one day at room temperature (20-25 C) the stabilized cells were selectively enriched using VU-1D9 (EpCam)-Ferro Fluid Immunomagnetic selection. Enrichment was followed by proteinase K digestion to liberate the RNA so that silica binding RNA isolation followed by aRNA preamplification and gene specific quantitative RT-PCR could be performed for CK19 and EpCAM.

FIGS. 12B and 12C shows CK19 and EpCAM respective Q-PCR from SKBR Cell Spike, ferrofluid selection, and CytoChex™ treatment, followed by proteinase reversal. This experiment shows the results of the quantitative RT-PCR analysis, which was normalized to original total RNA mass prior to graphing. Thus, the values shown are equivalent to the original mRNA population contained in the original RNA isolation prior to aRNA amplification.

FIGS. 12D and 12E shows CD 19 and EpCAM respective Q-PCR on aRNA derived from SKBR cells treated with cell stability reagents. These experiments show the RNA derived from the three different fixatives, which were shown in FIG. 9 Cyto-Chex™, Stabilcyte™ and Transfix™. These proteinase K recovered RNA samples were also here subjected to the same mRNA template analysis of aRNA preamplification and gene specific quantitative RT-PCR for CK19 and EpCAM as shown in FIGS. 12B and 12C respectively. These data shown here are normalized to both total RNA mass, which is equal to the aRNA mass yield in this experiment.

Thus this fixative derived relative RNA quantity and quality comparison is a measure of both aRNA (FIG. 12A) and followed by the quantitative RT-PCR shown here. Both of these comparisons are a measure of the respective fixative dependent RT template quality after the RNA was recovered using only the proteinase K method.

FIG. 13A shows RT-PCR amplification efficiency of a CK19-cRNA standard containing the 3′-most 800 base sequence of the CK19 mRNA transcript. Serial two-fold dilutions of the CK19-cRNA standard containing 200, 100, 50, 25, 12,5 copies were spiked into 2 ng of CK19 negative total RNA from white blood cells in triplicate resulting in a maximum coefficient of variation of 27%. Standard deviation bars are shown. Dilutions of cRNA to less than one copy and no template controls did not produce detectable signals.

FIG. 13B shows the relative RT-PCR gene expression levels after agarose gel electrophoresis.

CK19 cRNA was spiked into total RNA from white blood cells at levels of 25 copies, 250 copies, 2,500 copies and 25,000 copies in panel 1, panel 2, panel 3, and panel 4, respectively.

FIG. 13C compares the relative representation in the same mRNA library of unamplified and T7 promoter-based amplified mRNA transcripts. Relative abundance was assessed by examining 8 different mRNA transcripts (PSA, PSM, MGB1, MGB2, CK8, CK19, PIP, EpCam) using the RT-PCR kinetic curve method.

FIG. 14A shows scatter plot bar graphs of a survey of genes indicating the presence of circulating epithelial cells. Human blood, immunomagnetically enriched for cells expressing the EpCAM antigen on their cell surface, the samples were first aRNA preamplified and then 25 ng were reverse transcribed. Aliquots were then analyzed by agarose gel electrophoresis after RT-PCR on a select group of genes. 13 healthy donors (7 male, 6 female) are designated as the N column for each gene measured and 9 serially sampled HRPC patients containing circulating tumor cells (CTC) were determined by flowcytometry and are designated as the P column for each gene measured. Horizontal lines in each column indicate threshold values above which true positives were counted.

FIG. 14B depicts a survey of genes indicating prostate tumor organ of origin status via the same methods as described in 14A.

FIG. 14C depicts a survey of genes indicating the presence of therapeutic target status via the same methods as described in 14A.

FIGS. 15A, 15B, and 15C show individual HRPC patient longitudinal monitoring of CTC and RT-PCR multigene analysis before, during, and after new line chemotherapy. The x-axis shows sampling time in weeks, the left y-axis shows the CTC level with the solid circle symbol. The right y-axis shows the relative mRNA expression levels with corresponding symbols of open-square for Androgen Receptor (AR), open circle for Hepsin (HPN) and open triangle for multidrug resistance (MDR1). Relative mRNA levels are illustrated here during treatment courses of Lupron alone as shown FIG. 15A, and 2 patients being treated with Lupron combined with administration of doses of Taxotere and Estramustine chemotherapy symbolized by the vertical arrows on the x-axis (Tx/Ex) in FIG. 15B and FIG. 15C. Bars on top indicate long term hormonal ablation treatment was on going.

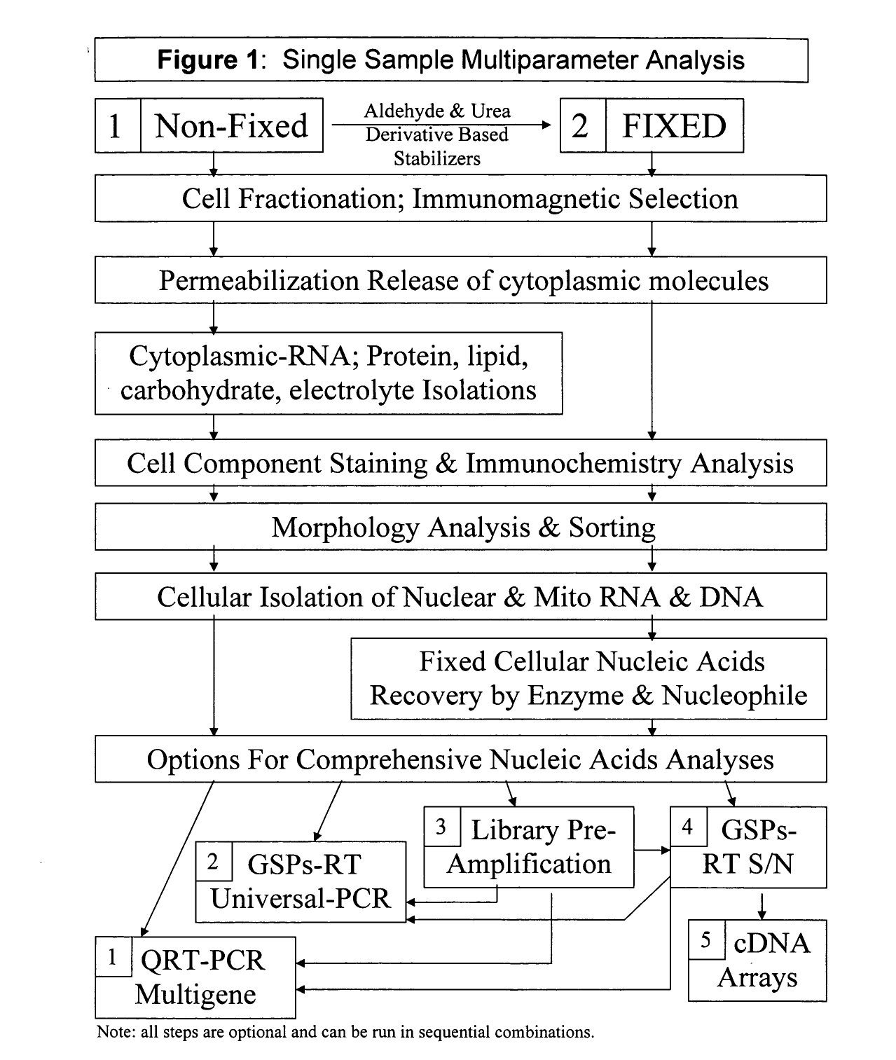

### DETAILED DESCRIPTION OF THE PREFERRED EMBODIMENTS

As has been indicated in the foregoing discussion, a more comprehensive and practical form of cancer diagnosis must also include analysis of intra- and extra-cellular membrane antigens as well as analysis of cellular RNA content and DNA content in the same cell or cell population, which up to now have been mutually exclusive processes (U.S. Pat. No. 6,365,362). This exclusivity was due to the basic incompatibility of pre-analytical cell preparation methods for analyzing structural intracellular antigens, having the major objective to maintain cell integrity, with methods of isolating cytoplasmic biomolecules. Alternatively, pre-analytical cell preparation could also be limited to soluble cytoplasmic RNA, total cellular RNA, total cellular DNA, and/or proteins, having the major objective to homogenize cells in order to release soluble intracellular components (U.S. Pat. No. 6,329,179). In particular, traditional phenotypic characterizations required fixation of cell structures achieved through exposure of cells to a cross-linking agent, such as paraformaldehyde, formaldehyde, glutaraldehyde, etc. These harsh cell fixation conditions simultaneously cause undesirable covalent crosslinking and/or fragmentation of all the isolatable RNA species. Similar intracellular DNA-protein cross-links have recently been reported (Quievryn and Zhitkovick, Loss of DNA-Protein Crosslinks from Formaldehyde-Exposed Cells Occurs Through Spontaneous Hydrolysis and an Active Repair Process Linked to Proteosome Function, Carcinogenesis, 21:1573-1580 (2000). So-called non-formaldehyde or non-paraformaldehyde fixatives (e.g., Cyto-Chex™Streck Labs, Omaha, Nebr.) are cell-stabilizing additives containing formaldehyde-urea derivative donor compounds. It is used as a preservative for circulating tumor cells in blood during shipment or storage as disclosed in a co-pending application (PCT/US02/26867 incorporated by reference herein). However, the studies conducted by the present inventors have shown that even Cyto-Chex™, which contains only trace levels of free formaldehyde, apparently slowly releases formaldehyde that can cross-link intracellular RNA to intracellular proteins. Such cross-links were fully reversed by the methods of this invention to allow comprehensive RNA analysis. Cellular RNA and DNA analysis are therefore conventionally prepared on unfixed fresh cells or cells that are preserved with reagents that do not cross-link or of which the cross-linking can be reversed during the mRNA release from the cells. RNAlater™ (Ambion) is commercially available RNA stabilization solution, which stabilizes RNA but does not allow immunomagnetic, immunochemistry or image analysis on the same sample and is not effective for blood. PreAnalytiX offers a blood RNA stabilizer but is nothing more than the chaotropic agent guanidine isothiocyanate solution (GITC) solution in a Vacutainer™ tube enabling nothing more than traditional homogenization based solely on total RNA isolation.

In general, mRNA recovered from fixed cells is not quantitative and is severely degraded or fragmented reducing the size of intact RNA with an average size of approximately 1750 bases as much as ten-fold to a highly variable average size of approximately 200 bases, and contains many complex chemical modifications, which are not well understood. However, the net effect of fixative derived RNA is severely compromised mRNA analysis (Current Protocols in Molecular Biology, Wiley, (2002)). Tedious non-quantitative mRNA salvage techniques combined with reverse transcriptase polymerase chain reaction (RT-PCR) analysis designed for amplicons of less than 100 base pairs in length show limited value, albeit in a qualitative not quantitative manner (U.S. Pat. No. 5,346,994). Further, this limited RNA analysis of fixed cells must follow phenotypic analysis. Thus, the two processes cannot be run sequentially on the same cell sample, because traditional RNA isolation techniques require complete cell lysis or homogenization, destroying cell structure and further complicating analysis by intermingling the cellular DNA and RNA populations (Maniatis et al., Molecular Cloning-A Laboratory Manual, 2^{nd} ed., Cold Spring Harbor Press (1989)). Previous reports have shown a need for improving methods of RNA recovery in tissue (Godfrey, et al., Quantitative mRNA Expression Analysis from Formalin-Fixed, Paraffin-Embedded Tissues Using 5′ Nuclease Quantitative Reverse Transcription-Polymerase Chain Reaction, J. of Molecular Diagnostics, 2:84-91 (2000)).

Applications of formaldehyde and urea based fixatives that stabilize recoverable quantitative, high quality full-length intact total and mRNA libraries from blood and thus enabling comprehensive analysis are the basis of one aspect of this invention.

Quite unexpectedly, saponin, used as a permeabilizing agent, was found to be a highly selective and efficient releasing agent for intracellular cytoplasmic RNA and other biomolecules, thereby obviating the need for cell lysis or homogenization. This novel use of saponin as the RNA releasing agent of choice is a particularly advantageous component of the present invention. Surfactants such as saponin have traditionally been used to examine the expression of intracellular antigens by permeabilization of the cell membrane allowing for incorporation of staining reagents while maintaining cell integrity. For instance, analysis of chromosomes or genes by fluorescence in situ hybridization (FISH), or in staining of intracellular constituents, such as DNA in nuclei, with the nuclear stain, DAPI, or in immunostaining of cytokeratins with specific labeled antibodies play a critical role. Release of cytoplasmic intracellular proteins RNA or DNA generally is done by solubilization or complete lysis of the cells with stronger surfactants, such as Triton X-100. Saponin, however, has heretofore not been used to study both expression of soluble intracellular antigens including RNA and phenotyping of individual cells or cell populations in the same specimen. Accordingly, methods allowing sequential phenotypic analysis as well as analysis of intact RNA and soluble proteins in the cytoplasm of the same cell specimen are highly desired and are the subject of this invention.

Accordingly, the present invention provides advantageous methods, apparatus, and kits for the rapid and efficient RNA profiling of all cells and especially targeted cells found in biological samples. The present invention provides methods for allowing separate analysis of both phenotype and genotype. Phenotype is interrogated and profiled via antibody antigen protein and mass spectrometry profiling methods and comprehensive analysis of intact cytoplasmic RNA from the same cell or cell population. Genotyping of the sample genomic and mitochondrial DNA can be separately profiled by any means available to those skilled in the art.

Similar to the amplification of the mRNA library, the respective genomic and mitochondrial libraries can be preamplified enabling numerous assays to be performed without loss of clinical sensitivity due to Multiple Displacement Amplification (MDA) technology enables the first effective whole genome amplification method. MDA is a rapid, reliable method of generating unlimited DNA from a few cells.

The invention described herein may be used effectively to isolate and characterize cell phenotype, such as cell surface antigens, intra-cytoplasmic antigens and any type of RNA, and genotype. Both phenotypic and genotypic analysis can be performed sequentially on the exact same sample. For example after cell surface analysis and RNA harvesting, the remaining intact nuclei and mitochondria can be analyzed downstream by all standard RNA (mt RNA, hRNA), DNA and protein based analysis techniques such as S1 nuclease, ribonuclease protection, RT-PCR, SAGE, DD-RT-PCR, microarray cDNA hybridization, ISH, FISH, SNP, all RNA and all genomic-based PCR techniques and any protein analysis systems.

One of the many applications of this type of cell analysis is in cancer diagnostics. Many clinicians believe that cancer is an organ specific disease when confined to its early stages. The disease becomes systemic by the time it is first detected using methods currently available. Accordingly, evidence to suggest the presence of tumor cells in the circulation would provide a first line detection mechanism that could either replace, or function in conjunction with other tests such as mammography or measurements of prostate specific antigen. By analyzing cellular phenotype (protein and RNA) and genotype through specific markers for these cells, the organ origin of such cells may readily be determined, e.g., breast, prostate, colon, lung, ovarian or other non-hematopoietic cancers. Thus in situations where protein, RNA, and genome can be analyzed, especially where no clinical signs of a tumor are available, it will be possible to identify the presence of a specific tumor as well as the organ of origin. As these profiles define cell function, they also indicate what the most appropriate therapy type and course should be when used in cancer cell detection. Further in monitoring cases where there is no detectable evidence of circulating tumor cells as with post operative surgery or other successful therapies, it may be possible to determine from a further clinical study whether further treatment is necessary.

In order to provide for a more comprehensive and early diagnosis, one embodiment of the invention includes the methods for isolating cytoplasmic biomolecules from a cell or population of cells, contacting the cell or cells with a permeabilization compound, and isolating the cytoplasmic biomolecule of interest from the cell while maintaining cell integrity for subsequent phenotypic and morphological analysis.

The targeted rare event in this invention refers to the expression of any biomaterial indicative, at least in part, to a known rare event. Accordingly, hormones, proteins, peptides, lectins, oligonucleotides, drugs, chemical substances, nucleic acid molecules (such as RNA and/or DNA) and bioparticles such as cells, apoptotic bodies, cell debris, nuclei, mitochondria, viruses, bacteria, and the like would be included in the embodiment of this invention.

The fluid sample includes, without limitation, cell-containing bodily fluids, peripheral blood, bone marrow, urine, saliva, sputum, semen, tissue homogenates, nipple aspirates, and any other source of rare cells that is obtainable from a human subject.

“Cytoplasmic biomolecules” includes cellular target molecules of interest such as, but not limited to, protein, polypeptides, glycoprotein, oligosaccharide, lipids, electrolytes, RNA, DNA and the like, that is located in the cytoplasmic compartment of a cell. Upon contacting a cell with a permeabilization compound and subsequent cell separation, the cytoplasmic biomolecules are present in the supernatant for downstream analysis. All soluble cytoplasmic biomolecules, for example, the entire cytoplasmic RNA library or target components capable of traversing the membrane pores can be isolated and analyzed. In a preferred embodiment, the focus is on the analysis of transcribed mRNA and translated proteins, for example in CTC, as indicators of oncogenic transformations of interest in the management of cancer diagnosis and therapy.

“Membrane biomolecules” includes any extracellular, intra-membrane, or intracellular domain molecule of interest that is associated with or imbedded in the cell membranes including, but not limited to, the outer cell membrane, nuclear membrane, mitochondrial and other cellular organelle membranes. Upon permeabilization with a permeabilization compound of this invention, the targeted membrane biomolecules are normally not solubilized or removed from the membrane, i.e. the membrane biomolecules remain associated with the permeabilized cell.

Membrane biomolecules include, but are not limited to, proteins, glycoproteins, lipids, carbohydrates, nucleic acids and combinations thereof, that are associated with the cellular membrane, including those exposed on the external or extracellular surface of the outer membrane as well as those that are present on the internal surface of the outer membrane, and those proteins associated with the nuclear, mitochondrial and all other intracellular organelle membranes. Membrane biomolecules also include cytoskeletal proteins.

“Genotype” or “genotyping” refers to the process of identifying intracellular genetic materials, such as DNA, that store internally coded inheritable instructions for constructing and controlling all aspects of cell life and death. “Phenotype” or “phenotyping” is defined as classifying a cell on the basis of observable outward structural elements and the production thereof (i.e. including the intermediate RNA). These include topology, morphology and other surface characteristics, all of which result from internally coded genotypic information which are incorporated into the methods of the present invention. In contrast, cell structure and integrity are not maintained during conventional RNA isolation techniques involving complete lysis of, at least, all cell structures except for nuclei and mitochondria in the presence of NP-40, usually by disintegration of all cell structures during chaotropic salt treatment and/or mechanical cellular homogenization.

Morphologic or morphology in reference to cell structure is used as customarily defined, pertaining to cell and nuclear topology and surface characteristics including intracellular or surface markers or epitopes permitting staining with histochemical reagents or interaction with detectably labeled binding partners such as antibodies. In addition morphology shall include the entire field of “morphometry” defined as: quantitative measure of chromatin distribution within the nucleus.

The terms genomic and proteomic are used as conventionally defined. “Functional” is herein used as an adjective for an empirically detectable biological characteristic or property of a cell such as “functional cellomic” which more broadly encompasses both genomic and proteomic as well as other target categories including, but not limited to, “glyconomic” for carbohydrates and “lipidomic” for cellular lipids. The resultant cell characteristics provide profiles permitting differentiation of normal from transformed cells.

“Contacting” means bringing together, either directly or indirectly, a compound or reagent into physical proximity of a cell. The cell and/or compounds can be present in any number of buffers, salts, solutions, etc. Contacting includes, for example, placing the reagent solution into a tube, microtiter plate, microarray, cell culture flask, or the like, for containing the cell(s). The microtiter plate and microarray formats further permit multiplexed assays for simultaneously analyzing a multiplicity of cellular target compounds or components including, but not limited to, nucleic acids and proteins.

“Permeabilization compound, reagent, or composition” means any reagent that forms small pores in the cell membranes, comprising the lipid-cholesterol bilayer, while maintaining sufficient membrane, cytoplasmic and nuclear structure such that subsequent phenotypic analysis can be carried out on the permeabilized cell(s). For example, saponin is a known “pore-forming” compound that complexes with cell membrane components thereby forming numerous trans-membrane pores of about 8 nm size in the cell wall or membrane, thus allowing outward diffusion of small soluble cytosolic constituents, such as enzymes, proteins, glycoproteins, globulins, electrolytes, and the like, and internal equilibration with extracellular reagent components, such as electrolytes.

“Immunomagnetic beads” are magnetically labeled nanoparticles or microparticles also having covalently attached binding reagents (e.g. antibodies) with substantially selective affinity for surface markers or epitopes on cells, thereby achieving selective capture of magnetically labeled cells when exposed to a magnetic field such as generated in high gradient magnetic separation system (HGMS). Other terms used herein for methodologies, reagents and instruments are as conventionally defined and known to persons skilled in the art.

Preferred gene expression targets (mRNA and protein) for identifying tissue of origin, diagnosis, prognosis, therapy target characterization and monitoring include but are not limited to cells derived from cancers of the breast, prostate, lung, colon, ovary, kidney, bladder, and the like for the purpose of detection and monitoring of sensitive or resistant genes expressing markers such as mammoglobin 1 (MGB 1), mammoglobin 2 (MGB2), Prolactin inducible protein (PIP), carcinoembryonic antigen (CEA), prostate specific antigen (PSA), prostate specific membrane antigen (PSMA), glandular kallikrein 2 (hK2), androgen receptor (AR), prostasin, Hespin (HPN), DD3, Her-2/Neu, BCL2, epidermal growth factor receptor (EGFR), tyrosine kinase-type receptor (HER2), thymidylate synthetase (TS), vascular endothelial growth factor VEGF, pancreatic mucin (Muc1), guanylyl cyclase c (GC-C), phosphatidylinositol 3 kinase (PIK3CG), protein kinase B gamma (AKT), excision repair protein (ERCC1), alpha-1 globin (F6), macrophage inhibitory cytokin-1 (G6), dihydropyrimidine dehydrogenase (DPYD), insulin growth factor receptor (IGF2) estrogen receptors alpha and beta (ER), progesterone receptor (PR), aromatase (cyp19), Telomerase (TERT), general epithelial tissue specific genes, cytokeratin 19 (CK19), cytokeratin 5 (CK5), cytokeratin 8 (CK8), cytokeratin 10 (CK10), cytokeratin 20 (CK20), epithelial cell adhesion molecule (EpCAM), mucins including mucin 1 (MUC1), topoisomerases, urokinase plasminogen activator (uPA), urokinase plasminogen activator receptor (uPAR), matrix metalloproteinases (MMP), general white blood cell specific mRNA, alpha-1-globin, CD16, CD45, and CD31, and the like. This list is intended to illustrate the general diversity of arrays of mRNA-specific genes that could be assembled to differentiate cells from diverse origins, types and diseases, and is not intended to be comprehensive.

Stabilization, Release, and Recovery

Using the method of a previously disclosed invention, commonly assigned herewith, U.S. Pat. No. 6,365,362 and U.S. application Ser. No. 10/079,939 (both of which are incorporated by reference herein), circulating epithelial cells can be enriched relative to leukocytes to the extent of at least 2,500 fold to around 10,000 fold. Immunomagnetic selection of circulating epithelial cells in blood is followed by a nucleotide analysis embodied in this invention. The enrichment is only one example of many methods known in the art for selecting specific populations of cells to be used in the embodiment of this invention.

A method of releasing intact cytoplasmic total RNA and mRNA from these cells, thereby isolating and purifying them, was unexpectedly and surprisingly discovered during conventional permeabilization of cells with saponin prior to staining and immunostaining, thereby enabling sequential or parallel analysis of both cytoplasmic RNA and intracellular antigen phenotyping and DNA genotyping on the exact same cell, population of cells, or specimen.

Permeabilization can be accomplished under this criteria using 1 of 3 types of general surfactants or detergents: pore forming reagents, like saponin, or saponin fractions such as QS-21, escins, digitionin, cardenolides, etc. All of these agents increase membrane porosity and release small soluble intracellular components. Another group of agents are surfactants. These agents have a relatively high hydrophilic-lipophilic balance to permeate the membrane without lysis. Other, more lytic surfactants with a lower hydrophilic-lipophilic balance, would release RNA, but tend to solubize the membrane. These include, but not limited to, polyoxyethylene sorbitans (commercially known as Tween 20, 40, or 80), nonylphenoxy polyethoxy ethanol (NP-40), and the like, t-octyl phenoxy ethoxy ethanol, or SDS.

Subsequent analysis of cytoplasmic RNA (and other RNA such as mtRNA and hnRNA), cell surface as well as soluble intracellular antigens, cell organelles such as mitochondria and the remaining indexed nuclei can then be analyzed downstream by all standard RNA, DNA, and protein based analysis techniques. These include all types of cDNA, RNA and protein microarrays for profile analyses, mass spectrometry, fluorescent in situ hybridization (FISH), single nucleotide polymorphism (SNP), all genomic-based amplification techniques such as PCR and the like, microsatellite analysis, restriction fragment length polymorphism (RFLP, ALFP), SAGE, DD-RT-PCR, and the like.

Such analyses can be conducted on as few as 1-10 RNA molecules for each and any RNA sequence type, but preferably on tens of thousands up to millions copies of targets to enable detection of subtle alterations in cellular translation or transcription profiles as indicators of disease states in a clinical setting. Other functional cell profiles of releasable and non-releasable cellular components, such as proteins, glycoproteins, lipoproteins, oligoglycosides and the like, can similarly be generated by analyzing the two fractions by conventional microarray, HPLC, electrophoretic methods including the high-resolution 2D electrophoresis method, or antibody array profiling.

Permeabilization compounds of this invention include, but are not limited to, saponins, a class of natural products constructed of cholesterol-like aglycones or genins (triterpenes or steroids not bearing any carbohydrate moieties) linked to fatty acids and one or more carbohydrates, which disperse readily in water to form globular micelles, the active species in pore formation. These and the other above named suitable pore formers, polyoxyethylene sorbitans (commercially known as Tween 20, 40, or 80), nonylphenoxy polyethoxy ethanol (NP-40), and t-octyl phenoxy ethoxy ethanol, have a high HLB (hydrophilic-lipophilic balance) numbers which must be used at sufficiently low concentrations to minimize undesirable solubilization of cellular components and membrane lysis. The concentration range of the permeabilization compound is about 0.01-0.5% (w/v) when using saponin containing about 10% sapogenins. A preferred permeabilization compound is saponin (Sigma Catalog Number S-7900). Saponins from other sources and of higher purities may also be used, for example, saponin of about 20-25% purity as sapogenin (Sigma S-4521) and a highly purified saponin, QS-21, of about 99% purity available from Aquila Biopharmaceuticals, Framingham, Mass. Other usable compound are alpha-escin and beta-escin (Sigma E-1378), both derived from horse chestnuts. The permeabilization compound may be present in a composition, such as phosphate buffered solution, that also comprises antimicrobial agents such as, for example, sodium azide, Proclin 300 (Rohm&Haas, Philadelphia, Pa.), and the like. Another preferred permeabilizing agent is Immuniperm™, which by itself releases about 50% of the cytoplasmic RNA (85% of all RNA in the cell) with no affect on the nuclear or mitochondrial nucleotide pools. The remaining 50% of the total cellular RNA and all DNA in fixed cells can be released with a releasing cocktail comprising SDS, protease, and a formaldehyde scavenging agent, which composition constitutes one embodiment of this invention. While the exact mode of action of the individual cocktail components is unknown, it is speculated that the SDS serves to solubilize intracellular RNA and DNA crosslinked to structural intracellular proteins thereby enabling more efficient proteolysis and release of formaldehyde cross-linked nucleic acids. The novel formaldehyde scavenging reagents, exemplified by, but not limited to, hydroxylamine, carboxymethoxylamine, hydrazine, acethydrazide and other hydrazides, or hydrazine derivatives, and amines such as tris, were found to increase the amount and “quality” of the released nucleic acids, where quality is measured by increased amplification rates and yields. The two fractions released with Immuniperm and the releasing cocktail can be individually analyzed or pooled prior to analysis.

Accordingly, any surfactant or protease (or combination thereof) with or without added formaldehyde scavenger, capable of releasing cellular nucleotide stores and maintain a suitable morphology for concurrent analysis, would be included within the scope of the present invention.

Unlike current cellular fixation and RNA salvage protocols that tend to significantly fragment cellular RNA, the present invention enables extraction and isolation of greater than 90% of the intact cytoplasmic total RNA and mRNA from cells treated with a permeabilization agent, such as saponin, that permeabilizes the cell membrane while maintaining cell integrity. The mRNA isolation is also compatible with immunomagnetic cell enrichment and immunofluorescent cell labeling procedures. Comprehensive RNA expression profile analysis of cells identified and characterized by cell analysis platforms such as RNA polymerase promoters based linear amplification methods employing T7, SP6, or T3 promoters, flowcytometry, microarrays and in Cell Spotter® or CellTracks systems (both manufactured by Immunicon Corp, PA) can be used to directly validate, complement and expand the expression profiles and enhance the information obtained therefrom.

While not limited to a particular fixative, permeabilized cells are treated with a cross-linking agent to maintain morphological, antigen and nucleotide integrity as stated above. Cyto-Chex™, StabilCyte™ and TRANSfix™ are examples of three commercially available stabilizers that have shown utility in stabilizing blood cells in blood specimens for extended time periods. These stabilizers are optimized to maintain cell size (mainly by minimizing shrinking) and to preserve antigens on cell surfaces, primarily as determined by flowcytometry. The intended applications generally involve direct analyses and do not require extensive manipulation of the sample or enrichment of particular cell populations. In contrast, the circulating tumor cells, or other rare target cells, isolated and detected in this invention, comprise and are defined as pathological abnormal or rare cells present at very low frequencies, thus requiring substantial enrichment prior to detection.

Cyto-Chex™ stabilizer can be used as a cell stabilizer and, as proven in application of the present invention, an aldehyde releasing fixative of intracellular RNA resulting in the formation of macromolecular complexes with intracellular proteins. We unexpectedly found that fixation, preferably with a formaldehyde donor such as Cytochex™, was essential for retaining and protecting RNA during subsequent sample processing, and that total or optimal release of fully functional RNA required saponin in combination with the above-cited release cocktail.

The ideal “stabilizer” or “preservative” (herein used interchangeably) is defined as a composition capable of rapidly preserving target cells of interest present in a biological specimen, while minimizing the formation of interfering aggregates and/or cellular debris in the biological specimen, which in any way could impede the isolation, detection, and enumeration of targets cells, and their differentiation from non-target cells. In other words, when combined with an anti-coagulating agent, a stabilizing agent should not counteract the anti-coagulating agent's performance. Conversely, the anti-coagulating agent should not interfere with the performance of the stabilizing agent. Additionally, the disclosed stabilizers also serve a third function of fixing, and thereby stabilizing, permeabilized cells, wherein the expressions “permeabilized” or “permeabilization” and “fixing”, “fixed” or “fixation” are used as conventionally defined in cell biology. The description of stabilizing agents herein implies using these agents at appropriate concentrations or amounts, which would be readily apparent to one skilled in cell biology, where the concentration or amount is effective to stabilize the target cells without causing damage. One using the compositions, methods, and apparatus of this invention for the purpose of preserving rare cells would obviously not use them in ways to damage or destroy these same rare cells, and would therefore inherently select appropriate concentrations or amounts. For example, the formaldehyde donor imidazolidinyl urea has been found to be effective at a preferred concentration of 0.1-10%, more preferably at 0.5-5% and most preferably at about 1-3% of the volume of said specimen. An additional agent, such as polyethylene glycol has also been found to be effective in stabilizing cells, when added at a preferred concentration of about 0.1%-5%. The use of such agents is described in PCT/US02/26867, and is incorporated by reference herein.

A surprising aspect of the present invention is that intracellular RNA as part of the macromolecular complex can be recovered amplifiable and in nearly quantitative yields from cells previously treated with a cell stabilizer and fixative. Full release of cross-linked RNA requires saponin in combination with enzymatic digestion in the presence of a lytic detergent and a formaldehyde scavenger. For example, proteinase K, V8 proteinase, pronase digestion of Cyto-Chex™ treated cells results in complete recovery or full-length comprehensively analyzable RNA. The presence of a formaldehyde scavenger as disclosed in the present invention was found to further improve RNA recoveries.

In the embodiments of the present invention, target cells such as circulating cancer cells or fetal cells can be assayed by efficiently isolating them from other non-target cells, purifying their nucleic acids, and then amplifying the target(s) of interest for microarray analysis.

Thus, isolation of cytoplasmic biomolecules is achieved by first separating the permeabilized cell from the permeabilization compound through centrifugation or immunomagnetic bead enrichment. The cytoplasmic biomolecule mixture is then present in the supernatant. Isolation of cytoplasmic biomolecules can be achieved by capture with magnetic beads. For example 1f the cytoplasmic biomolecules are mRNA, oligo(dT) affixed to magnetic beads or nonmagnetic supports can be used to capture and thereby separate the mRNA from the cells with or without centrifugation. If the cytoplasmic biomolecules are proteins, antibodies that are able to bind to the particular protein can be used, wherein the antibodies can be affixed to magnetic beads or nonmagnetic supports. Other isolation techniques are well known to the skilled artisan such as standard protein and RNA chemical extractions, electrophoresis, chromatography, immunoseparations and affinity techniques. Immunomagnetic enrichment reagents and devices for separating cells and biomolecules are available from several manufactures including but not limited to Immunicon Corp. (Huntingdon Valley, Pa.), Dynal (New Hyde Park, N.Y.) and Miltenyi Biotec Inc. (Auburn, Calif.). The cells can be prokaryotic, such as bacterial cells, or eukaryotic, such as mammalian cells, and are most preferable of human origin. In the preferred embodiments, the cells are carcinoma or tumor cells. Carcinomas of preferred interest include, but are not limited to, those derived from breast, prostate, lung, colon, and ovarian tissues, and the like, as found in tissue sections or in body fluids, for example, as circulating tumor cells in blood and bone marrow.

Methods are disclosed for preparing a cell for cytoplasmic and or whole cell biomolecule analysis and membrane biomolecule analysis sequentially on the exact same sample, collectively defined as either functional genomics or functional proteomics for analyses of nucleic acids or proteins, respectively. As stated above, such analyses have not heretofore been possible on the same cell(s) prior to the methods of this invention. The cells are contacted with a permeabilization compound to release cytoplasmic biomolecules, as described above, without altering structural biomolecules and membrane biomolecules.

Thus as disclosed herein in the present invention, the methods of analyzing a cytoplasmic biomolecule from a cell sample and analyzing a membrane biomolecule from the same cell sample are provided after the cells are contacted with a permeabilization compound, stabilized, and a cytoplasmic biomolecule recovered as described above. A cytoplasmic biomolecule can be isolated and analyzed concurrently or consecutively with an associated biomolecule.

This invention also provides reagents and kits for isolating cytosolic or whole cellular RNA, in particular, mRNA. The kits may include a permeabilization compound and RNA extraction reagents or hybridization probes for RNA isolation and detection, such as for example, oligo(dT) or gene-specific sequences or random (degenerate) oligonucleotides of various lengths. The kits can also include antibodies that bind to proteins associated with cells, such as antibodies that bind to membrane biomolecules. The antibodies and probes can be enzymatically labeled, fluorescently labeled, or radiolabeled to allow detection. The antibodies and probes can also be attached to, for example, magnetic beads or the like, to facilitate separation.

Analysis

Cytoplasmic biomolecule analysis includes any type of analysis or assay that involves a biomolecule isolated from the cytoplasm of a cell. Cytoplasmic biomolecule analysis further includes, but is not limited to, functional genomic expression profiling including, but not limited to, mRNA profiling, protein expression profiling, reverse transcriptase polymerase chain reaction, Northern blotting, Western blotting, nucleotide or amino acid sequence analysis, serial analysis of gene expression SAGE, competitive genomic hybridization (CGH), electrophoresis, 2-D electrophoresis, mass spectrometry by MALDI or SELDI, gas chromatography, liquid chromatography, nuclear magnetic resonance, infrared, atomic adsorption, and the like. Sequence analysis, at the nucleotide or amino acid level, can indicate and identify the presence of a mutation in a protein, DNA/cDNA, or mRNA sequence. For example, an original gene or protein profile analysis may indicate the presence of an oncogene in a transformed or tumor cell. Subsequent analysis after appropriate cancer therapy may show lower tumor burdens during remission or indicate regression as a result of further mutations of the oncogene and emergence of drug-resistant or more aggressive tumor cells.

Membrane biomolecule analysis includes any type of analysis or assay that involves a biomolecule bound to or associated with a cellular membrane within a cell, i.e. extra-cellular and intracellular biomolecules or markers. Appropriate analytical methods include, but are not limited to, flowcytometry, enzyme-linked immunosorbant assay, morphological staining, cell sorting, and the like. Permeabilized cells can be sorted by, for example, fluorescence activated cell sorting (FACS) techniques based upon the expression of a particular detectable protein. Cell sorting techniques are well known to the skilled artisan and have been used to simply count detectably labeled cells, for example, in cancer diagnosis. Permeabilized cells can also be classified on the basis of expression of a particular protein, e.g. CD4 or CD8 cells. Membrane biomolecule analysis can also be done on downstream membrane fractions followed by analysis, including, but not limited to protein expression profiling. Western blotting, amino acid sequence analysis, mass spectrometry, gas chromatography, liquid chromatography, nuclear magnetic resonance, infrared, atomic adsorption, surface plasma resonance (SPR) and any other technique suitable for analysis of membrane components.

Functional genomic analyses or assays can be performed on the genetic material that is retained within a permeabilized cell. For example, genomic DNA, nuclear (hnRNA), mitochondrial (mtRNA) and any other RNA or DNA harbored by an organelle that remains bound or fixed within the cell upon permeabilization of a cell can be assessed. Thus, the types of analyses described above for cytoplasmic biomolecules can be performed for genomic DNA, hnRNA, and mtRNA using methods or assays including, but not limited to, in situ hybridization, polymerase chain reaction, differential display PCR, arbitrarily primed PCR, microsatellite analysis, single nucleotide polymorphisms (SNP), competitive genomic hybridization (CGH), restriction fragment length polymorphism analysis, nuclear and mitochondrial transcript run-on assays, and in vitro protein translation assays. To obtain genomic DNA, nuclear hnRNA, and mtRNA, however, the permeabilized cells must either be exposed to the releasing cocktail of the present invention, completely lysed, or further fractionated by conventional means well known to the skilled artisan. For stabilized cells, combinations of proteinase and nucleophiles can be used to reverse and remove macromolecular complexes containing the nucleic acids of interest, liberating RNA and DNA nucleic acid components. Furthermore, cell organelles retained upon permeabilization can be subsequently further fractionated and isolated for metabolic functional assays of, for instance, mitochondria and the like.

Accordingly, another embodiment of the present invention provides methods of separating nuclear or mitochondrial genetic material from cytosolic RNA. Cells containing the nuclear or mitochondrial genetic material and cytosolic RNA are contacted with a permeabilization compound, as described above. Nuclear or mitochondrial genetic material can be isolated by, for example, subsequent appropriate sub-cellular fractionation and complete cell/organelle lysis of the fractionated cellular material. The resultant organelle specific components (DNA, RNA, proteins, lipids, carbohydrates, etc.) can be extracted or isolated from the homogenate and analyzed. Separation can also be accomplished using organelle-specific immunomagnetic beads, as described above.

Several important practical automation advantages accrue from the present invention. For example after the permeabilization solution has been removed form the cells, the mRNA can be captured with oligo(dT)-magnetic beads that are ideally suited for automated downstream manipulation and comprehensive analysis similar to microarrays. In addition only minor changes are required in the current mRNA analysis protocols to generate both protein and mRNA profiles thus reducing the time and reagent requirements. Further, the corresponding intact cellular genomic DNA in the nuclei and mitochondria is still contained and accessible in the permeabilized cells and can be analyzed downstream by conventional methods for DNA, RNA and protein such as FISH, SNP, SAGE, DD-PCR, PCR, RFLP, RT-PCR, CGH, cDNA microarrays, mass spectrometry and protein arrays, etc. Simultaneous multicomponent analysis strategies of DNA, RNA, protein, lipid, carbohydrate, and (precursors, metabolites, and co-factors thereof), for example, on large microarrays can thus be broadly applied to any eukaryotic cell, tissue sample or body fluid. This type of cell expression profiling by means of multicellular component or combined with multiplexed (e.g. microarray) analyses is a cutting edge objective in technologies ranging from high-throughput screening of drug candidates to disease diagnosis and management.

This invention also provides reagents and kits for isolating cytosolic or whole cellular RNA, in particular, mRNA. The kits may include a permeabilization compound and RNA extraction reagents or hybridization probes for RNA isolation and detection, such as for example, oligo dT or gene-specific sequences or random (degenerate) oligonucleotides of various lengths. The kits can also include antibodies that bind to proteins associated with cells, such as antibodies that bind to membrane biomolecules. The antibodies and probes can be enzymatically labeled, fluorescently labeled, or radiolabeled to allow detection. The antibodies and probes can also be attached to, for example, magnetic beads or the like, to facilitate separation.

The intact library is then interrogated for the presence of any messages involved in identifying the presence of epithelial cells and/or confirming the presence of the tissue of origin of those epithelial cells. To this end, all of the mRNA present in the sample must be analyzed for each particular gene of interest, each with the same sensitivity/selectivity as the other and with the ability to look at all the mRNA of interest at one time.

Under preceding criteria, global gene expression analysis by microarray would be insensitive to rare events. In particular, the signal-to-noise ratio in the sample would be impracticably low because of such problems as the white blood cell immunomagnetic carryover contamination in any given enriched sample. For example in a fluid sample enriched for a particular target population of cells by immunomagnetic selection, there potentially could be approximately 10,000 white blood cells carried over with a target population of 1 to 10 cells. The target cell(s) is expressing the rare event of interest, and would be masked by the nucleotides found in the white blood cells. The excessive white blood cell derived background RNA noise coupled with the extremely rare copy level of the target mRNA results in a potential signal that may not be detected.

To circumvent the problem, total RNA (or purified mRNA) is pre-amplified by employing either a SP6, T3, or T7 RNA polymerase promoter-based in vitro linear pre-amplification method. A typical example is T7 RNA polymerase (T7RNAP), promoter (T7RNAPP) and enzyme amplification system, but any equivalent system can be substituted by systems obvious to those skilled in the art. The linear pre-amplification of all messages increases the original mRNA library representation at least 1000 fold with minimal distortion of relative abundance of individual mRNA sequences within the RNA population. The same pre-amplification process may also be known as transcript amplification, linear amplification, or in vitro amplification. Accordingly, it is the 1000 fold linear pre-amplification of the entire mRNA library that is one specific feature of the embodiment of this invention. The single stranded mRNA is annealed in the polyA tail region at the oligo(dT) portion of the T7 promoter containing oligonucleotide. The RNA polymerase creates antisense copies of the entire mRNA library (aRNA). Thus in general, there is at least a 1000 fold increase in the number of copies of mRNA having polyA tails in the entire library, and an associated 1000 fold increase in sensitivity of any particular mRNA sequence type.

For example, the T7 promoter oligonucleotide primer, utilized as the first strand RT primer and a subsequent T7RNAP amplification primer, is composed of 67 bases having a 3′oligo(dT) portion containing a 5′ T7 RNA polymerase promoter sequence having the following base pair order:

(5′-TCTAGTCGACGGCCAGTGAATTGTAATACGACTCACTATAGGGCG
(T)21-3′)

The pre-amplification reaction is completed by a reverse transcription reaction followed by randomly primed DNA polymerase dependant second strand synthesis and finally an overnight incubation with T7RNAP. Subsequently, a portion of this entire reaction mix is used in a PCR reaction analysis, which generates a specific single band amplicon with the appropriately designed gene specific primers (GSP's) of interest or any other appropriate RNA analysis method of choice.

It will be recognized by those skilled in the art that the design and synthesis of gene specific primers will depend upon the particular target sequence to be amplified and can be designed by any means known and accepted in the art. For example, gene specific primers are designed using the NCBI (National Center for Biotechnology Information) BLAST® (Basic Local Alignment Search Tool) software and GenBank human cDNA sequence database. The primers are optimized for annealing temperatures at about 55° C. to 65° C. and shown to produce only DNA-free, RT-PCR dependant single bands from complex mRNA libraries, which are known to be positive for particular mRNA. The complex mRNA libraries are often extracted from normal and cancerous human tissues as well as in vitro cell lines. The designed primers produce desired target sequence specific PCR bands that are all electrophoresed on agarose gels in order to compare design-predicted molecular weights with known standards. Calculations are completed using R_{f} values determined on gel analysis software. The amplicon sequences can be further sequence verified by direct sequencing, blot probing, restriction enzyme mapping, etc.

In order to circumvent the signal-to-noise (S/N) limitation inherent in cDNA array analysis as described above, a novel modification of an RNA polymerase promoter-driven linear amplification strategy was developed. Alternatively, a single tube, multigene RT-PCR analysis system based on universal PCR amplification of multigene-specific reverse transcription of cDNA in a single reaction tube substantially reduces background noise. These two signal-to-noise improvements are specific components embodied in the present invention.

Second strand synthesis of the pre-amplified library is only within selected regions and could include from 1 to 1000 independent regions of interest for a single sample and still maintain the 100% sensitivity from the original library. Second strand synthesis is completed by selective amplification of only those genes of interest. Therefore, gene specific primers (GSP) are designed for second strand synthesis to include only the regions of interest. The regions would include for example, but not limited to, prostate specific antigen (PSA), PSM, CK19, EpCam, AR, HPN, F6, mamoglobin, and/or all the cytokeratins. GSP are designed to incorporate a universal primer on their tail end.

In contrast to prior art where the first strand synthesis is carried out with the set of gene specific primers, part of the novel aspect of this invention is the use of the gene specific primers for only the second strand synthesis without the use of CAPswitch.TM.oligonucleotide, (U.S. Pat. No. 6,352,829). Prior art teaches that the gene specific primers are designed to incorporate an arbitrary anchor sequence at their 5′ ends which includes the CAPswitch oligonucleotide. So surprisingly with the invention herein disclosed, a universal portion of the primers does not include the CAPswitch moiety.

The length of the gene specific primers will typically range from about 15 to 30 nucleotides, while the universal primer portion will typically be about 15 in length.

Reverse transcription of a small portion of the T7 amplified antisense RNA (aRNA) library is performed using cycling conditions known in the art. All RT-PCR results are initially analyzed on 2% agarose gel containing ethidium bromide again according to procedures known in the art.

After amplification of selected portions of the amplified aRNA library, the product is then analyzed in an array format or by any electrophoresis format known in the art.

In addition to the amplification of the second strands after preamplification as described above, a universal PCR multigene amplification can be accomplished in a single tube, incorporating a set of gene specific primers (P1) for simultaneous reverse transcriptase in conjunction with the appropriate set of opposing primers (P2) for simultaneous second strand synthesis. Together, they define both (alpha and beta) termini and form a complete set of gene specific amplicons equaling a GSP multigene panel of interest. The GSP1 and GSP2 priming for both gene specific first and second strand syntheses are conducted with the appropriate enzymes and under conditions of high primer-target annealing specificity, which are know to those skilled in the art. Additional levels and approaches to achieving the appropriate primer specificity can be achieved by using proteins from natural recombination cellular repair mechanisms such as recA. Appropriate application of these repair systems in vitro will enable superior, even absolute, primer template specificity of formation. The template criteria is either mRNA, or mRNA:cDNA heteroduplex, or double stranded duplex cDNA. Furthermore, the innovative idea of utilizing a cell's natural repair mechanisms, as described in the present application, can be applied toward other gene specific primer methods such as the one described below for GSPs-RT subsets for signal to noise shifting enabling cDNA array analysis on rare cell events. Each P1 and P2 primer in any one GSP multigene panel set of PCR primer contains a universal primer sequence at the 5′ terminus which is common to all gene specific P1 's and P2's (or just P1 's and a separate universal sequence which is common to all P2's). In order to control unfavorable side and competitive reactions after second strand synthesis all GSP 1 and 2 are be removed from the desired double stranded cDNA amplicon panel set to eliminate their non-specific impact on down stream processes. Many strategies are possible to those skilled in the art such as molecular size based exclusion offered by Sephadex and Centricon etc, chromatography, solid support selective attachment, single strand specific DNase (Mung Bean, S1, etc.) primer sequence specific strategies such as Uracil-N-Glycosylase (UNG) in combination with DNA oligonucleotide primers that are synthesized with deoxyUridine (U) in place of Thymidine (T). Alternatively, RNA-DNA oligo-primer hybrids could be used in place of DNA-Uracil and similarly be eliminated after first and/or second strand synthesis via DNase-free RNase treatment. The ready availability of Uracil containing DNA-primers combined with the ease of PCR integration of UNG degradation offers an efficient method of eliminating undesirable complex primer interactions. This UNG degradation strategy will produce oligos much smaller than are capable of annealing under chosen PCR annealing temperatures. Following UNG treatment, the cDNA template mixture might also benefit from treatments with DNase-free RNases to eliminate all undesirable side reactions, possibly caused by high complexity RNA. Following UNG treatment (with an optional RNase treatment to eliminate all RNA) the only nucleic acids remaining are hybridized 1^{st} and complimentary 2^{nd} strands forming dsDNA duplexes, which now constitute the sample's available PCR templates. Next, non-UMP containing universal primers (1 or 2 max) are added for the follow-up PCR. The net effect is the capturing of any desired set of mRNA (or DNA minus the RT) sequences with one or 2 PCR compatible high efficiency primers enabling quantitative RT-PCR multigene simultaneous amplification and subsequent analysis in a single tube. Since the primers are universal, they prime each GSP amplicon with the exact same efficiency, eliminating the confounding multiplex GSP primer performance problems. Each GSP defined amplicon with a panel or set of amplicons can have a different predetermined fragment size enabling each GSP sequence to be resolved and identified by its unique Rf value in size-based analysis systems such as vertical and horizontal PAGE and agarose gel electrophoresis, capillary gel electrophoresis, SELDI, MALDI,cDNA arrays, etc. Thus, rapid multigene RNA/DNA panels can be rapidly applied to interrogate large numbers of samples for a diverse set of diagnostic therapeutic and monitoring applications. This method achieves multigene analysis from individual samples of mRNA in a single reaction tube with or without mRNA library preamplification. No preamplification allows only one panel of genes to be analyzed with one assay in one sample. Preamplification adds the advantage of analyzing a single sample in up to 1000 different assays, thus many different panels of genes can be interrogated at different times on one sample. While not limited to any specific method, analysis of the universal PCR panels by cDNA array or capillary gel electrophoresis (CGE) is a preferred methodology.

Thus a critical feature differentiating the present invention from conventional technologies of the prior art is the improvement in signal to noise by selective amplification of rare target mRNA species, making this method a novel development over existing multivariate mRNA analysis. Known multivariate analysis systems, for example multiplex RT-PCR, can substantially change signal to noise, however the challenges of designing and optimizing meaningful multiplex systems has rendered them generally impractical especially for more than two target subsets in a reaction vessel.

This invention also utilizes the high signal to noise improvement to select representative transcripts, and amplifies in one reaction vial the entire set of target sequence(s) to be detected.

Thus while the invention is not limited to the following specific use, a set(s) of representative gene specific primers can be used to generate target gene subset(s) found in known disease states. The representative set is will include at least two different target genes that are indicative of the disease state of interest. For each reaction vial, the number of sets of gene specific primers will be determined by the disease state and the known characteristics that would define the disease state.

The following examples are provided to exemplify the practicality of the disclosed invention and to demonstrate the impact of the invention on diagnostic technology. These examples are not intended to limit the scope of the invention. In addition, the disclosures of each patent, patent application, and publication cited or described in this document are incorporated herein by reference in the entirety. Throughout these examples, molecular cloning reactions, and other standard recombinant molecular biology techniques, were carried out according to methods described in Maniatis et al., Molecular Cloning-A Laboratory Manual, 2^{nd} ed., Cold Spring Harbor Press (1989) (hereinafter, “Maniatis et al.”), and Current Protocols in Molecular Biology, Wiley, 2002, using commercially available reagents, except where otherwise noted.

### EXAMPLE 1

Isolation of Cytoplasmic RNA

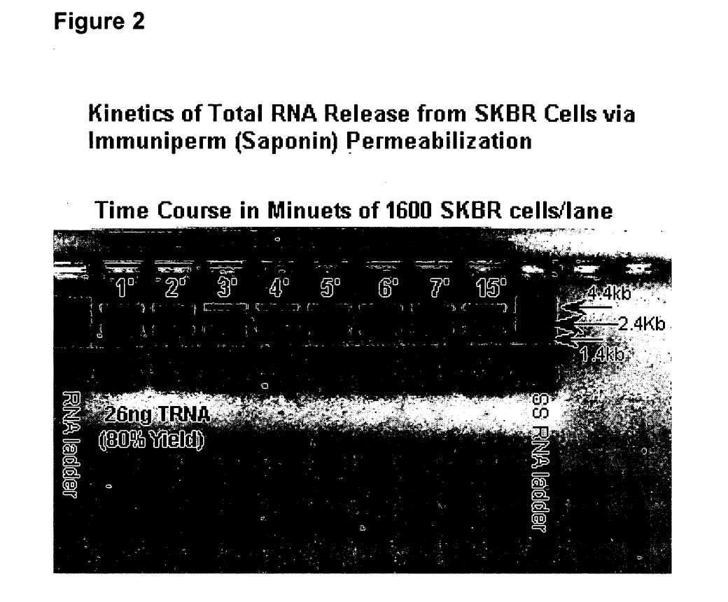

The supernatant obtained from ferrofluid selected unfixed cells that are permeabilized with Immuniperm, a phosphate buffered solution containing 0.05% saponin and 0.1% sodium azide was found to contain greater than 80% of the cellular total RNA residing in the cytoplasm of the cells. The RNA isolated from this supernatant showed no evidence of degradation as judged by native and denaturing agarose gel electrophoresis and ethidium bromide staining. This supernatant solution, which is normally discarded after intracellular staining of the ferrofluid selected cells, was unexpectedly found to contain the RNA in an intact or undegraded full-length form thus providing an mRNA profile of the same cells that were also used for morphologic analysis. FIG. 2 illustrates these findings showing that total RNA release occurs in less than one minute and that about 95% of the cytoplasmic total RNA can be readily and reproducibly isolated.

In dramatic contrast, the RNA isolated from cells of the breast cancer cell line SKBR3 using the conventional process, i.e. isolation by a commercially available phenol based RNA lysis buffer Trizol® Reagent (Gibco BRL, Gaithersburg, Md., Cat # 10296), completely lyses and homogenizes the entire cellular structures, thereby also resulting in the liberation of the genomic and mitochondrial DNA, and the cytoplasmic, mitochondrial and nuclear RNA. Examination of the cell pellet from Immuniperm (saponin) treated SKBR3 cells selected with an EpCAM ferrofluid (see Example 2 for a detailed procedure) showed the presence of nearly 100% of the genomic and mitochondrial DNAs, and the nuclear and mitochondrial RNA which amounts to approximately 15-20% of all the cellular total RNA expected from the same number of non-permeabilized whole cells. About 95% of expected cytoplasmic RNA was recovered intact from the Immuniperm supernatant layer.

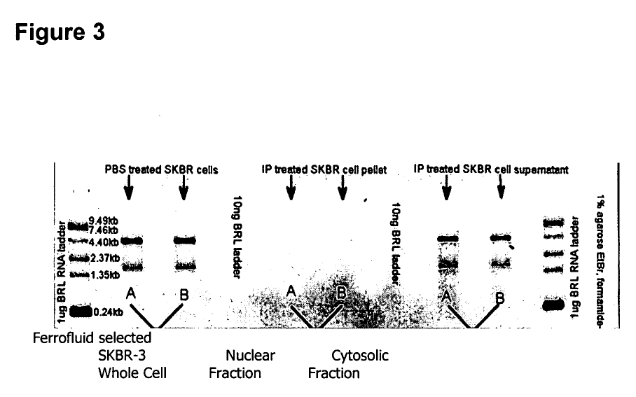

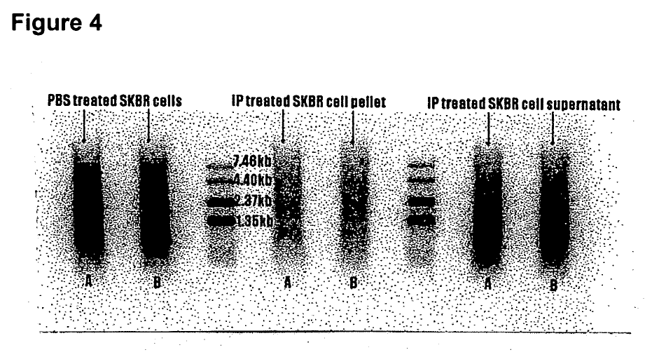

As shown in FIG. 3, duplicate tubes containing about 250,000 cells of the breast cancer cell line SKBR3 were first immunomagnetically enriched and then incubated in the absence (PBS only) and presence of Immuniperm (+IP) for 15 minutes at room temperature. The Immuniperm treated permeabilized cells were then separated by centrifugation for 5 minutes at 800×g RCF as a visible permeabilized cell pellet. The Immuniperm supernatant fraction containing all the cytoplasmic soluble components was transferred to a second tube. Total RNA isolated from whole untreated cells, the Immuniperm treated permeabilized cell pellets, and the Immuniperm treated cell supernatant fractions were isolated using the RNeasy® (Qiagen Inc., Valencia, Calif.) silica binding method. These total RNA fractions were then DNase treated to remove trace amounts of DNA. Spectrophotometric quantitation of the DNased RNA fractions yielded an average of 20 picograms of total RNA/cell for whole cells (=100%), 4 picograms of total RNA/permeabilized cell from the pellet fraction (20%), and 16 picograms of total RNA/cell from the supernatant fraction (80%). From these three, DNased total RNA fractions, 2.5×10⁴ cell equivalents of mass were then formamide-denatured and electrophoresed through a 1% agarosse gel followed by staining with ethidium bromide, as shown in FIG. 3. Quantitation by agarose gel densitometry agreed with the spectrophotometric recovery values. The gel images in FIG. 3 also show that the rRNA are full-length and have high integrity as evidenced by the relative ratios of the 4.4 kb ssRNA co-migrating 28S rRNA bands compared to the 2 kb ssRNA marker co-migrating with the 18S rRNA bands. In general, the observed relative ratios of 28S rRNA to 18S rRNA of approximately 2 are an excellent indication of mRNA integrity as was further demonstrated by Northern blotting this gel and probing it with oligo(dT) as shown in FIG. 4. Literature values for the percent of total RNA contributed from the nucleus range from 15 to 25%. The Immuniperm® treated cell fractions containing 20% RNA are thus consistent with the published nuclear contribution.

In conclusion, Immuniperm-based permeabilization was unexpectedly shown to provide complete separation of nuclear and cytoplasmic total RNA with nearly 100% of the cytosolic total RNA readily recoverable in the supernatants of the Immuniperm treated unfixed cells. Furthermore, the nuclear fraction of total RNA surprisingly was found to remain intact in the resultant permeabilized cell structure following Immuniperm treatment.

Using poly-A tail hybridization, mRNA portions of the RNA derived from the two Immuniperm® cell fractions were evaluated against whole cells by Northern blot transfer of the denatured RNA from the gel shown in FIG. 3 to a positively charged nylon filter. Oligo(dT) 25-mer probe was labeled with ³²P and poly nucleotide kinase then hybridized to the Northern blot. Single-stranded RNA size ladders containing poly-A tails were used as markers enabling the formation of a molecular weight banding ladder for relative qualitative sizing of mRNA populations. The oligo(dT) hybridization results in FIG. 4 shows that no significant differences in size ranges were observed for the mRNA libraries between the three samples.

Comparative quantitative phosphor image analyses of the entire mRNA-blotted regions (i.e. the entire dT hybrid signals) from whole cell mRNA libraries were nearly identical to the sum totals from the Immuniperm treated permeabilized cell pellets (nuclear fraction) plus the Immuniperm-derived cytosolic fractions. Furthermore, the cell fraction percentages of mRNA from the dT-probe signals, determined by phosphorimaging, are identical to the 28S/18S rRNA percentages determined from the agarose gel image densitometry analyses. These data demonstrate that both the Immuniperm-derived cytosolic total RNA and its mRNA component are quantitatively isolated, exhibit high integrity, and are full-length. The release of Immuniperm-derived mRNA is not limited by transcript size since nearly 100% of the cytosolic mRNA is retrievable from the Immuniperm supernatant, and the integrity of rRNA 28S/18S is indicative of full retention of mRNA integrity.

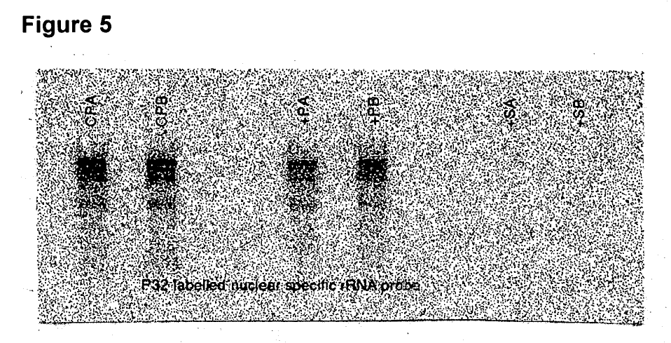

The Northern blot shown in FIG. 4 was stripped and re-probed with nuclear specific precursor rRNA probe. FIG. 5 shows that Immuniperm treatment of cells achieves a complete separation of nuclear and cytosolic total RNA populations. Thus, the nuclear membrane structure remains intact during the Immuniperm treatment and the nucleus retains all its soluble components.

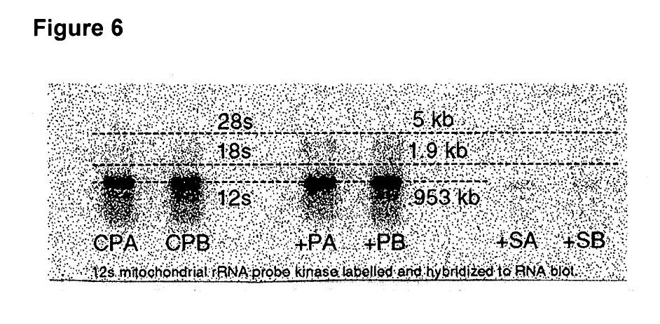

The Northern blot in FIG. 5 was stripped and re-probed with a mitochondrion-specific 12s rRNA probe. The results, shown in FIG. 6, demonstrate that Immuniperm treatment of the cells achieves complete separation of mitochondrial and cytosolic RNA populations. Thus, the mitochondrial membrane structures remain intact during the Immuniperm treatment.

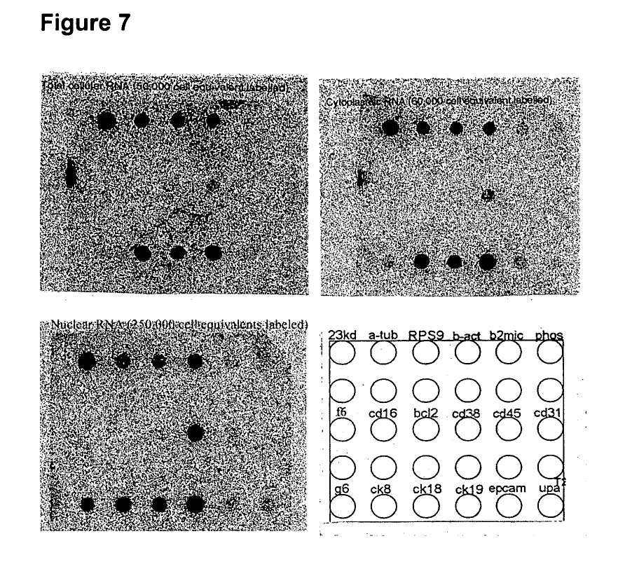

The same total RNA stocks solutions, which were used to generate the images in FIGS. 3-6, were further used to generate ³²P-labeled first strand cDNA libraries of equal masses and specific activities. These three labeled first strand library probes were hybridized to obtain separate but identically prepared cDNA array dot blots as shown in FIG. 7. Here the objective was to evaluate the mRNA relative abundances represented in each Immuniperm derived cell fraction by comparing the relative proportions of each cDNA hybrid signal pattern for each imaging filter. The randomly selected cDNA gene array identities on the template are shown in FIG. 7. The uppermost row of dots are a set of housekeeping genes designated by the abbreviations 23 kd=23 kilodalton protein, a-tub=alpha tubulin, b-act=beta actin, b2mic=beta-2-microglobulin, phos=phospholipase A2. The second row is designated as f6=alpha-1-globin, CD16=cluster determinant 16, CD12, CD38, CD45, and CD31. The bottom row consists of general epithelial specific genes designated by g6=macrophage inhibitory cytokine 1, CK8=cytokeratin 8, CK 18=cytokeratin 18, CK 19=cytokeratin 19, EpCam=epithelial cell adhesion molecule, uPA=urokinase plasminogen activator. Qualitative and quantitative phosphorimaging comparisons of each fraction showed no significant differences in the relative abundances of the genes on the array. Interestingly, these data shown that the relative mRNA abundance from whole cells is roughly equal to its cytosolic fraction, which in turn equals its nuclear fraction, where total RNA mass can vary from about 20% in the nucleus to about 80% in the cytosol. The respective lengths of the transcripts in this cDNA array varies from 1 to 5 kb, again reinforcing the Northern blot finding that no size bias is seen in the release mRNA from Immuniperm treated cells. The identical relative representation patterns in FIG. 7 also unexpectedly demonstrate that the Immuniperm®-derived mRNA is as effective a reverse transcriptase template for first strand synthesis as the whole cell mRNA derived by traditional methods.

Overall these data show the unexpected findings that Immuniperm-derived cytosolic RNA yields approximately 80% of the mass of all the cellular total RNA in the entire homogenized cell which is essentially >95% of all cytosolic total RNA, that it is full length, and that it has the same efficiency of reverse transcription as total RNA isolated by traditional phenol and silica extraction methods. Thus, Immuniperm-derived cytosolic total RNA and its accompanying heteronuclear RNA components are equally effective templates in all conventional downstream RNA analysis methods when compared to the traditional whole cell high-quality RNA isolation methods.

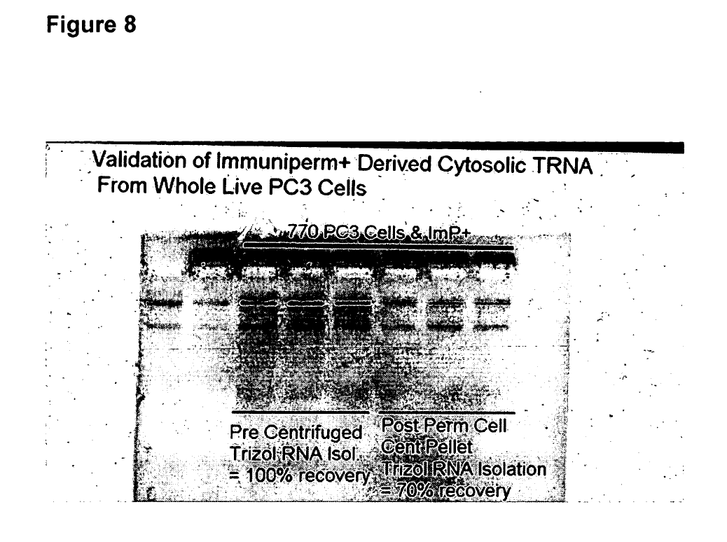

FIG. 8 shows the gel image of the cytosolic RNA obtained by treating about 770 PC-3 cells with Immuniperm. The data show that Immuniperm-derived cytosolic mRNA from lower cell numbers give the same proportions of RNA as were demonstrated in FIGS. 3, 4, 5, 6, and 7. Therefore, Immuniperm-mediated cell release of cytosolic RNA is not cell number dependent.

### EXAMPLE 2

Isolation of Circulating Tumor Cells from Peripheral Blood

Isolation of circulating tumor cells from peripheral blood followed by cell analysis by flowcytometry and gene expression analysis by RT-PCR can be performed as follows: EDTA-anticoagulated blood (7.5 ml) is transferred into a 15 ml conical tube and 6.5 ml of System Buffer (PBS also containing 0.05% sodium azide, Cat #7001, Immunicon Corp., Huntingdon Valley, Pa.) is added. The tube is securely capped and mixed by inverting several times. The blood-buffer mixture is centrifuged at 800×g for 10 minutes at room temperature. The supernatant is carefully removed by aspiration taking care not to disturb the buffy coat layer. Some supernatant can be left in the tube. The aspirated supernatant can be discarded. AB Buffer (System Buffer containing streptavidin as a reversible aggregation reagent, Immunicon Corp., Huntingdon Valley, Pa.) is added to the tube to a final volume of 10 ml. The tube is capped and mixed by inverting several times. VU/desthiobiotin EpCAM ferrofluid particles (Immunomagnetic nanoparticles coupled to anti-EpCAM monoclonal antibody also conjugated to desthiobiotin for biotin-reversible aggregation with streptavidin, Immunicon Corp., Huntingdon Valley, Pa.) are resuspended by gently inverting the vial several times. To the sample in the AB buffer is added 100 ul of VU/desthiobiotin EpCAM ferrofluid and the tube mixed by inverting several times. Shaking should be avoided to avoid foaming. The tube is immediately inserted into the QMS17 (Cat. # AS017, Immunicon Corp., Huntingdon Valley, Pa.) magnetic separator and let stand for 10 minutes. The tube is removed from the separator and its contents resuspended by inverting the tube several times. The tube is inserted into the QMS17 magnetic separator again and let stand for 10 minutes. The tube is removed from the separator and its contents resuspended by inverting the tube several times. The cap is removed and the tube is placed in the QMS17 separator for an additional 20 minutes. With the tube inside the QMS17, the cell-buffer mixture is carefully aspirated using a Pasteur pipette and the aspirated supernatant is discarded. Immediately thereafter, the tube is removed from the separator and 3 ml of System Buffer is added. The magnetically collected cells are resuspended by brief vortexing. The liquid should rise up the tube during vortexing so that cells near the top are washed down. The uncapped tube is again placed in the QMS17 separator for 10 minutes and the supernatant is aspirated with a Pasteur pipette. The aspirated supernatant is discarded. The magnetically collected cells are resuspended by vortexing in 200 ul of Immuniperm/RNase inhibitor (Permeabilization reagent, Immunicon Corp., Huntingdon Valley, Pa.) also containing RNase inhibitor, RNase OUT, Cat. # 10777019, Invitrogen, Rockville, Md.). The liquid should rise up the tube during vortexing so that all cells are washed down. Antibodies such as, for example, monoclonal anti-cytokeratin antibody (C11-PE, 0.25 ug) (cocktail of antibodies recognizing cytokeratins 4, 5, 6, 8, 10, 13, 18 conjugated to R-Phycoerythrin; Immunicon Corp., Huntingdon Valley, Pa.) in a 25 ul volume and 10 ul of CD45 PerCP (Pan anti-leukocyte marker, Cat. # 347464, Becton Dickinson, San Jose, Calif.) or any other suitable antibodies can be added and mixed by vortexing. After 15 minutes of incubation, the sample is gently agitated by lightly tapping the bottom of the tube. The tube is returned to the QMS17 for 5 minutes. The supernatant is gently aspirated and the Immuniperm-RNA fraction transferred to an appropriately labeled tube.

### EXAMPLE 3

Cell Analysis of Circulating Tumor Cells from Peripheral Blood

The cells from Example 2 are resuspended in 200 ul of CellFix (PBS based buffer containing biotin as a de-aggregation reagent and cell preservative components, Immunicon Corp., Huntingdon Valley, Pa.) and incubated for 5 minutes. The sample is transferred to a 12×75 mm flow tube and 300 ul of PBS are combined, followed by the addition of the nucleic acid dye thioflavin T (Sigma # T3516, 10 ul) and about 10 ul of fluorescent beads (10,000 beads; Flow-Set Fluorospheres, Cat. # 6607007 Coulter, Miami, Fla.). The sample is mixed by vortexing. Preferably, the fluorescent beads tube is mixed by vortexing before pipetting the beads. The sample is then analyzed on a flowcytometer.

### EXAMPLE 4

Gene Expression Analysis of Circulating Tumor Cells From Peripheral Blood

The poly(A)+ mRNA is isolated using magnetic oligo(dT) labeled beads (Dynabeads® mRNA Direct® (Micro Kit, Dynal, Prod. # 610.21, New Hyde Park, N.Y.). Alternatively, total RNA can be isolated by using any other appropriate means to those skilled in the art such as silica binding, polymer binding, and more traditional phenol extractions like Trizol® Reagent (GibcoBRL, Cat # 10296). Genomic DNA is eliminated by treatment with DNase enzyme such as DNase I (GibcoBRL). An enzyme mix composed of 2:1 of 10× DNase I (1 U/:1), 1:1 of RNase inhibitor (cloned), 5:1 of dH₂O, and 10:1 of RNA or control (250 ng Genomic DNA) is prepared. The enzyme mix is incubated at 37° C. for 20 minutes. The DNased RNA is re-purified by magnetic oligo(dT) labeled beads or Trizol® isolation and resuspended in 10:1 RNase-free water. The activity of DNase enzyme is confirmed by running the control genomic DNA (+/−DNase treatment) on a 2% agarose gel with ethidium bromide staining.

Specific mRNA sequences can be amplified using rTth (Thermos thermophilis) RT-PCR. A master mix composed of 10:1 of 5× EZ Buffer, 1.5:1 of dATP, 1.5:1 of dCTP, 1.5:1 of dGTP, 1.25:1 of dUTP, 5:1 of Mn++ (25 mM), 2:1 of rTth (2.5 U/:1), 0.5:1 of UNG (1U/:1), 12.25:1 of dH₂O, 2.25:1 of sense primer, and 2.25:1 of anti-sense primer can be prepared for reverse transcription of specific mRNA species. A 40:1 volume of Master Mix is added to the sample tube containing 10:1 of DNased RNA and corresponding negative control tubes containing 10:1 of H₂O. PCR thermocycling is carried out for 40 cycles as follows: 50° C. for 2 minutes (pre-PCR), 62°/65° C. for 30 minutes (pre-PCR), 95° C. for 1 minute (pre-PCR), 94° C. for 15 seconds (PCR), 62°/65° C. for 30 seconds (PCR), and 62°/65° C. for 7 minutes (post-PCR). After the thermocycling is completed, the sample tube is immediately placed in a −20° C. block for 2 minutes. After completion, the sample tube is placed in a 4° C. block until gel analysis will be performed. A volume of 20:1 is run on a 2% agarose gel with ethidium bromide staining. Qualitative and quantitative gene expression measurements of specific mRNA transcripts are made by examination of the gel image using a UV transilluminator and an alpha imager for the presence of the amplicon at the expected molecular weight.

Various modifications of the invention, in addition to those described herein, will be apparent to those skilled in the art from the foregoing descriptions. Such modifications are also intended to fall within the scope of the appended claims.

### EXAMPLE 5

Isolation and Analysis of Proteins from Circulating Tumor Cells In Peripheral Blood

The supernatant obtained from about one million ferrofluid-selected SKBR3 cells that have been permeabilized with Immuniperm, a phosphate buffered solution containing 0.05% saponin and 0.1% sodium azide, also contains, in addition to the nucleic acid components analyzed in Examples 1 to 4, released soluble cytosolic proteins residing in the cytoplasm of the cells. The soluble proteins in this supernatant solution and the insoluble proteins remaining in or on the surface of the cells thus provide a means for determining the total protein expression profile or proteomics profile of the cells as well as the cellular morphology.

Firstly, the fraction of total cytosolic soluble protein liberated from the cytoplasm due to Immuniperm treatment is determined relative to the total amount of protein liberated from a duplicate cell preparations treated with NP-40, a surfactant that is the preferred reagent for total cytosol protein release from cells. Both treated cell preparations are freed from membrane debris via centrifugation or magnetic separation prior to determination of total soluble proteins by conventional methods, such as the spectrophotometric Lowry and Bradford methods.

Secondly, aliquots of the two sample preparations are electrophoresed in a 4 to 20% gradient SDS polyacrylamide gel to (a) determine the molecular weight cut-off for Immuniperm-derived cytosolic proteins and (b) compare the protein banding patterns and relative quantities of protein per band in the two preparations.

Thirdly, aliquots are further analyzed by 2D electrophoresis and conventionally stained or detectably labeled to provide “fingerprint” information on sizes and isoelectric points of the proteins in the two fractions based on the qualitative and quantitative spot patterns of identifiable and unidentified components. The derived information generates proteomic expression profiles of the relative and absolute protein expression patterns in the cytosolic and total protein compartments of normal and transformed cell populations.

### EXAMPLE 6

Gene Specifically Primed (GSP)RNA Polymerase Based Amplification of mRNA Library Subsets Enabling Diagnostic Formats With Inherent Signal-to-Noise (S/N) Limitations Such as cDNA Array Analysis of Rare Cell Events and Rare Transcripts

After RNA isolation, several RNA analysis methods can be applied. Traditional RT-PCR or the more desirable quantitative versions can be applied however they are generally considered a poor use of individual samples as these samples yield very small amounts of starting material.

As a consequence, clinical sensitivity is compromised for multigene analysis. Thus, unamplified mRNA/cDNA libraries can only be analyzed one time for only one gene without compromising clinical (and maximum technical) sensitivity. With individual samples being scarce, several higher throughput methods were developed.

Here, we show that it is highly desirable to be able to measure the expression level of multiple genes (from 2 up to 1000s) simultaneously via high throughput formats such as in a micorarray format all from only one reaction tube. This is accomplished without reducing the workload significantly and loss of sensitivity. A significant obstacle to single tube cDNA micorarray analysis for rare cell event and their rare mRNA samples is their inherent unfavorable S/N ratio in the starting mRNA sample.

For radiolabeled cDNA arrays, these limits originate from (a) the lower limit of the target copy number detectable in the solution phase (approximately 5×10⁵) when one specific known target is spiked into the (b) maximum amount of labeled non-specific (background noise) targets (20 ng=2×10¹¹ library of randomly labeled target molecules) that can be hybridized to a nylon filter array system at one time without increasing the background filter (solid support) noise component of the S/N ratio.

For Immunomagnetically enriched samples, significant background noise mRNA is due to the presence of WBC's which are unavoidably carried over during the enrichment process. One solution is to shift the S/N ratio up to 1000 fold in favor of the desired rare mRNA from the rare cells by performing a second round of RNA polymerase amplification (RNAPA) selecting only a subset of predetermined genes.

This GSP subset RNAPA selection process is reduced to practice in this example using a model system that reflects typical WBC mRNA copy number ratios found in clinical samples (10 ng total RNA in approximately 5000 WBC)/CTC (0.5 ng total RNA in approximately 50 CTC). In an equivalent aliquot of this starting sample stock composed of 50 CTC, the number by real-time quantitative RT-PCR was determined for all detectable mRNA in prostate specific antigen (PSA=2650), prostate specific membrane antigen (PSM=1750), androgen receptor (AR=100) and epithelial cell adhesion molecule (EpCAM=1163) as show on Table 1. The starting WBC mRNA total copy number proportional to the non-specific background noise was approximately 10⁸ to 10⁹. For this particular example, the starting total RNA/mRNA was subjected first to one round of amplification which increased proportionately all the mRNA species approximately equal as determined by real-time quantitative RT-PCR (Table 1). Subsequently, a 25 ng aliquot of the first round amplified aRNA was subjected to a second round of GSP subset RNAPA, shifting the signal-to-noise of the 4 GSP targets as described below (Table 1).

In the second round GSP RNAPA, a key selection step occurs during the single RT reaction forming simultaneous first strands only for a predetermined mRNA library subset of which the gene specific RT primers are included. In this example, the subset of GSP RT primers were for the above 4 mRNA (PSA, PSM, AR, EpCAM). GSP-RT selective first strand synthesis is followed by synthesis of the complimentary second strand using the appropriate DNA polymerase and oligo(dT) primer bearing a T7RNAP promoter, thus creating a selective set of double stranded DNA templates T7RNAP ready.

Thus, the desired subsets of RNAPA enabled templates have been selected via GSP first and second strand synthesis. At this point, all remaining RNA is degraded by exposing the second strand reaction mix with a cocktail of DNase-Free RNases. Alternatively, any remaining single stranded RNA and any extraneous (non-poly U/poly A dependent) single stranded cDNA which was formed during dT dependent second strand synthesis can be eliminated by single-strand-specific nucleases such as Mung Bean Nuclease. Then, double-stranded cDNA template subsets are purified by phenol extraction and/or silica binding. The selected set of RNAPA ready templates are RNAP amplified overnight to yield an approximately 1000 fold increase of only 4 genes of interest in S/N shifting over the other possible templates such as the F6 (alpha 1 globin sequence) which represents WBC mRNA derived noise. Table 1 shows the results of real-time quantitative RT-PCR for these 4 genes of interest throughout the process including subsequent GSP-second round S/N shifting where F6 is defined as alpha 1 globin sequence found in this system to be highly abundant in WBC and not detectable in epithelial cells. These results clearly show that four of the GSP targets selected increased an average of 844-fold while the non-targeted F6 WBC noise only increased 5.9 fold. Thus when dividing the increase GSP target signal by the F6 WBC noise, the final signal to noise improvement for each GSP target was derived. It is important to note that further improvements would be expected by employing modifications such as Mung Bean Nuclease and GSP-RT primer sequence selection/optimization as mentioned above.

| | tRNA starting | Total copy no. after | Total copy no. after | | GSP S/N |
|---|---|---|---|---|---|
| Gene | copy no. | 1^{st} round aRNA | GSP 2^{nd} round | 1^{st}:2^{nd} | (F6) |
| PSA | 2650 | 2.10 × 10⁷ | 3.00 × 10¹⁰ | 1238 | 210 |
| PSM | 1750 | 7.80 × 10⁶ | 4.03 × 10⁹ | 517 | 87 |
| AR | 100 | 64200 | 2.90 × 10⁷ | 458 | 78 |
| EpCAM | 375 | 5.20 × 10⁶ | 6.05 × 10⁹ | 1163 | 197 |
| F6 | NA | 1.40 × 10¹⁰ | 8.30 × 10¹⁰ | 5.9 | 1 |

### EXAMPLE 7

Proteinase and Nucleophile Based Recovery of Cellular RNA From Fixed Samples Yields High Quality RNA Template For Down Stream RT Dependent Analysis

Surprisingly for samples exposed to aldehyde and urea based stabilizers or fixatives, Cyto-Chex™ and other formaldehyde and formaldehyde-urea derivative based fixatives stabilize approximately 100% of full-length total RNA, mRNA and other nucleic acids in all cells found in whole blood when compared to matched non-fixed controls. Intact RNA, stabilized as macromolecular complex, changes its RNA chemical characteristics and is unaffected by current traditional cell lysis and chaotropic salt based RNA isolation methods such as phenol extraction, silica binding and oligo(dT) hybridization.

These macromolecular complexes (both covalent and noncovalent) are dissociated and reversed through combinations of enzymatic digestion and/or chemical nucleophile agent incubations. Consequently, nucleic acids are liberated, enabling isolation of nearly 100% of original DNA and RNA libraries fully intact. These fixative-derived RNA libraries provide high quality templates for reverse transcriptase (RT) dependant formation of first strand cDNA.

These fixative recovered RNA are combined with an aRNA preamplification or universal PCR methods described in the present application for comprehensive analysis down stream or for general functional enablement of total and mRNA library.

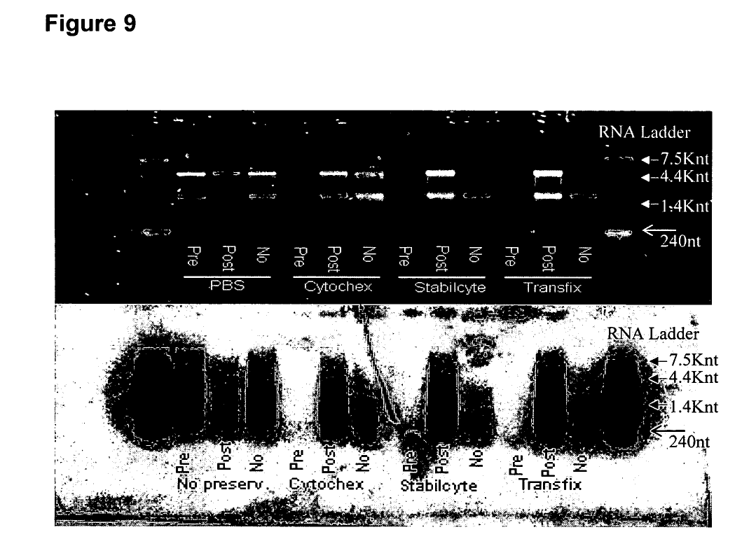

FIG. 9 demonstrates that Cyto-Chex™ performs like other aldehyde based fixatives. Upon fixative exposure for 24 hours, less than 1% of the mRNA and a disproportionate amount of 18S-rRNA are recoverable (approximately 10%). Even when extreme chaotropic salt denaturation chemistries are applied (i.e. GITC and Phenol, silica or (dT) hybridization, BRL's Trizol Reagent, Qiagen's RNA mini silica binding and Dynal's Dynabeads mRNA Direct oligo (dT) poly (A)⁺ kits) recovery is extremely low.

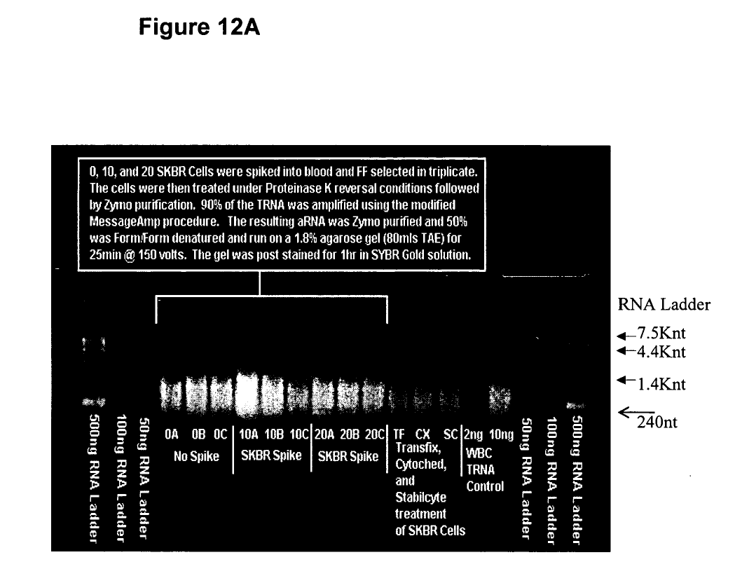

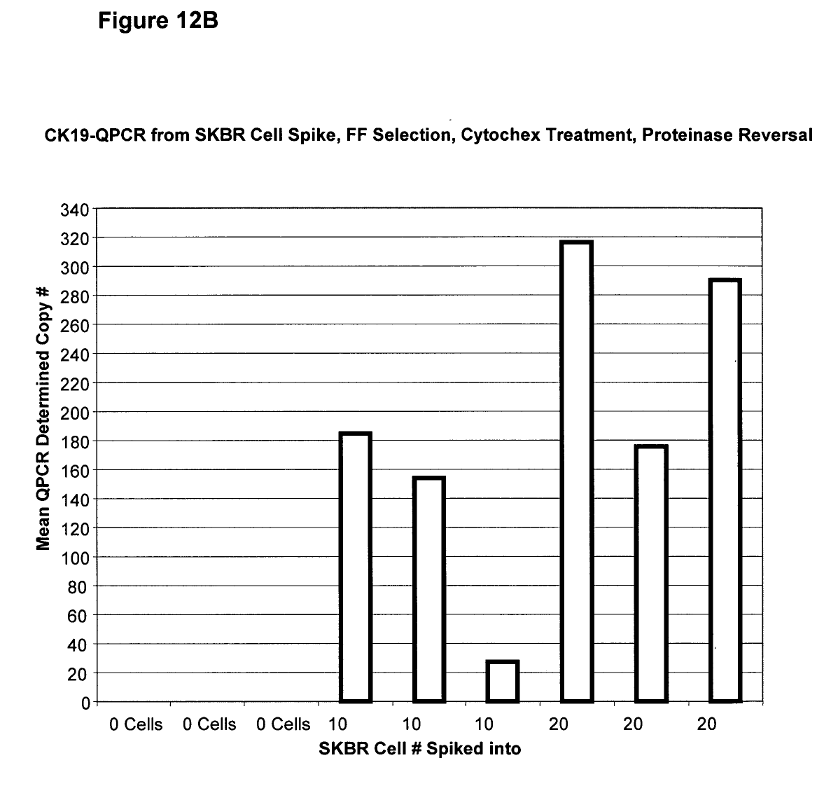

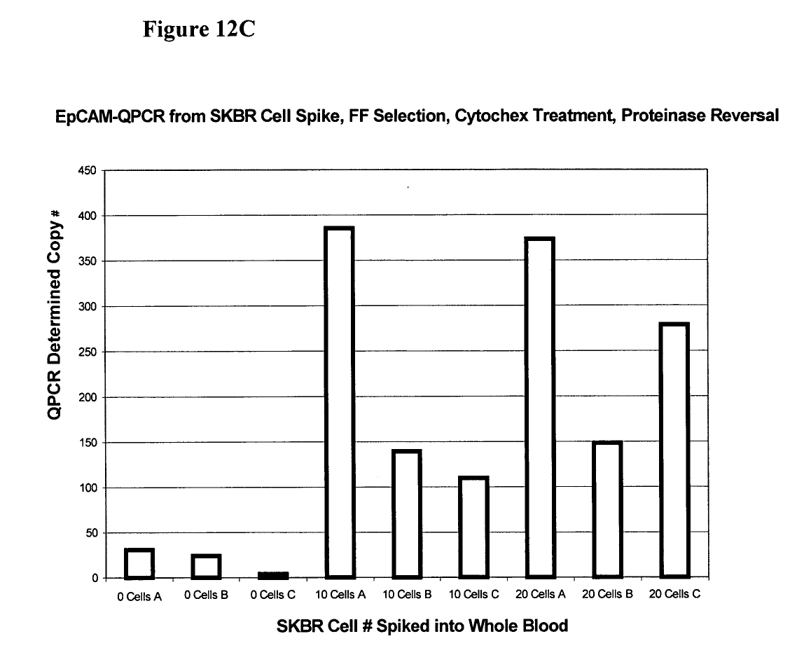

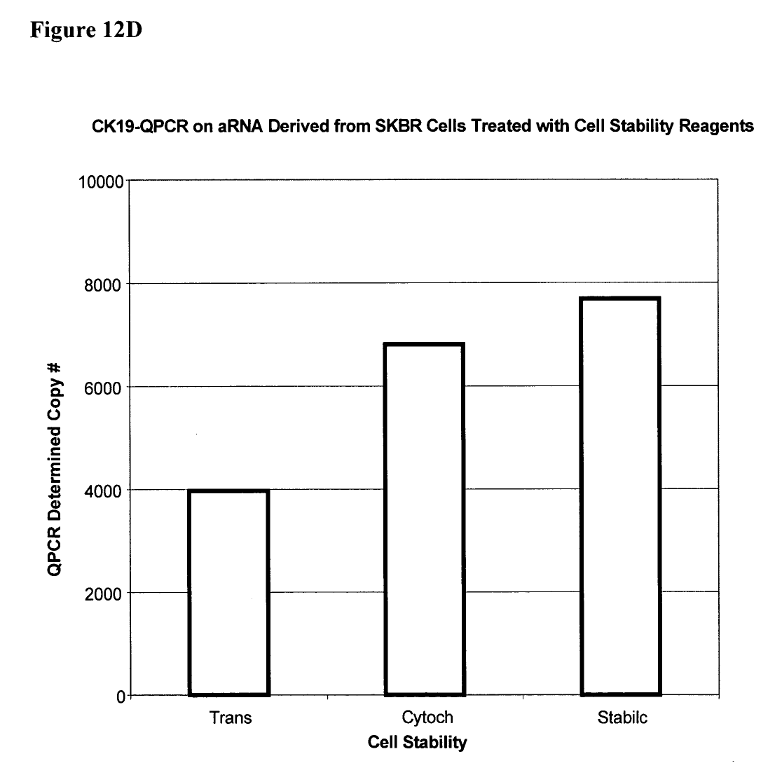

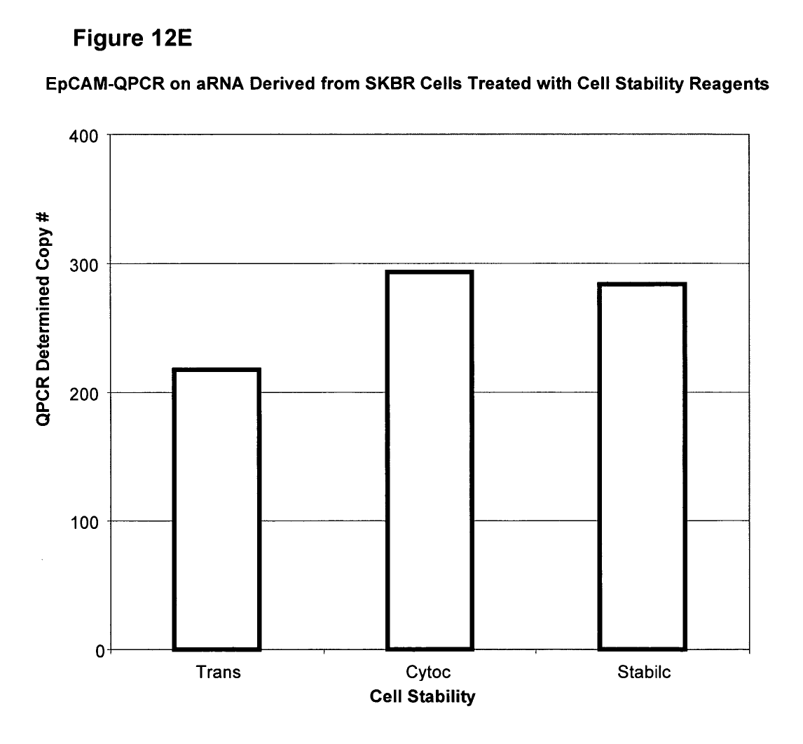

Surprisingly, treatments with proteinases, such as proteinase K, and nucleophiles like Tris base, which removes the majority of proteins and polypeptides covalently linked to other macromolecules including nucleic acids, restored sufficient nucleic acid chemistry properties to enable recovery of greater than 90% of the original total RNA and mRNA in a fully intact state. A 25 ng aliquot of aRNA (FIG. 12A), derived from a mass normalized aliquot of the total RNA found in FIG. 9, was subjected to quantitative RT-PCR comparative analysis for both CK 19 and EpCAM. This comparison showed a 3.8 and 3.9 fold lower copy number from fixative derived RNA relative to non-fixed RNA. This is understood as the current proteinase K treatment for restoring at least 25% of the maximal RT-template activity for RT-PCR analysis.

The restoration of 25% of the maximal RT-template activity via this fixation-recovery system is reproducible when different operators conduct the same procedure and analyze Percoll derived white blood cells for specific mRNA (alpha globin) via quantitative RT-PCR relative to non-fixed matched samples.

Furthermore, it is known that the Transfix™ formulation used here achieves a 0.1% final concentration of paraformaldehyde fixative per unit volume blood.

Since the loss and recovery behavior of Cyto-Chex™ exposed RNA is identical to Transfix™ and other aldehydes shown below it is highly likely that the formaldehyde donor component of the formaldehyde urea derivative components found in the formula of Cyto-Chex™ and Stabilcyte™ are responsible for the covalent linkages of nucleic acids to protein.

Formaldehyde-urea derivatives in the presence of numerous macromolecular nucleophiles found in biological systems (i.e. proteins and nucleic acids) leads to an increase in the rate of dissociation of these derivatives.

Dissociation occurs in close proximity to biological nucleophile complexes, possibly regulatory proteins specifically associated with RNA that leads to covalent linkages. These linkages and associations are then removed and reversed by subsequent proteinase and stronger nucleophile treatment. The fact that a known cross-linking agent, Transfix™ yields full-length high integrity mRNA libraries from 24 hr stabilized whole blood cells demonstrates that all aldehyde based stabilizers will yield nucleic acids of similar high quality. Thus resulting in a reproducible yield of nucleic acids after preservation and recovery.

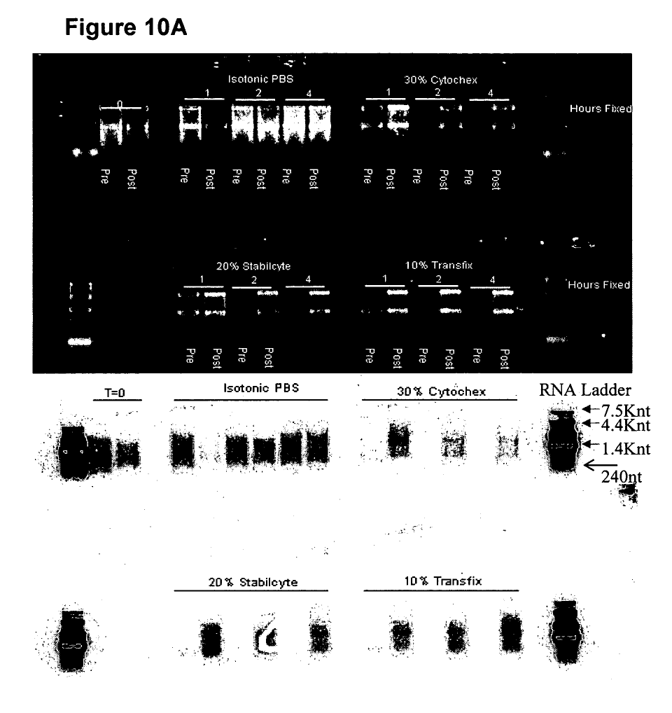

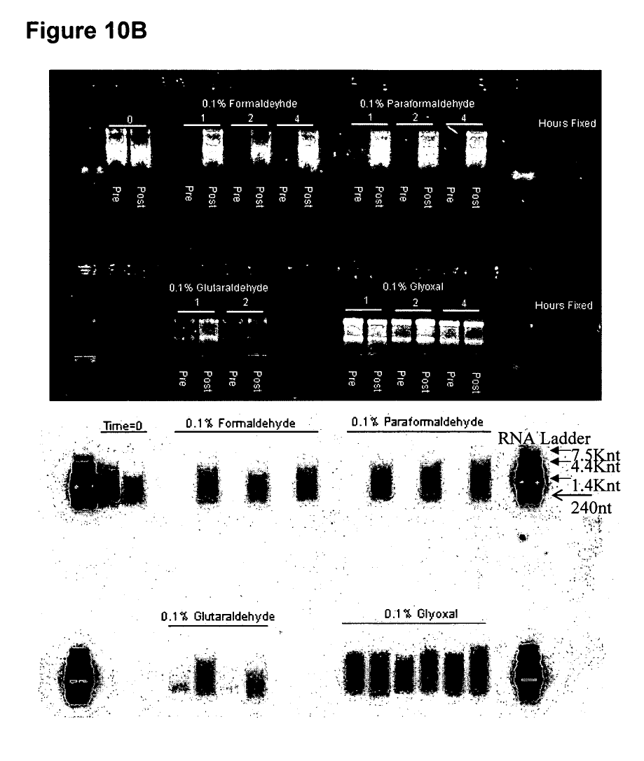

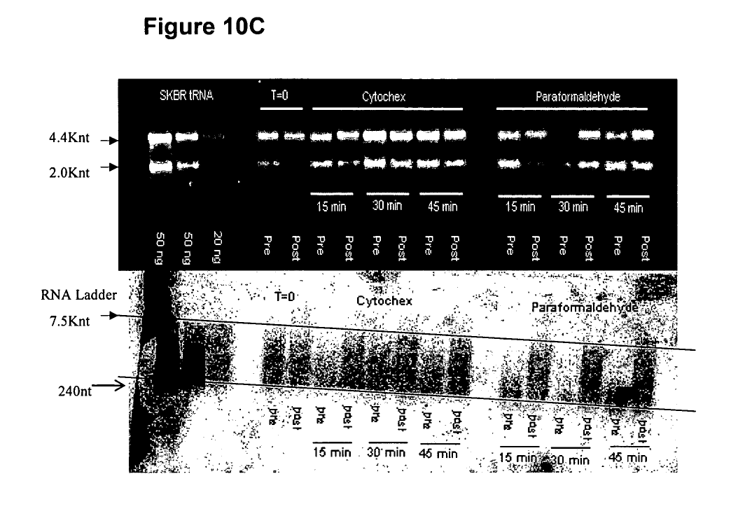

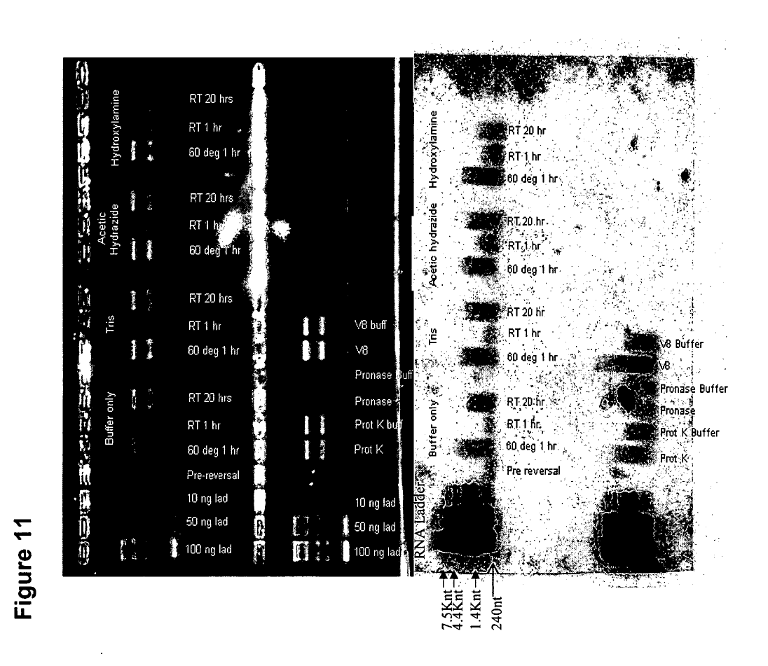

Analysis of 90-100% of the total RNA and their corresponding mRNA libraries are possible with these and most other aldehyde and/or urea derivative fixatives as is further shown in FIGS. 10A, 10B and 10C below. In addition, most of these types of fixed nucleic acids can be recovered via combinations of proteinase and nucleophile cocktails heated at high temperatures such as 60° C. as shown in FIG. 11.

These results show that heating the fixed RNA in buffers alone at high temperatures for one hour yields a portion of the mRNA library. This high temperature recovery effect has been previously shown for formalin fixed, paraffin embedded tissue RNA retrieval, however nowhere has this result been reported in whole blood. Furthermore, the quality and quantity of the mRNA library recovered in the present application has not be obtained even those reports using formalin fixed, paraffin embedded tissue RNA retrieval.

Comparing this mRNA library to those recovered using the other nucleophiles (Tris, acetic hydrazide and hydroxylamine), mRNA transcript size distribution proportions for each nucleophile are different even though none of the samples shows RNA degradation. This suggests that different types of mRNA sequences are retrievable (i.e. different types of formaldehyde modifications are reversed) by specific nucleophiles and incubation conditions. The various enzymes used also show different proportions recovered (FIG. 11, bottom of gel).

The fact that proteinase K digestion alone restores 25% of maximum RT-template activity combined with the observation that different fixative reversal agents yield different proportions of mRNA libraries strongly supports the notion that significantly improved recoveries by employing combinations of nucleophiles and enzymes are tangible.

To demonstrate the feasibility of mRNA diagnostic applications for cancer in particular using relatively non-invasive peripheral blood model, FIG. 12A shows the resultant relative quality and quantity of aRNA from a single T7RNAP preamplification of the total RNA/mRNA isolated in triplicate of 10 and 20 SKBR3 cell spikes into 7.5 ml peripheral blood after stabilization with Cyto-Chex™ for 24 hours at room temperature. Each of these replicates was then immunomagnetically enriched from which the cell lysate was treated with proteinase reversal conditions and followed by silica binding total RNA isolation. Normalized equivalents of aRNA were used in quantitative RT-PCR reactions for two specific genes, CK19 and EpCAM. The results of which are shown in FIGS. 12B and 12C. As can be seen from FIGS. 12B and 12C, all spikes, as measured by CK19, were strongly positive relative to the donor-matched triplicate (no-spike controls). Similarly, EpCAM has the same 100% positive score for all spikes in spite of the ectopic (endogenous) and extremely low level of expression seen in this donor's triplicate no-spike controls. In fact, this low level of EpCAM mRNA detection by RT-PCR is commonly seen for this sequence and is not due to false positive PCR contamination.

FIGS. 12D and 12E shows the varying mRNA RT-template quality that is derived from the three different aldehyde based fixatives, Cyto-Chex™ Stabilcyte™ and Transfix™. As shown, Transfix™ yields an mRNA template, which might be of a slightly lower RT quality than either Cyto-Chex™ or Stabilcyte™. Furthermore when these data are normalized to the number of spiked cells and compared to unmodified cells at time=0 in the exact same flask of SKBR3 cells, the difference between fixed verses non-fixed CK19 and EpCAM mRNA is only 4 fold (FIG. 9 derived RNA data not shown). This is interpreted to mean that the 24-hour stabilization process here combined with proteinase K recovery alone and aRNA amplification yielded 25% of the possible template quality for RT. Further, the reason for lower than 100% RT quality is likely due to aldehyde modification, which proteinase K alone cannot remove.

Thus, combinations of proteinase and nucleophile cocktails will significantly improve the RT-quality of these templates beyond 25% as demonstrated in these experiments. For comparison with the relevant literature under similar conditions, studies evaluating the same parameters of RT RNA template quality derived from formalin fixed paraformaldehyde embedded tissues show a 13 to 60-fold reduction in RT-template quality relative to non-fixed tissues (Godfrey, et al., Quantitative mRNA Expression Analysis from Formalin-Fixed, Paraffin-Embedded Tissues Using 5′ Nuclease Quantitative Reverse Transcription-Polymerase Chain Reaction, J. of Molecular Diagnostics, 2:84-91 (2000)). Consequently, the 4-fold reduction provides a significant improvement over prior art.

FIGS. 12A, 12B, 12C, 12D, and 12E confirm a reproducible, product viable, procedure for blood RNA sample preservation and comprehensive analysis of circulating epithelial cells, which are most likely cancer cells. The high level of mRNA preservation is amenable to qualitative analysis that can detect the presence of single cell spiked into 7.5 ml of blood for any mRNA that exists in that cell within at least 50 mRNA molecules/cell (i.e. only 50 copies/sample).

In summary the rates and types of covalent fixations in whole blood vary according to the type of fixative. Likewise, the rates and types of covalent fixative reversal or recovery will vary according to the type or combination of proteinases and nucleophiles used. The rate of fixation will be a critical issue for applications where the half-lives of mRNAs of interest are faster then the rate of fixation. Both the forward fixation and the reversal recovery reactions (processes) can be optimized further yielding yet higher quality and quantity of RNA. However, the current quality and quantity of the RNA stabilized and recovered is demonstrated here in blood to be far superior to anything previously shown.

### EXAMPLE 8

Enrichment and Analysis of mRNA from CTC in Fresh Non-Fixed Blood

Human blood was isolated from 9 patients with advanced hormone refractory prostate cancer (HRPC) and 13 healthy volunteers and assessed for gene expression mRNA specific to circulating epithelial cells.

Patients

Blood was drawn into 10 ml EDTA Vacutainer™ tubes (Becton-Dickinson, N.J.) of 9 patients with advanced hormone refractory prostate cancer (HRPC) and 13 healthy volunteers, 7 male (age ranging from 24 to 73, mean 45), and 6 female (age ranging from 27 to 61, mean 39). Of the 9 HRPC patients, 2 patients had 5 longitudinal blood samples, 1 had 4 samples, 3 had 2 samples, and 3 were 1 sample time points. Patients' age range was 60-81 years (mean 74), and their initial diagnosis was 2-10 years prior to the study. Serial samples from three patients who were undergoing treatment with taxol/estramustine and/or Lupron were prepared and analyzed as a longitudinal series. Patients and healthy volunteers signed an informed consent under an approved research study.

Target Cell Isolation

Blood samples were kept at room temperature and processed within 2-3 hours after collection unless otherwise indicated. 15 ml of blood were divided into 7.5 ml aliquots and transferred to disposable tubes with an internal diameter of 17 mm (Fisher Scientific) and centrifuged at 800 g for 10 min with the brake off. Phosphate buffered saline (PBS) with bovine serum albumin (BSA) was added to bring the volume up to 10 ml and the sample was mixed by inversion. The Mab VU-1D9 recognizing the epithelial cell adhesion molecule (EpCAM) is broadly reactive with tissue of epithelial cell origin and coupled to magnetic nanoparticles (ferrofluids, Immunicon, Huntingdon Valley, Pa.).

To increase the magnetic loading of the EpCAM-positive cells and decrease the variability in capture efficiency due to differences in the EpCAM density on the cell surface, desthiobiotin are coupled to EpCAM-labeled magnetic nanoparticles to form CA-EpCAM, as described in Applications No. 09/351,515 and No. 09/702,188, both of which are incorporated by reference herein. CA-EpCAM ferrofluid and a buffer containing streptavidin are then added to the sample to achieve this increase in the magnetic labeling of the cells. Desthiobiotin on the CA-EpCAM ferrofluid is subsequently displaced by biotin, which is contained in the permeabilization buffer described below. Thereby reversing the cross linking between the CA-EpCAM ferrofluid particles. The sample was immediately placed in a quadrupole magnetic separator for 10 min (QMS17, Immunicon). After 10 min, the tube was removed from the separator, inverted 5 times, and returned to the magnetic separator for an additional 10 min. This step was repeated once more and the tubes were returned to the separator for 20 min. After separation, the supernatant was aspirated and discarded. The tube was removed from the magnetic separator, resuspended with 3 ml of phosphate buffered saline (PBS) containing bovine serum albumin (BSA), and the fraction collected from the walls of the vessel.

Two 7.5 ml aliquots from each sample were processed separately. One aliquot was prepared and analyzed by flowcytometry (EXAMPLE 12), and the RNA from the other aliquot was analyzed as described.

Nucleotide Purification and Amplification

One manner to utilize the invention in the preferred embodiment is to first purify the nucleotide sample. Here, total RNA or mRNA is isolated from the enriched cell population.

Isolation can be accomplished by any means known in the art that is able to keep the mRNA intact and prevent degradation. For example, the enriched circulating tumor cells from duplicate blood samples were lysed in 100 ul of Trizol reagent (BRL) or 100 ul of RNA Extraction Buffer (ZYMO Research) and the vortex-homogenized sample was stored at −80° C. until RNA was used.

Homogenates were used to isolate total RNA according to manufacturers' instructions. Briefly, total RNA was treated with DNase I. DNase activity was verified to produce no ethidium bromide gel detectable genomic DNA after DNase treatment. DNased RNA was cleaned with repeated Trizol isolation procedure. One tenth of the resultant total RNA was electrophoresed on a 1% agarose gel along total RNA mass and size standards, and then Northern blotted, hybridized with an equimolar mixture of ribosomal 18S and 28S oligos. The resultant hybrid blot was labeled with ³²P, phosphor imaged (Packard Cyclone) and analyzed to determine RNA integrity and mass. The remaining total RNA mass values (90%) from each sample were then designated as that sample's 7.5 ml blood donor equivalent of total RNA, 1.5% of which was calculated to be mRNA.

### EXAMPLE 9

Flowcytometric Analysis in Patients After Immunomagnetic Selection

Flowcytometric analysis of leukocytes taken from human blood was assessed for gene expression in circulating epithelial cells. Isolated cells were prepared as described then resuspended in 200 ul permeabilization buffer containing biotin (Immunicon Corporation) to which monoclonal antibody (Mab)-fluorochrome conjugates were added at saturating conditions. The monoclonal antibodies consisted of a Phycoerythrin (PE) conjugated anti-cytokeratin monoclonal antibody (Mab C11) recognizing cytokeratins 4,6,8,10,13, and 18 (Immunicon) and peridinin chlorophyll protein (PerCP)-labeled anti-CD45 (Hle-1, BDIS, San Jose, Calif.). After incubating the cells with the Mab for 15 min, 2 ml of cell buffer (PBS, 1% BSA, 50 mM EDTA was added and the cell suspension was magnetically separated for 10 min. After discarding the non-separated suspension, the collected cells were resuspendened in 0.5 ml of PBS to which the nucleic acid dye used in Procount System™ was added (Procount, BDIS). In addition, 10,000 fluorescent counting beads were added to the suspension to verify the analyzed sample volume (Flow-Set Fluorospheres, Coulter, Miami, Fla.)

Samples were analyzed on a FACSCalibur flowcytometer equipped with a 488 nm Argon ion laser (BDIS). Data acquisition was performed with CellQuest™ (BDIS) using a threshold on the fluorescence of the nucleic acid dye. The acquisition was halted after 8000 beads or 80% of the sample was analyzed. Multiparameter data analysis was performed on the listmode data (Paint-A-Gate™, BDIS). Analysis criteria included size defined by forward light scatter, granularity defined by orthogonal light scatter. Positive staining with a nucleic acid stain and the PE-labeled Pan anticytokeratin Mab C11 (CK4, 5, 6, 8, 10, 13, and 18) combined with staining with the PerCP-labeled anti-CD45 Mab was used for differential CTC/WBC fluorescent staining and analysis. CTC's were identified by the presence of nucleic acid dye and cytokeratin antigens, coupled with the absence of CD45 staining. For each sample, the number of events present in the region, typical for epithelial cells, was multiplied by 1.25 to account for the sample volume not analyzed by flowcytometry.

In healthy, non-cancer donor samples, the leukocytes carried over from the immunomagnetic selection ranged from 655 to 5,560 (median 4,350; mean 1,759). In HRPC patient samples, the leukocytes carried-over ranged from 813 to 92,000 (median 4,350; mean 12,300). Blood samples from healthy, non-cancer control group, 7 male and 6 female, showed no CTC whereas in the blood samples from HRPC showed a CTC range of 4-283 in 7.5 ml of blood.

### EXAMPLE 10

Quantitation of mRNA Transcript from the Amplified Library

Normalization of mRNA/aRNA mass was determined by first quantitating the total RNA mass isolated from each immunomagnetically enriched 7.5 ml blood sample volume. This was accomplished by Northern blotting 10% of each sample's total RNA, followed by 28S plus 18S radiolabeled oligo probe hybridization, and in parallel with known total RNA mass cell line standards. This was followed by phosphoimage quantitation (Cyclone, Packard Instruments). Resultant total RNA masses were defined as 1 Donor Sample Equivalent of mRNA=1 Donor Sample Equivalent of aRNA:
[(total RNA mass)×(1.5% mRNA)]/3*=1 Donor Sample Equivalent aRNA
*(Average molecular weight of aRNA libraries was found to be 3-fold lower than the unamplified mRNA molecular weight)

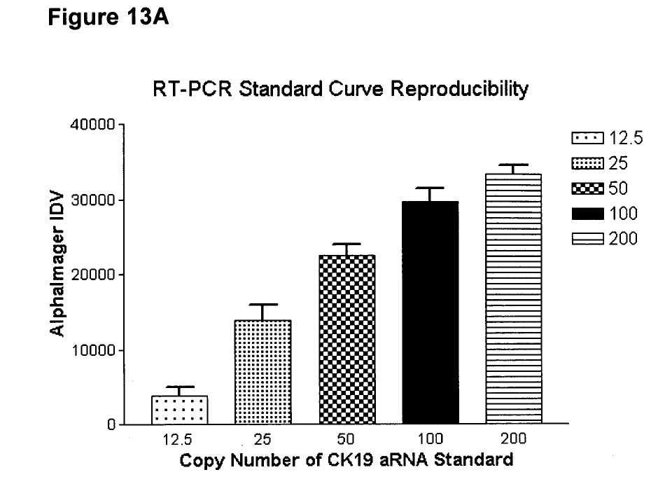

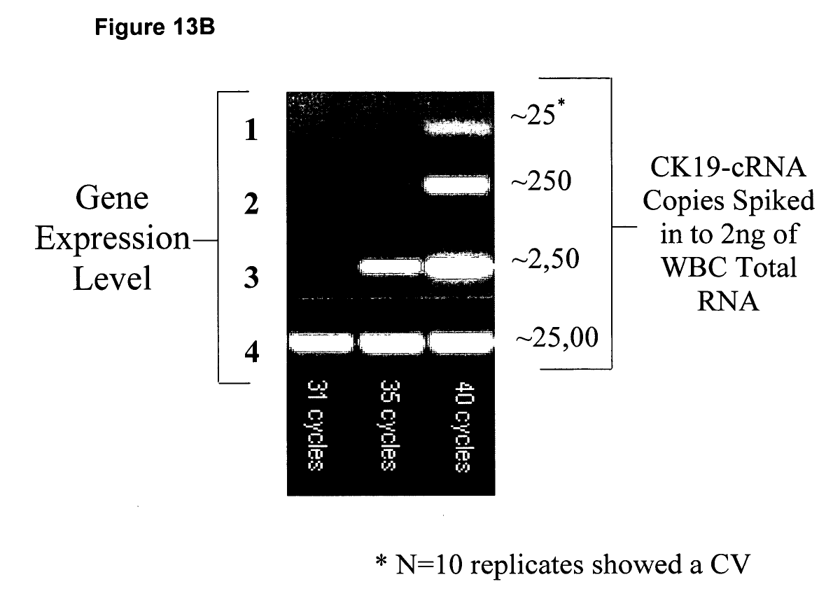

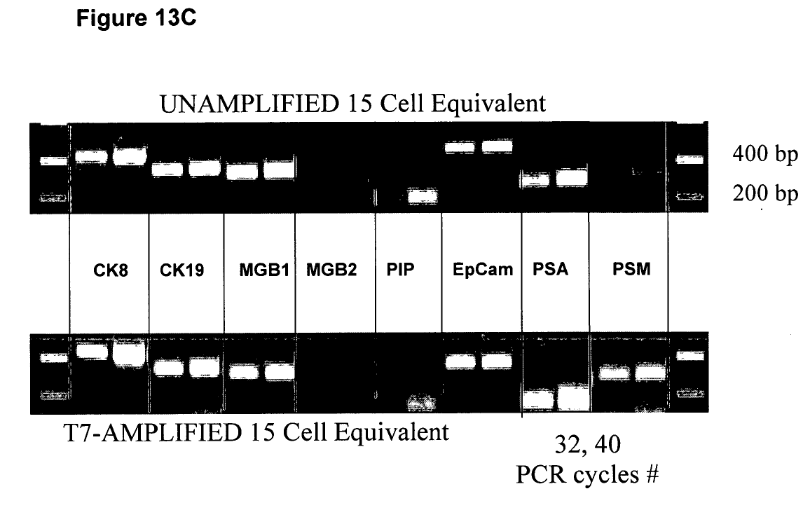

Relative gene expression levels of 0, 1, 2, 3, and 4 were assigned to unknowns based the amplified product's agarose gel kinetics curve band intensity containing the CK19 in vitro transcribed RNA construct (CK19-cRNA) standard of known copy numbers. This CK19-cRNA standard contained the 3′-most 800 bases of CK19 wild type mRNA sequence. Standard CK19-cRNA curves covered a 1000 fold dynamic range were run in triplicates at 20,000; 2,000; 200; 100; 50; 25; and 12.5 copies each spiked into 2 ng total RNA isolated from Percoll-derived WBC. Standard kinetics curves run for 40 cycles showed linear signal response plotting band intensity against RNA copy number between 13-200 copies CK19-cRNA transcript [see FIGS. 13A and 13B]. The external standard curve had a maximum CV of 27% for any standard analyzed in triplicate. For multivariate gene analysis, comparisons were made to CK19 external standard curves and relative gene expression levels 0-4 were assigned: non-detectable
1—approximately 25-50 copies CK19,
2—approximately 250-500 copies CK19,
3—approximately 2,500-5,000 copies CK19, and
4—greater than 25,000 copies CK19.

FIG. 13B shows typical banding intensities that correspond to the approximate copy number of CK19 cRNA on the standard curve, and were used to assign relative gene expression levels 1-4.

The CTC enumeration and gene transcript expression profiles were determined using 23 different PCR amplification products from Ep-CAM immunomagnetically enriched blood samples of 13 healthy donors and 9 HRPC patients. Microarrays were not effective for analyzing these types of samples due to the signal to noise incompatibility derived from the WBC which are nonspecifically carried over during the immunomagnetic enrichment process. The ratio of CTC specific signal to WBC carry-over noise in these samples ranges from 1 to 1000 CTC per 10³ to 10⁴ WBC. These microarray limitations were overcome by incorporating a 10,000 fold preamplification step using 90% of the entire mRNA library from each immunomagnetically enriched blood sample, followed by multigene RT-PCR analysis in place of the arrays. This innovation provides enough starting material for several hundred individual PCR reactions to be performed with each 7.5 ml blood sample. Thus, one is enabled to perform individual patient CTC multivariate RT-PCR profile analysis without compromising assay sensitivity or clinical sensitivity for each mRNA member of each CTC mRNA library.

After the volume normalization procedure described above, the remaining 90% total RNA from each sample was reverse transcribed (RT) using a SMART PCR cDNA synthesis kit, but using the 67 base oligo(dT) primer described above. The reaction was incubated at 42° C. for 90 min. The entire 10 ul RT was transferred into a 50 ul PCR reaction using the Advantage cDNA PCR kit (Clontech) and subject to PCR with the P1-SMART primer and P2-T7 18 base primer: (5′-TCTAGTCGACGGCCAGTGAATT-3′) using a PE-9600 and thermal cycling program; 95° C. for 1 min., 10 cycles of 95° C. for 15 sec., 65° C. for 1 sec., 68° C. for 6 min.; followed by 20 min at 72° C. The entire PCR reaction volume was loaded on a Sephadex G-50 Quick Spin (TE) column (Roche Diagnostics) and the eluate was generated according to the manufacturer's instructions.

The eluate was vacuum concentrated to 3 ul on a Vacufuge (Eppendorf) at 60° C. for approximately 30 min. T7 RNA polymerase transcript amplification reactions that produced representative libraries of aRNA were assembled using AmpliScribe kit (Epicenter Technologies) according to manufacturer's instructions in a 20 ul volume and incubated at 37° C. for 6-12 hours. Repeating the Trizol isolation procedure further cleaned up the RNA transcription reaction.

RNA size standards, RNA mass standards, and one tenth of the transcription reaction products from each sample were formamide denatured at 65° C. for 15 min., loaded on 2% agarose gel, run for 15 min at 5 volts/cm, and post-stained with SYBR Gold™ (Molecular Probes) for one hour prior to gel image densitometry using Alphalmager™ (Alpha Innotech Corp.). The mass of each transcript library was determined.

Gene specific primers were designed as describe. All primer sets were designed to amplify specific gene target cDNA within the 3′-most 500 bases (averaging 344 bp and ranging 226-513 bp long) of each specific gene target. To avoid amplification of genomic DNA, all RNA samples were treated as described wit Dnase. Table 1 shows the primer pairs for each amplicon analyzed by relative RT-PCR. Forward primer P1 is shown as the upper sequence in the respective primer pair. Reverse primer is the lower sequence. All sequences are written 5′→3′.

TABLE I
Primer Pairs

| | | P1 distance | | Length of |
|---|---|---|---|---|
| Gene | accession no. | from 3′ end | Sequence of selected primer pair | Amplicon |
| Alpha 1-globin | V00491 | 580 | AAGACCTACTTCCCGCACTT | 451 |
| | | | TATTTGGAGGTCAGCACGGT | |
| AR | NM_000044 | 513 | ATCTCTGTGCAAGTGCCCAAGAT | 207 |
| | | | CAGGAACATGTTCATGACAGACTGT | |
| BCL2 | XM_008738 | 440 | GCAAGAGTGACAGTGGATTGCAT | 330 |
| | | | CTAATGGTGGCCAACTGGAGACT | |
| CK05 | NM_000424 | 353 | AGTCACTGCCTTCCAAGTGCAGCAA | 212 |
| | | | GGAAACCTGAAGGCTGATTTGAAGCAG | |
| CK08 | M34225 | 429 | GTGGTTTGAGCTCGGCCTATGG | 275 |
| | | | CCAGTGCTACCCTGCATAGCG | |
| CK10 | NM_000421 | 305 | GACGGTAGAGTTCTTTCATCTACGGTTG | 196 |
| | | | GGAAACCACAAAACACCTTGTAGACACC | |
| CK18 | NM_000224 | 331 | ACCTTGAGTCAGAGCTGGCACAGA | 275 |
| | | | GCTTCTGCTGGCTTAATGCCTCAG | |
| CK19 | NM_002276 | 320 | CATATCCAGGCGCTGATCAGCG | 228 |
| | | | CAAAGGACAGCAGAAGCCCCAG | |
| DD3 | AF103908 | 2160 | GTAAGCCTGGGATGTGAAGCAAAGG | 260 |
| | | | GAACCCTAAAGTGGCTCACAAGAGTG | |
| EGFR | NM_005228 | 265 | CCTGTAACCTGACTGGTTAACAGCAG | 200 |
| | | | GGCTCTGACTGATCTGGGAGTCA | |
| EpCam | M33011 | 432 | GAGATGCATAGGGAACTCAATGC | 275 |
| | | | ACGATGGAGTCCAAGTTCTGGAT | |
| ER-a | NM_000125 | 382 | AGCAGGTGCCTGAGACACAGA | 328 |
| | | | TCGAGCATCCCGCTGGATTCTT | |
| ER-b | NM_001437 | 323 | GGAAGCTGGCTCACTTGCTGAA | 232 |
| | | | GAAGCACGTGGGCATTCAGCAT | |
| Her2Neu | M11730 | 426 | TCGTTGGAAGAGGAACAGCACTG | 266 |
| | | | AGCCTGGATACTGACACCATTGC | |
| HK2 | XM_008996 | 334 | CACACCATGCAGGATGACAT | 190 |
| | | | GCATTCCACAAGGTTCTCAG | |
| MDR1 | AF016535 | 263 | AGTGTCCAGGCTGGAACAAAG | 215 |
| | | | CTCCACTTGATGATGTCTCTCAC | |
| MGB1 | XM_006409 | 408 | CTCCCAGCACTGCTACGCAGGC | 340 |
| | | | GACATAAGAAAGAGAAGGTGTGGT | |
| MGB2 | NM_002407 | 420 | CTCCTCCTGCACTGCTATGCAGAT | 264 |
| | | | ACACCAAATGCTGTCGTACACTGTATGCA | |
| Mic1 | AF019770 | 352 | CTACAATCCCATGGTGCTCA | 289 |
| | | | ACACAGTTCCATCAGACCAG | |
| MMP2 | NM_004530 | 332 | ACTGCTGGCTGCCTTAGAACCTT | 216 |
| | | | GAGAAGAGACTCGGTAGGGACAT | |
| MMP9 | NM_004994 | 327 | CGTCTTCCAGTACCGAGAGAAAG | 215 |
| | | | TGTATCCGGCAAACTGGCTCCTT | |
| MRP | L05628 | 328 | GGTGATCGTCTTGGACAAAGGAG | 300 |
| | | | TCTTCACAGCCAGTTCCAGGCAG | |
| MUC1 | J05582 | 361 | AACGGTGGCAGCAGCCTCTCTTA | 238 |
| | | | GCTTCCACACACTGAGAAGTGTCCG | |
| NKX3A | NM_006167 | 284 | GGAAGTTCAGCCATCAGAAGTAC | 246 |
| | | | GGTATGGGTAGTAAGGATAG | |
| p53 | AF307851 | 334 | CTGCCTCAGCCTCCGGAGTAGCT | 243 |
| | | | GTGGGGTGAAAATGCAGATGTGC | |
| PIP | J03460 | 226 | CAGAACTGTGCAAATTGCAGCCGTC | 201 |
| | | | AGACCACAGCAGAAATTCCAGCCAAG | |
| PSA | M26663 | 236 | TGAAGCACTGAGCAGAAGCTGGA | 236 |
| | | | GAGGGTTGTCTGGAGGACTTCAA | |
| PSGR | AF311306 | 425 | ACACCGCTTTGGAAACAGCCTTCATC | 281 |
| | | | GTACTGATGTGCTTATGGGCAACTGG | |
| PSM | M99487 | 459 | AGTTCAGTGAGAGACTCCAGGAC | 286 |
| | | | CTGCACTGTGAAGGCTGCAACAT | |
| TERT | NM_003219 | 347 | AGCACACCTGCCGTCTTCACTT | 234 |
| | | | GGCACACCTTTGGTCACTCCAA | |
| Timp1 | NM_003254 | 302 | CCAAGACCTACACTGTTGGCTGT | 240 |
| | | | ACTGTGCAGGCTTCAGTTCCACT | |
| Timp2 | NM_003255 | 325 | TGGGCTGCGAGTGCAAGATCAC | 219 |
| | | | CTGCTTATGGGTCCTCGATGTC | |
| Topo2a | NM_001067 | 356 | GCCATCCACTTCTGATGATTCTG | 244 |
| | | | ACCAGTCTTGGGCTTGGTAAGA | |
| Topo2b | NM_001068 | 366 | AAGCCCAAGAGAGCCCCAAAAC | 279 |
| | | | TGGCAGAGAAGGTGGCTCAGTA | |
| TROP2 | X77753 | 334 | TGTTGCTACTCTGGTGTGTCCCAAG | 245 |
| | | | CTGGGATTCAAAGGAGGTACAGCTC | |
| uPA | NM_002658 | 387 | TGGGCTGTGAGTGTAAGTGTGAG | 285 |
| | | | CACCCAGTGAGGATTGGATGAAC | |

Reverse transcription (RT) was performed on 25 nanograms of the T7 preamplified aRNA library using random 9 mer 50 ng, 1 ul Superscript™ (BRL) according to manufacturer's instructions. The RT was incubated at 25° C. for 10 min, 37° C. for 10 min, 42° C. for 20 min, 50° C. for 60 min. Ten donor equivalents per sample of aRNA (ranging 50-1300 pg) were used in each subsequent 50 ul PCR reaction containing 1 unit of platinum taq (BRL). Individual PCR curves were generated from each single PCR reaction tube by aliquoting 15 ul at 31, 35, and 40 cycles during the thermal cycling program: 95° C. for 1 min, and 31,35, and 40 cycles of 95° C. for 1 sec, 65° C. for 1 sec, 72° C. for 1 min; followed by 20 min at 72° C. using PE-9600 thermalcycler (or thermocycler??). Each amplicon within each PCR batch included a cell line cDNA amplification positive control, and a master mixed PCR reagent amplification negative control that contained all components except for the cDNA sample. All RT-PCR results were analyzed on a 2% agarose gel containing ethidium bromide in parallel with BRL low mass molecular weight and spot densitometry analysis with AlphaEase™ software version 5.04.

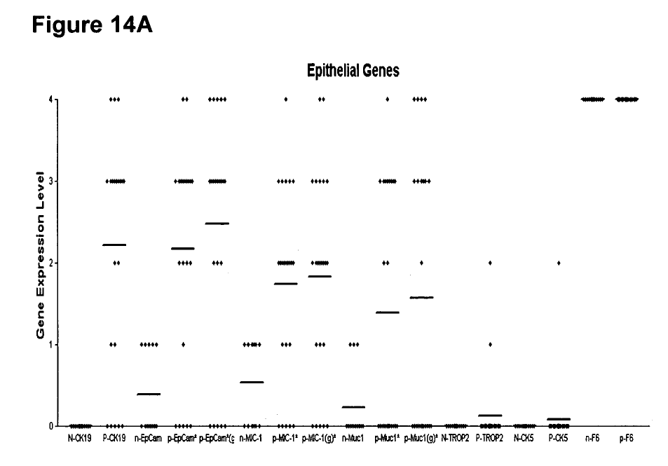

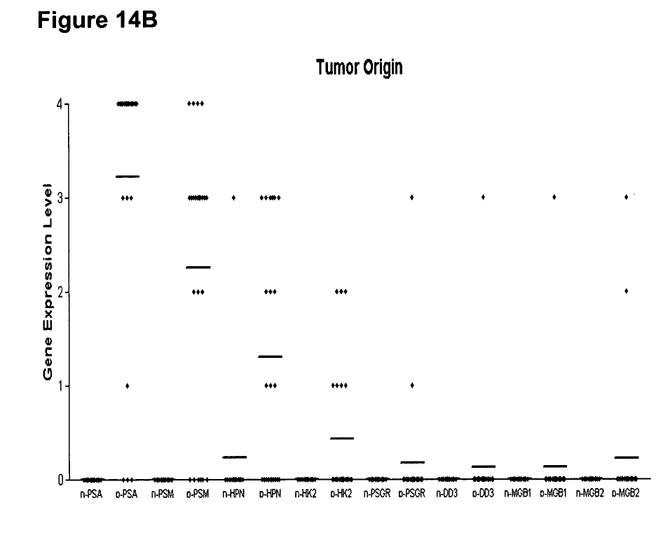

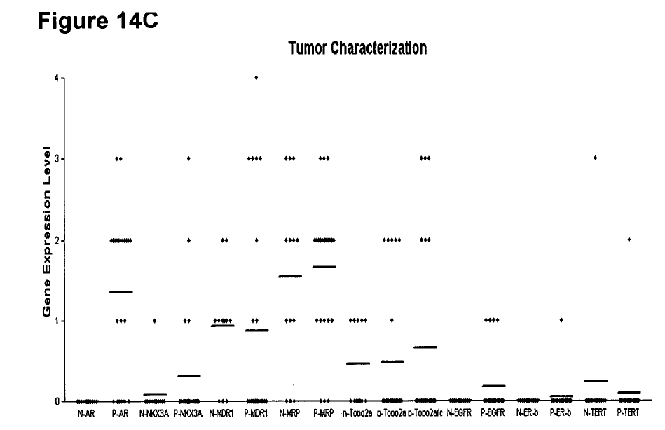

The gene survey goal was to identify mRNA expression profiles in CTC with the highest clinical specificity and sensitivity for detecting epithelial cells. First, gene expression levels found in the WBC was evaluated from enrichment of healthy donors. The selection of gene candidates was based on known literature data that showed broad epithelial specific expression levels. Identification of candidates that were negative in WBC would enable CTC profiling for categorizations/characterization on three basic histological levels, epithelial origin (FIG. 14A), tumor/organ of origin (FIG. 14B), and tumor therapeutic target characterization (FIG. 14C).

RT-PCR profiling was conducted on all samples according to the amplicon sets in FIGS. 14A, 14B, and 14C. FIG. 14A shows epithelial marker CK19 is 100% specific in the system of the present invention for HRPC samples, where 0/13 healthy donors were CK19 positive compared to 18/23 (78%). HRPC patient samples scored CK19 positive. CD5 and TROP2 are also 100% specific. However, their poor sensitivity of 1/23 (4%) and 2/23 (9%) respectively may not add sufficient profile value to justify their use. Of the genes that are not 100% specific (EpCAM, Muc-1, and MIC-1), a WBC background threshold can be applied between levels 1 and 2, above which all patient derived signals greater than one can be considered true positives. When profiling for maximum epithelial sensitivity with this set of genes, the combination of CK19, EpCAM, and Muc-1 sensitivity, 23/23 (100%) patient samples scored epithelial-positive. Since all blood samples yield a carry-over of WBC due to a low non-specific binding from the ferrofluid enrichment process, a WBC specific gene (F6=alpha 1-globin gene) was employed as the overall system positive control for RNA processing and amplification. All 36 samples scored greater than level 4 (see FIG. 14A).

### EXAMPLE 11

Assessing the Reproducibility in the Lower Limit Sensitivity of Preamplification

To evaluate the reproducible lower limit sensitivity performance of the modified T7 transcript preprocessing amplification procedure, a model system was constructed incorporating all the manipulations performed on the clinical RNA samples. Total RNA from breast and prostate cancer cell lines SKBR-3 and LNCaP (America Type Culture Collection, Manassas, Va.) were isolated using Trizol reagent (Life Technologies Inc.) according to manufacturer's instructions. Before first strand synthesis serial dilutions were made corresponding to 2, 0.2, 0.02 SKBR-3 cell equivalents, each dilution was spiked into 2 ng of total RNA from Percoll-derived WBC. RT-PCR external CK19 standard curve determination was run in parallel with SKBR-3 dilution curve, resulting in about 1,000 wild type CK19 mRNA copies present per SKBR-3 cell equivalent. The CK19 characterized SKBR-3 dilution curve was used as one of two types of specific transcripts to model the lower limit sensitivity and reproducibility of the total process of this T7-based mRNA library amplification system. The second transcript was an exogenous Lambda DNA based construct (Walker Biotech Publication) producing a 1.2 kb polyA(30) transcript. The curve for WBC's spiked with SKBR-3 cells was assayed in triplicate at each of the 2000, 200 and 20 CK19 mRNA copy levels. Lambda curves were spiked in triplicate into 2 ng total RNA from Percoll-derived WBC at the 500, 50, and 5 copy levels. In the final analysis, reproducible RT-PCR amplification was achieved from the 50 copy and greater levels (N=12) where individual samples were run through the entire T7-transcript preamplification each of which resulted in measurable signals. Furthermore, this lower limit of detection, starting with only 50 mRNA transcripts of any one sequence, was reproduced in a subsequent cell line spike model where all six sequence types (PSA, PSM, AR, HPN CK19 and EpCam) were serially diluted to known copy levels prior to aRNA and quantitative RT-PCR analysis (Example 6).

After RT-PCR, molecular weight and spot densitometry gel analysis was performed with an Alphalmager on aliquots from the 31, 35, and 40 cycle kinetics curves. A CV of 19.25% was calculated from 11 of the 12 spike levels (92%). Only one of the 200 copy samples (8%) had an intensity of 14 fold less than expected, but this sample was still qualitatively detectable, and was assigned as a level 1.

In a separate study, the effect of the T7 preamplification method of the present invention on relative mRNA abundance was modeled by comparison to identically prepared non-amplified libraries. No significant differences in band intensity ratios for N=8 genes (PSA, PSM, MGB1, MGB2, PIP, CK8, CK19, and EpCAM) were detected when starting with 15 cell equivalents of prostate cancer cell line LNCaP plus 15 cell equivalents of breast cancer cell line. SKBR-3 spiked into 1000 cell equivalents of WBC total RNA (2 ng) followed by the T7 preamplification method and subsequent multigene RT-PCR kinetic curve analyses, as shown in FIG. 13C. cDNA template negative amplification controls run in parallel with each batch showed no detectable signals during these studies.

Total RNA libraries were proportionately amplified using one round of the modified T7 method of the present invention yielding aRNA libraries with an average increase above the original mRNA mass of 10,000 fold. This is based on the original mRNA level estimation of 1.5% of the determined total RNA mass. The transcript amplification process resulted in libraries with a medium transcript length of 600 bases, which range between 550-800 bases. Individual transcript sizes within each library ranged from 300-3000 bases. Individual aRNA libraries were randomly primed for RT, from which a multigene panel of individual PCR reactions was performed using 10 donor equivalents of aRNA/cDNA.

Total RNA quantities from carried-over WBC's in healthy non-cancer samples ranged from 0.8-11.12 ng (mean 3.5 ng). Total RNA quantities from HRPC patient samples ranged from 0.8-35.12 ng (mean 7.2 ng). All total RNA samples subsequently produced aRNA libraries of masses directly proportional to the starting total RNA values.

A Northern blot of 10% of each sample's total RNA was hybridized with 28S plus 18S radiolabeled oligo probes in parallel with a known mass of total RNA from cell line standards which was followed by phosphoimage for quantity and quality determinations. Quality was assessed by ratio of 28S over 18S quantities where SKBR-3 cell line standard was averaged 1.55 (range 1.50-1.64), for the enriched samples, 13 healthy donors averaged 1.36 (range 1.16-1.60), HRPC averaged of 1.10 (ranged 0.57-1.80).

In addition, we observed that 80% of ferrofluid enriched CTC/WBC samples from HRPC patients had 6× less total RNA mass than expected (ranging 1.5 to 15 fold). These expectations were based on the WBC average total RNA mass=2 pg per cell and the average epithelial cell total RNA mass=20 pg per cell. This may be meaningful in assessing diagnostic and therapeutic status of individuals, especially during the course of treatments.

### EXAMPLE 12

Verification of CTC Tumor Tissue of Origin and Patient Specific Therapeutic Profile Charactization Using Tissue Specific Genes

The 36 samples were further profiled with N=8 amplicons to determine the optimum specificity and sensitivity expression profile for identification of a circulating epithelial tumor cell's organ of origin. For prostate specific identification we evaluated PSA, PSM, HK2, HPN, PSGR, DD3, MGB1 and MGB2 (see FIG. 14B). None of these amplicons showed any signal from the WBC group except for one outlier female #3 with Hepsin (HPN). As shown in FIG. 14B, PSA is the most sensitive for this prostate group with 20/23 (89%) samples scoring positive, followed by PSM 17/23 (74%), HPM 13/23 (57%), hK2 7/23 (30%), PSGR 2/23 (9%), and DD3 1/23 (4%). Unexpectedly, both “breast specific” genes, mamaglobin 1 and 2, scored strong signal level positives MBG1 1/23 (4%) and MBG2 2/23 (9%) in the prostate, but not in the healthy donor population. Combining the two and highest sensitivity markers PSA and PSM yields a sensitivity of 20/23 (87%). With the addition of HPN, the sensitivity increases to 21/23 (91%). Hepsin and PSM are two markers, which are also central to therapeutic strategies since they are both tissue specific and transmembrane enabling specific cell targeted delivery strategies.

Individual patient characterizations of potential therapeutic profile base on CTC RNA profiling was conducted and the results are show in FIG. 14C. Here individual specificity scores in this sample set showed AR, NKX3A, EGFR, and ER were all 100%. MDR1, MRP, and Topo2a all suffered from significant background signals from the WBC group. The sensitivity of AR for detecting HRPC CTC was 16/23 (70%) followed by NKX3A 4/23 (17%), Topo2a 5/23 (22%), MDR15/23 (22%), EGFR 4/23 (17%), and ER 1/23 (4%). Unexpectedly, MDR and MRP did not appear useful in stratifying these two groups.

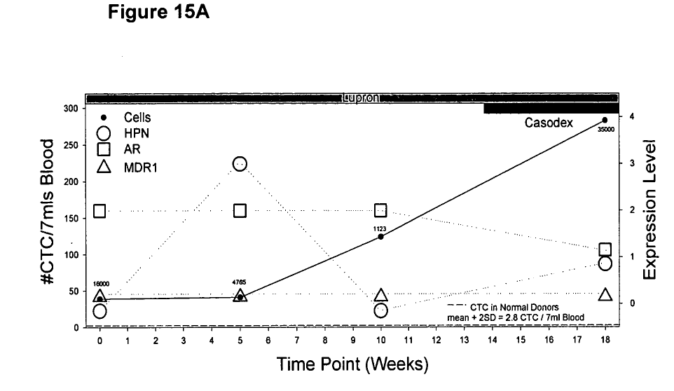

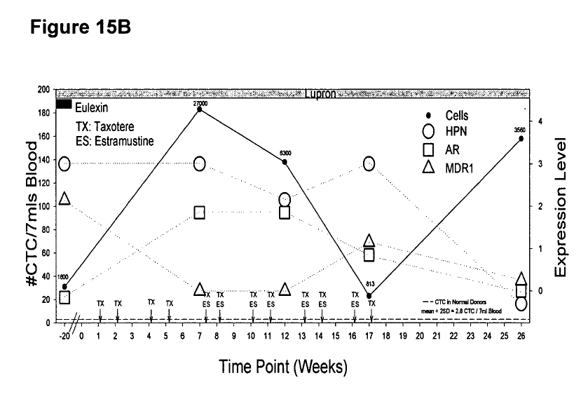

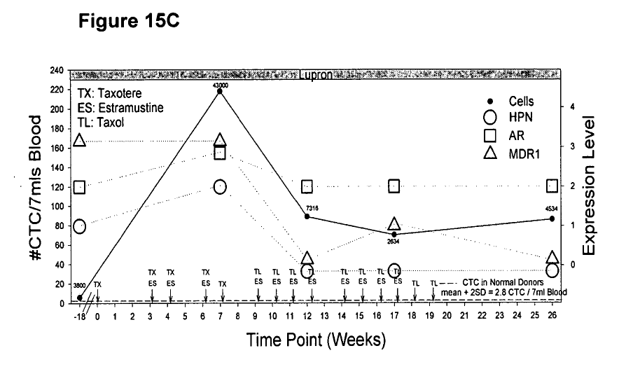

Longitudinal samples were drawn over the course of 18-26 weeks from three patients. One patient was treated with Lupron alone and two with Lupron combined with Taxane/Estramustine. Serial samples showed changes in the expression of therapeutic sensitivity/resistance associated genes, whereas others remained unchanged. These changes were independent of CTC and leukocyte counts, as shown in FIGS. 15A, 15B, and 15C. For the patient treated with Lupron alone, which had 4 longitudinal samples drawn at 0, 5, 10, and 18 weeks, MDR1 was not detectable (FIG. 15A), and AR remained relatively constant while Hespin fluctuated. FIGS. 15B and 15C further showed a consistent reduction of MDR1 mRNA levels relative to healthy donors during the course of TX/ES treatment. AR expression levels, which play a central role in the development of HRPC, were completely eliminated during TX/ES treatment as shown in FIG. 15B. In contrast, FIG. 15C showed AR relatively unaffected during a similar course of treatment. A dramatic change was detected in Hepsin mRNA from high expression levels for untreated to complete elimination during the TX/ES treatment of both patients in FIGS. 15B and 15C.

### EXAMPLE 13

Profiling Disease Status with Plasma-Derived (Non-CTC) RNA from The Same Sample Providing CTC-Complimentary Data

Heretofore, methods have been described for analyzing mRNA derived from CTC that are enriched from blood samples. An important step of this method is the T7 pre-amplification step, which allows the analysis of just a few copies of the transcript wit up to 1000 different individual gene specific RT-PCR reactions. T7 pre-amplification of representative mRNA libraries effectively removes the major restriction of limited sample mRNA mass. This same pre-amplification can be applied to non-CTC RNA. Indeed, there are numerous sources of RNA in a given blood sample, and some of these non-CTC RNA transcript will provide valuable information.

Confirmation of CTC presence and determination of tumor tissue of origin, as well as comprehensive characterization of disease mechanisms can be achieved using RNA derived from the plasma blood fraction obtained during the ferrofluid enrichment process. Preferably, this would be coupled with the T7 pre-amplification process described in the above examples for enriched CTC. The ferrofluid enrichment process initially separates out the blood plasma fraction of each sample. Typically, this fraction has been discarded as the CTC are enriched.

However, plasma- and serum-derived mRNA and DNA have recently been shown in the literature to provide valuable cancer expression (phenotype) and genotype (DNA analysis) data. Plasma-derived mRNA and DNA are isolated by traditional molecular biology methods for downstream analysis. Since mRNA is readily available from plasma, and has been demonstrated to provide valuable RT-PCR data, these same RNA transcripts can be more comprehensively profiled using the modified T7 amplification procedure describe herein. Thus, CTC-independent and/or CTC-complimentary mRNA expression profiles can be generated with the same profiling procedures for CTC by using the RNA from the plasma-derived fraction of each sample.

Furthermore, the T7 based expression profiling approach can be applied to the above described enrichment process, allowing analysis of the CTC-depleted fraction can be useful for differentiating the contributions of the WBC expression profile, which is non-specifically carried over during enrichment, and the contributions form the CTC-specific profile. This can be accomplished by differential pattern comparison and subsequent subtractions, providing an additional mechanism for correctly identifying CTC during analysis. In addition, the CTC-depleted profiles themselves will provide valuable patient-specific information regarding response and sensitivities to particular therapies.

It will be appreciated to those skilled in the art to which this invention relates that the invention is not limited to the descriptions and discussion of preferred embodiments disclosed herein, but that many modifications and variations of the procedures specifically described herein can be accomplished without departing from the spirit and scope of the invention, which is defined solely by the following claims.

What is claimed is:

1. A method for extracting intact cytoplasmic biomolecules from cells, which method comprises:
a. obtaining a fluid sample from a test subject, said fluid sample comprising a mixture of cell populations suspected of containing target cells;
b. releasing said cytoplasmic biomolecules from said cells;
c. isolating said cytoplasmic biomolecules; and
d. analyzing said cytoplasmic biomolecules.

2. The method as claimed in claim 1, wherein said fluid sample is whole blood.

3. The method as claimed in claim 1, wherein selection of said target cells is accomplished by a selecting method from the group consisting of immunomagnetic selection, cell fractionation, and combinations thereof.

4. The method according to claim 1, wherein said releasing step is accomplished by addition of a permeabilizing agent.

5. The method according to claim 4, wherein said permeabilizing agent is selected from the group consisting of a detergent, surfactant, and combinations thereof.

6. The method according to claim 4, wherein said permeabilizing agent is selected from the group consisting of saponin, Immuniperm, and combinations thereof.

7. The method according to claim 4, wherein said permeabilizing agent is Immuniperm.

8. The method according to claim 1, wherein said obtaining said fluid sample includes treating the sample with a cell stabilizing agent.

9. The method according to claim 8, wherein said stabilizing agent is selected from the group consisting of aldehydes, urea, and combinations thereof.

10. The method according to claim 9, wherein said aldehyde is selected from the group consisting of formaldehyde, paraformaldehyde, and combinations thereof.

11. The method according to claim 8, wherein said stabilizing agent is a dialdehyde.

12. The method according to claim 11, wherein said dialdehyde is selected from the group consisting of glutaraldehyde, glyoxal, and combinations thereof.

13. The method according to claim 8, wherein said releasing of said cytoplasmic biomolecules involves macromolecular complexes formed after exposure to said stabilizing agent.

14. The method according to claim 13, wherein said releasing said cytoplasmic biomolecules from said cells is accomplished with enzymatic digestion.

15. The method according to claim 14, wherein said enzymatic digestion is accomplished with a selection from the group of proteinases, nucleophiles, and combinations thereof.

16. The method according to claim 15, wherein said proteinases are selected from the group consisting of proteinase K digestion, V8 proteinase digestion, pronase digestion, and combinations thereof.

17. The method according to claim 15, wherein said nucleophiles are from a group consisting of phosphate-based buffers, tris-based buffers, acetic hydrazide, hydroxylamine, and combinations thereof.

18. The method according to claim 1, wherein said cytoplasmic biomolecules are proteins.

19. The method according to claim 18, wherein said isolating said proteins is accomplished by a method selected from the group consisting of chemical extractions, electrophoresis, chromatography, immunoseparations and affinity techniques.

20. The method according to claim 1, wherein said cytoplasmic biomolecules are nucleic acids.

21. The method according to claim 20, wherein said nucleic acids are selected from the group consisting of cytoplasmic RNA, nuclear and mitochondrial RNA, nuclear and mitochondrial DNA, and combinations thereof.

22. The method according to claim 20, wherein said nucleic acids are cytoplasmic mRNA.

23. The method according to claim 20, wherein the method of isolating said nucleic acids is selected from the group consisting of RNA or DNA chemical extractions, electrophoresis, chromatography, immunoseparations and affinity techniques.

24. The method according to claim 20, wherein said isolating of said nucleic acids is accomplished by capture with magnetic beads affixed to oligo(dT).

25. The method according to claim 1, wherein said target cells are assessed for phenotypic expression other than with said cytoplasmic biomolecules after obtaining said fluid sample.

26. The method according to claim 25, wherein said phenotypic expression is selected from the group consisting of morphological examining, cell component staining, immunoanalyzing, and combinations thereof.

27. The method according to claim 1, wherein said analysis of said cytoplasmic biomolecules is accomplished by functional proteomics.

28. The method according to claim 27, wherein said functional proteomics is selected from the group consisting of protein expression profile, Western blot, amino acid sequence analysis, electrophoresis, 2-D electrophoresis, mass spectrometry, gas chromatography, liquid chromatography nuclear magnetic resonance, infrared, atomic adsorption, and combinations thereof.

29. The method according to claim 1, wherein said analyzing of said cytoplasmic biomolecules is accomplished by functional genomics.

30. The method according to claim 29, wherein said functional genomics is selected from the group consisting of mRNA profile analysis, protein expression profile analysis, polymerase chain reaction, Northern blot, Western blot, Nucleotide or amino acid sequence analysis, gene expression on a microarray, electrophoresis, 2-D electrophoresis, mass spectrometry, gas chromatography, liquid chromatography nuclear magnetic resonance, infrared, and atomic adsorption.

31. The method according to claim 1, wherein said analyzing of said cytoplasmic RNA for the presence of at least two genetic markers is accomplished by multigene RT-PCR.

32. The method according to claim 1, wherein said analyzing of said cytoplasmic RNA for said genetic markers comprises the further steps of:
a. reverse transcripting said genetic markers with at least one set of gene specific primers all of which have a universal primer extension on the 5′ terminus;
b. removing said gene specific primers;
c. amplifying a gene specific amplicon with said universal primer extensions in a PCR amplification; and
d. identifying said gene specific amplicon.

33. The method according to claim 32, wherein said removing of said gene specific primers is accomplished using a method selected from the group consisting of molecular size exclusion, solid support selective attachment, and single strand specific DNase, and thereafter incorporating uracil-N-glycosylase with DNA oligonucleotide primers synthesized with deoxyUridine.

34. The method according to claim 32, wherein said removing of said gene specific primers is accomplished with uracil-N-glycosylase treatment of DNA oligonucleotide primers synthesized with deoxyUridine.

35. The method according to claim 34, wherein said uracil-N-glycosylase treatment is followed by incorporation of DNase-free RNases.

36. The method according to claim 33, wherein said gene specific primers are used under conditions of high primer-target annealing specificity.

37. The method according to claim 36, wherein said high primer-target annealing specificity is accomplished with proteins from natural recombination cellular repair mechanisms.

38. The method according to claim 37, wherein said high primer-target annealing specificity is accomplished with recA.

39. The method according to claim 32, wherein said identifying said gene specific amplicon is accomplished by its unique Rf value in size-based analysis systems.

40. The method according to claim 39, wherein said size-based analysis systems are selected from the group consisting of PAGE, agarose gel electrophoresis, capillary gel electrophoresis, SELDI, MALDI, and cDNA arrays.

41. The method according to claim 1, wherein said analyzing step further comprises:
a. preamplifying said nucleic acids extracted by linear amplification wherein said preamplifying results in at least 1000 fold increase in all library transcripts in the from of aRNA;
b. synthesizing a second strand from said aRNA only for up to 1000 independent selected genes of interest; and
c. recognizing an amplified product using a method selected from the group consisting of array analysis, electrophoresis, and combinations thereof.

42. The method according to claim 41, wherein said preamplifying is accomplished by using a polymerase promoter selected from the group consisting of SP6 RNA polymerase promoter, T3 RNA polymerase promoter, and T7 RNA polymerase promoter.

43. The method according to claim 41, wherein said preamplifing is accomplished by using RNA polymerase using random primers.

44. The method according to claim 41, wherein said synthesizing of said second strand is under conditions of high primer-target annealing specificity.

45. The method according to claim 41, wherein said high primer-target annealing specificity is accomplished with proteins from natural recombination cellular repair mechanisms.

46. The method according to claim 41, wherein said high primer-target annealing specificity is accomplished with recA.

47. The method according to claim 41, wherein said recognizing is by pretreatment with DNase-free RNases.

48. The method according to claim 47, wherein said pretreatment is accomplished by performing a method selected from the group consisting of phenol extraction, silica binding, and combinations thereof.

49. The method according to claim 41, wherein said recognizing is accomplished by amplification of all double-stranded products.

50. The method according to claim 41, wherein said recognizing is accomplished by performing a method selected from the group consisting of array analysis, electrophoresis, and combinations thereof.

51. A method for improving signal-to-noise in the detection of distinct target sequences, which method comprises:
a. isolating nucleic acids;
b. preamplifying said nucleic acids by linear amplification wherein said preamplifying results in at least 1000 fold increase in all library transcripts in the from of aRNA;
c. synthesizing a second strand from said aRNA only for at least 2 independent selected genes of interest;
d. purifying double-stranded product; and
e. identifying said product.

52. The method according to claim 51, wherein said nucleic acids are selected from the group consisting of cytoplasmic RNA, nuclear and mitochondrial RNA, nuclear and mitochondrial DNA, and combinations thereof.

53. The method according to claim 51, wherein said nucleic acids are cytoplasmic mRNA.

54. The method according to claim 51, wherein said isolating of said nucleic acids is accomplished by performing a method selected from the group consisting of RNA or DNA chemical extractions, electrophoresis, chromatography, immunoseparations and affinity techniques.

55. The method according to claim 51, wherein said isolating of said nucleic acids is by capture with magnetic beads affixed to oligo(dT).

56. The method according to claim 51, wherein said preamplifying is by using a polymerase/promoter selected from the group consisting of SP6 RNA polymerase/promoter, T3 RNA polymerase/promoter, and T7 RNA polymerase/promoter.

57. The method according to claim 51, wherein said preamplifing is accomplished with RNA polymerase using random primers.

58. The method according to claim 51, wherein said synthesizing of said second strand is under conditions of high primer-target annealing specificity.

59. The method according to claim 58, wherein said high primer-target annealing specificity is accomplished with proteins from natural recombination cellular repair mechanisms.

60. The method according to claim 58, wherein said high primer-target annealing specificity is accomplished with recA.

61. The method according to claim 51, wherein said purifying said double-stranded products is accomplished with DNase-free RNases.

62. The method according to claim 51, wherein said purifying is accomplished by treatment from a group consisting of phenol extraction, silica binding, and combinations thereof.

63. The method according to claim 51, wherein said identifying is accomplished by amplification of said double stranded products.

64. The method according to claim 51, wherein said identifying step is accomplished by a method selected from the group consisting of array analysis, electrophoresis, and combinations thereof.

65. A method for acquiring intact cytoplasmic biomolecules from cells, which method comprises:
a. obtaining a fluid sample from a test subject, said fluid sample comprising a mixture of cell populations suspected of containing target cytoplasmic biomolecules;
b. fixing said cell populations;
c. recovering said cytoplasmic biomolecules; and
d. analyzing said cytoplasmic biomolecules.

66. The method according to claim 65, wherein said fixing is accomplished by treatment with a cell stabilizing agent.

67. The method according to claim 66, wherein said stabilizing agent is selected from the group consisting of aldehydes, urea, and combinations thereof.

68. The method according to claim 67, wherein said aldehyde is selected from the group consisting of formaldehyde, paraformaldehyde, and combinations thereof.

69. The method according to claim 68, wherein said stabilizing agent is a dialdehyde.

70. The method according to claim 69, wherein said dialdehyde is selected from the group consisting of glutaraldehyde, glyoxal, and combinations thereof.

71. The method according to claim 65, wherein said recovering of said cytoplasmic biomolecules is accomplished from macromolecular complexes formed after exposure to said stabilizing agent.

72. The method according to claim 71, wherein said recovering said cytoplasmic biomolecules from said cells is accomplished by enzymatic digestion.

73. The method according to claim 72, wherein said enzymatic digestion is accomplished by using a member selected from the group consisting of proteinases, nucleophiles, and combinations thereof.

74. The method according to claim 73, wherein said nucleophiles are selected from the group consisting of phosphate-based buffers, tris-based buffers, acetic hydrazide, hydroxylamine, and combinations thereof.

75. The method according to claim 73, wherein said proteinases are selected from the group consisting of proteinase K digestion, V8 proteinase digestion, pronase digestion, and combinations thereof.

76. The method according to claim 65, wherein said cytoplasmic biomolecules are proteins.

77. The method according to claim 76, wherein said recovering of said proteins is accomplished by a method selected from the group consisting of chemical extractions, electrophoresis, chromatography, immunoseparations and affinity techniques.

78. The method according to claim 65, wherein said cytoplasmic biomolecules are nucleic acids.

79. The method according to claim 78, wherein said nucleic acids are selected from the group consisting of cytoplasmic RNA, nuclear and mitochondrial RNA, nuclear and mitochondrial DNA, and combinations thereof.

80. The method according to claim 78, wherein said nucleic acids are cytoplasmic mRNA.

81. The method according to claim 78, wherein said analyzing of said nucleic acids is accomplished by a method selected from the group consisting of RNA or DNA chemical extractions, electrophoresis, chromatography, immunoseparations and affinity techniques.

82. The method according to claim 78, wherein said analyzing of said nucleic acids is accomplished by capture with magnetic beads affixed to oligo(dT).

83. A method for amplifying multiple genetic markers in the same reaction mix, which method comprises:
a. reverse transcripting said genetic markers with at least one set of gene specific primers all of which have a universal primer extension on the 5′ terminus;
b. removing said gene specific primers;
c. amplifying a gene specific amplicon with said universal primer extensions in a PCR amplification; and
d. identifying said gene specific amplicon.

84. The method according to claim 83, wherein said removing of said gene specific primers is accomplished by a method selected from the group consisting of molecular size exclusion, solid support selective attachment, single strand specific DNase, and incorporating uracil-N-glycosylase with DNA oligonucleotide primers synthesized with deoxyuridine.

85. The method according to claim 83, wherein said removing of said gene specific primers is accomplished with uracil-N-glycosylase treatment of DNA oligonucleotide primers synthesized with deoxyuridine.

86. The method according to claim 85, wherein said uracil-N-glycosylase treatment is followed by incorporation of DNase-free RNases.

87. The method according to claim 83, wherein said gene specific primers are used under conditions of high primer-target annealing specificity.

88. The method according to claim 87, wherein said high primer-target annealing specificity is accomplished with proteins from natural recombination cellular repair mechanisms.

89. The method according to claim 88, wherein said high primer-target annealing specificity is accomplished with recA.

90. The method according to claim 83, wherein said identifying said gene specific amplicon is accomplished by its unique Rf value in size-based analysis systems.

91. The method according to claim 90, wherein said size-based analysis systems are selected from the group consisting of PAGE, agarose gel electrophoresis, capillary gel electrophoresis, SELDI, MALDI, and cDNA arrays.

92. Apparatus for extracting intact cytoplasmic biomolecules from cells, comprising:
a. means for obtaining a fluid sample from a test subject, said fluid sample comprising a mixture of cell populations suspected of containing target cells;
a. means for releasing said cytoplasmic biomolecules from said cells;
b. means for isolating said cytoplasmic biomolecules; and
c. means for analyzing said cytoplasmic biomolecules.

93. Apparatus as claimed in claim 92, wherein said fluid sample is whole blood.

94. Apparatus as claimed in claim 92, wherein said means for obtaining said fluid sample is by a group consisting of immunomagnetic selection means, cell fractionation means, and combination means thereof.

95. Apparatus according to claim 92, wherein said means for releasing is by permeabilizing agent addition.

96. Apparatus according to claim 97, wherein said permeabilizing agent is from a group consisting of a detergent, surfactant, and combinations thereof.

97. Apparatus according to claim 95, wherein said permeabilizing agent is from a group consisting of saponin, Immuniperm, and combinations thereof.

98. Apparatus according to claim 95, wherein said permeabilizing agent is Immuniperm.

99. Apparatus according to claim 92, wherein means for obtaining said fluid sample is by means for treatment with cell stabilizing agent.

100. Apparatus according to claim 99, wherein said stabilizing agent is from a group consisting of aldehydes, urea, and combinations thereof.

101. Apparatus according to claim 100, wherein said aldehyde is from a group consisting of formaldehyde, paraformaldehyde, and combinations thereof.

102. Apparatus according to claim 100, wherein said stabilizing agent is a dialdehyde.

103. Apparatus according to claim 102, wherein said dialdehyde is from a group consisting of glutaraldehyde, glyoxal, and combinations thereof.

104. Apparatus according to claim 99, wherein said means for releasing of said cytoplasmic biomolecules are from macromolecular complexes formed after exposure to said stabilizing agent.

105. Apparatus according to claim 99, wherein said means for releasing said cytoplasmic biomolecules from said cells through enzymatic digestion means.

106. Apparatus according to claim 105, wherein said means for enzymatic digestion occurs through a group consisting of proteinase means, nucleophile means, and combination means thereof.

107. Apparatus according to claim 106, wherein said nucleophile means is from a group consisting of phosphate-based buffer means, tris-based buffer means, acetic hydrazide means, hydroxylamine means, and combination means thereof.

108. Apparatus according to claim 106, wherein said proteinase means is from a group consisting of proteinase K digestion means, V8 proteinase digestion means, pronase digestion means, and combination means thereof.

109. Apparatus according to claim 92, wherein said cytoplasmic biomolecules are proteins.

110. Apparatus according to claim 109, wherein said means for isolating said proteins is from a group consisting of chemical extractions means, electrophoresis means, chromatography means, immunoseparation means and affinity techniques means.

111. Apparatus according to claim 92, wherein said cytoplasmic biomolecules are nucleic acids.

112. Apparatus according to claim 114, wherein said nucleic acids are from a group consisting of cytoplasmic RNA, nuclear and mitochondrial RNA, nuclear and mitochondrial DNA, and combinations thereof.

113. Apparatus according to claim 111, wherein said nucleic acids are cytoplasmic mRNA.

114. Apparatus according to claim 111, wherein said means for isolating of said nucleic acids is from a group consisting of RNA or DNA chemical extractions means, electrophoresis means, chromatography means, immunoseparation means and affinity techniques means.

115. Apparatus according to claim 113, wherein said means for said isolating is by capturing means with magnetic beads affixed to oligo(dT).

116. Apparatus according to claim 92, wherein means for assessing target cell phenotypic expression other than with said cytoplasmic biomolecules.

117. Apparatus according to claim 116, wherein said means of phenotypic expression is from a group consisting of morphological examinating means, cell component staining means, immunoanalyzing means, and combination means thereof.

118. Apparatus according to claim 92, wherein said means for analyzing said cytoplasmic biomolecules is by functional proteomics means.

119. Apparatus according to claim 118, wherein said means for functional proteomics is from a group consisting of protein expression profile means, Western blot means, amino acid sequence analysis means, electrophoresis means, 2-D electrophoresis means, mass spectrometry means, gas chromatography means, liquid chromatography means, nuclear magnetic resonance means, infrared means, atomic adsorption means, and combination means thereof.

120. Apparatus according to claim 92, wherein said means for analyzing said cytoplasmic biomolecules is by functional genomics means.

121. Apparatus according to claim 120, wherein said functional genomics means is from a group consisting of mRNA profile analysis means, protein expression profile analysis means, polymerase chain reaction means, Northern blot means, Western blot means, Nucleotide or amino acid sequence analysis means, means of gene expression on a microarray, electrophoresis means, 2-D electrophoresis means, mass spectrometry means, gas chromatography means, liquid chromatography means, nuclear magnetic resonance means, infrared means, and atomic adsorption means.

122. Apparatus according to claim 92, wherein said means for analyzing said cytoplasmic RNA for the presence of at least two genetic markers is by multigene RT-PCR means.

123. Apparatus according to claim 92, wherein said means for analyzing said cytoplasmic RNA for said genetic markers comprises:
a. means for reverse transcripting said genetic markers with at least one set of gene specific primers all of which have a universal primer extension on the 5′ terminus;
b. means for removing said gene specific primers;
c. means for amplifying a gene specific amplicon with said universal primer extensions in a PCR amplification; and
d. means for identifying said gene specific amplicon.

124. Apparatus according to claim 123, wherein said means for removing of said gene specific primers is from a group consisting of molecular size exclusion means, solid support selective attachment means, single strand specific DNase means, and means for incorporating uracil-N-glycosylase with DNA oligonucleotide primers synthesized with deoxyuridine.

125. Apparatus according to claim 123, wherein said means for removing of said gene specific primers is by means for uracil-N-glycosylase treatment of DNA oligonucleotide primers synthesized with deoxyUridine.

126. Apparatus according to claim 123, wherein said means for removing said gene specific primers is by means for incorporation of DNase-free RNases.

127. Apparatus according to claim 123, wherein said gene specific primers are used with means for conditions of high primer-target annealing specificity.

128. Apparatus according to claim 127, wherein said means for high primer-target annealing specificity is with proteins from natural recombination cellular repair mechanisms.

129. Apparatus according to claim 128, wherein said means for high primer-target annealing specificity is with recA.

130. Apparatus according to claim 123, wherein said means for identifying said gene specific amplicon is by means using unique Rf value in size-based analysis systems.

131. Apparatus according to claim 130, wherein said means for size-based analysis systems is from a group consisting of PAGE means, agarose gel electrophoresis means, capillary gel electrophoresis means, SELDI means, MALDI means, and means for cDNA arrays.

132. Apparatus according to claim 92, wherein said analyzing means comprises:
a. means for preamplifying said nucleic acids extracted by linear amplification means wherein said preamplifying means results in at least 1000 fold increase in all library transcripts in the from of aRNA;
b. means for synthesizing a second strand from said aRNA only for up to 1000 independent selected genes of interest; and
c. means for recognizing an amplified product from a group consisting of array analysis means, electrophoresis means, and combination means thereof.

133. Apparatus according to claim 132, wherein said means for preamplifying is from a group consisting of SP6 RNA polymerase promoter means, T3 RNA polymerase promoter means, and T7 RNA polymerase promoter means.

134. Apparatus according to claim 132, wherein said means for preamplifing is with RNA polymerase means using random primers.

135. Apparatus for improving signal-to-noise in the detection of distinct target sequences comprising:
a. means for isolating nucleic acids;
b. means for preamplifying said nucleic acids by linear amplification means wherein said means for preamplifying results in at least 1000 fold increase in all library transcripts in the from of aRNA;
c. means for synthesizing a second strand from said aRNA only for at least 2 independent selected genes of interest;
d. means for purifying double-stranded product; and
e. means for identifying said product.

136. Apparatus according to claim 135, wherein said nucleic acids are from a group consisting of cytoplasmic RNA, nuclear and mitochondrial RNA, nuclear and mitochondrial DNA, and combinations thereof.

137. Apparatus according to claim 135, wherein said nucleic acids are cytoplasmic mRNA.

138. Apparatus according to claim 135, wherein said means for isolating of said nucleic acids is from a group consisting of RNA or DNA chemical extractions means, electrophoresis means, chromatography means, immunoseparation means and affinity techniques means.

139. Apparatus according to claim 135, wherein said means for isolating of said nucleic acids is by capturing means with magnetic beads affixed to oligo(dT).

140. Apparatus according to claim 135, wherein said means for preamplifying is from a group consisting of SP6 RNA polymerase/promoter means, T3 RNA polymerase/promoter means, and T7 RNA polymerase/promoter means.

141. Apparatus according to claim 135, wherein said means for preamplifing is by RNA polymerase means using random primers.

142. Apparatus according to claim 135, wherein said means for synthesizing of said second strand is by means for conditions of high primer-target annealing specificity.

143. Apparatus according to claim 142, wherein said means for high primer-target annealing specificity is with proteins from natural recombination cellular repair mechanisms.

144. Apparatus according to claim 143, wherein said means for high primer-target annealing specificity is with recA.

148. Apparatus according to claim 138, wherein said means for purifying of said double stranded products is with DNase-free Rnases means.

148. Apparatus according to claim 135, wherein said means for purifying is from a group consisting of phenol extraction means, silica binding means, and combination means thereof.

149. Apparatus according to claim 135, wherein said means for identifying is by amplification means for said double stranded products.

150. Apparatus according to claim 149, wherein said means for identifying is by said amplification means from a group consisting of array analysis means, electrophoresis means, and combination means thereof.

151. Apparatus for acquiring intact cytoplasmic biomolecules from cells, comprising:
a. means for obtaining a fluid sample from a test subject, said fluid sample comprising a mixture of cell populations suspected of containing target cytoplasmic biomolecules;
d. means for fixing said cell populations;
e. means for recovering said cytoplasmic biomolecules; and
f. means for analyzing said cytoplasmic biomolecules.

152. Apparatus according to claim 151, wherein said means for fixing is by treatment means with a cell stabilizing agent.

153. Apparatus according to claim 152, wherein said stabilizing agent is from a group consisting of aldehydes, urea, and combinations thereof.

154. Apparatus according to claim 153, wherein said aldehyde is from a group consisting of formaldehyde, paraformaldehyde, and combinations thereof.

155. Apparatus according to claim 152, wherein said stabilizing agent is a dialdehyde.

156. Apparatus according to claim 155, wherein said dialdehyde is from a group consisting of glutaraldehyde, glyoxal, and combinations thereof.

157. Apparatus according to claim 152, wherein said means for recovering of said cytoplasmic biomolecules are from macromolecular complexes formed after exposure to said stabilizing agent.

158. Apparatus according to claim 152, wherein said means for recovering said cytoplasmic biomolecules from said cells is by enzymatic digestion means.

160. Apparatus according to claim 158, wherein said enzymatic digestion means is through a group consisting of proteinase means, nucleophile means, and combination means thereof.

161. Apparatus according to claim 160, wherein said nucleophile means is from a group consisting of phosphate-based buffer means, tris-based buffer means, acetic hydrazide means, hydroxylamine means, and combination means thereof.

162. Apparatus according to claim 160, wherein said proteinase means is from a group consisting of proteinase K digestion, V8 proteinase digestion, pronase digestion, and combinations thereof.

163. Apparatus according to claim 151, wherein said cytoplasmic biomolecules are proteins.

164. Apparatus according to claim 151, wherein said means for recovering said proteins is from a group consisting of chemical extraction means, electrophoresis means, chromatography means, immunoseparation means and affinity techniques means.

165. Apparatus according to claim 151, wherein said cytoplasmic biomolecules are nucleic acids.

166. Apparatus according to claim 165, wherein said nucleic acids are from a group consisting of cytoplasmic RNA, nuclear and mitochondrial RNA, nuclear and mitochondrial DNA, and combinations thereof.

167. Apparatus according to claim 165, wherein said nucleic acids are cytoplasmic mRNA.

168. Apparatus according to claim 165, wherein said means for analyzing of said nucleic acids is from a group consisting of RNA or DNA chemical extraction means, electrophoresis means, chromatography means, immunoseparation means and affinity techniques means.

169. The method according to claim 165, wherein said means for analyzing of said nucleic acids are by capture means with magnetic beads affixed to oligo(dT).

170. A cell preparation kit for maintaining phenotypic and genotypic integrity of a single cell or population of cells comprising:
a. target cell permeabilizing agents;
b. target cell stabilizing agents;
c. nucleic acid releasing agents; and
d. a protocol for using the method of claim 66.

171. The kit as claimed in claim 170, wherein said kit further containing immunomagnetic particles having binding specificity for said target cells.

172. The kit as claimed in claim 170, wherein said permeabilizing agent is selected from the group consisting of saponin, detergents, surfactants and combinations thereof.

173. The kit as claimed in claim 170, wherein said permeabilizing agent is Immuniperm.

174. The kit as claimed in claim 170, wherein said stabilizing agent is selected from the group consisting of aldehydes, dialdehydes, urea and combinations thereof.

175. The kit as claimed in claim 170, wherein said releasing agent is selected from the group consisting of proteinases, nucleophiles, and combinations thereof.

176. The kit as claimed in claim 175, wherein said proteinase is selected from the group consisting of proteinase K, V8 proteinase, pronase, and combinations thereof.

177. The kit as claimed in claim 175, wherein said nucleophile is selected from the group consisting of phosphate-based buffers, tris-based buffers, acetic hydrazide, hydroxylamine, and combinations thereof.

178. A nucleotide amplification kit for increasing the signal-to-noise ratio in distinct target sequences comprising:
a. nucleic acid isolating agents;
b. preamplifying agents wherein said preamplifying agents generate an aRNA library with at least a 1000 fold increase in all transcripts;
c. second strand synthesizing agents wherein said synthesizing agents target at least 2 independent selected transcripts of interest;
d. purifying agents wherein said purifying agents select for double-stranded products;
e. nucleotide specific detecting agents wherein said detecting agents enable confirmation of double-stranded products; and
f. a protocol for using the method of claim 52.

179. The kit as claimed in claim 178, wherein said isolating agents are immunomagnetic particles having binding specificity for said nucleic acids.

180. The kit as claimed in claim 178, wherein said isolating agents are from a group consisting of RNA or DNA chemical extracting agents, electrophoresis agents, chromatography agents, immunoseparation agents and nucleotide affinity agents.

181. The kit as claimed in claim 178, wherein said preamplifying agents are from a group consisting of SP6 RNA polymerase/promoter, T3 RNA polymerase/promoter, and T7 RNA polymerase/promoter.

182. The kit as claimed in claim 178, wherein said preamplifying agent is RNA polymerase/random primers.

183. The kit as claimed in claim 178, wherein said second strand synthesizing agents are polymerase/primers with high primer-target annealing specificity.

184. The kit as claimed in claim 178, wherein said purifying agents are DNase-free Rnases.

185. The kit as claimed in claim 178, wherein said purifying agents are from a group consisting of phenol extraction agents and silica binding agents.

186. The kit as claimed in claim 178, wherein said nucleotide specific detecting agents is from a group consisting of double-stranded nucleotide amplification agents, array detection agents, and electrophoresis agents.
